(19) United States
(12) Patent Application Publication
(10) Pub. No.: US 2024/0216145 A1
(43) Pub. Date: Jul. 4, 2024

(54) IMPLANTS, INSTRUMENTS, SYSTEMS, AND METHODS OF USING

(71) Applicant: Paragon 28, Inc., Englewood, CO (US)

(72) Inventors: Albert DACOSTA, Lone Tree, CO (US); Sean GILL, Denver, CO (US); Benjamin MAJORS, Englewood, CO (US); Laura Zagrocki BRINKER, Lone Tree, CO (US); Kaitlin KARAS, Denver, CO (US)

(73) Assignee: Paragon 28, Inc., Englewood, CO (US)

(21) Appl. No.: 18/431,309

(22) Filed: Feb. 2, 2024

Related U.S. Application Data
(63) Continuation of application No. PCT/US22/74447, filed on Aug. 2, 2022.
(60) Provisional application No. 63/203,852, filed on Aug. 2, 2021.

(51) Int. Cl. A61F 2/42; A61F 2/46; A61F 2/30

(52) U.S. Cl. CPC A61F 2/4202; A61F 2/4606; A61F 2/4684; A61F 2002/30261; A61F 2002/30622; A61F 2002/30777; A61F 2002/30785; A61F 2002/3092

(57) ABSTRACT

Implants, devices, instruments, systems and methods for using the same for correcting bone deformities in the lower extremity are disclosed. Specifically, implants, devices, instruments and systems used for ankle fusion procedures are disclosed. The implants including an interior frame, an exterior frame surrounding the interior frame, and a lattice structure extending between at least a portion of the interior frame and the exterior frame.

### CROSS REFERENCE TO RELATED APPLICATIONS

This application is a continuation of PCT/US2022/074447 filed on Aug. 2, 2022 and entitled Implants, Instruments, Systems, and Methods of Using, which claims priority benefit under 35 U.S.C. § 119(e) to U.S. provisional application No. 63/203,852 filed on Aug. 2, 2021 and entitled Implants, Instruments, Systems, and Methods of Using, which are incorporated herein by reference in their entireties.

### FIELD OF THE INVENTION

The present invention relates generally to general surgery, podiatric, and orthopaedic implants and instruments used for inserting implants to correct bone deformities. More specifically, but not exclusively, the present invention relates to implants, instruments, systems and methods for using the implants, instruments and systems to correct bone deformities.

### BACKGROUND OF THE INVENTION

Ankle fusion procedures are commonly performed through the combined use of hardware including screw fixation, intramedullary nails, and plate-screw combinations. These procedures are performed to treat osteo-arthritis and Charcot most typically.

A subset of ankle fusion cases involve large bone defects of the hindfoot, whereby any number of factors result in a talar void that must either be filled prior to fusion or result in leg shortening by a tibio-calcaneal fusion. Pathologies that can lead to a large talar bone void include Charcot, a failed total ankle replacement (TAR) needing to be removed, and/or avascular necrosis of the talus.

Currently the standard of care for filling these voids and associated shortcomings include femoral head allograft, custom patient specific instrumentation (PSI), or talocalcaneal (TC) fusion. Using a femoral head allograft procedure may result in possible resorption and structural weakness. Custom PSI may result in annual limits on number of procedures, higher demand for pre-operative planning. While TC fusion may result in leg shortening.

Further, for all currently available options it can be difficult to minimize healthy bone resection during preparation due to the highly variable and unpredictable patterns of bone loss associated with these cases. Even with PSI achieving a proper fit can be very challenging due to the gap between the anticipated void shape and/or size from pre-operative planning and the clinical reality intra-operatively.

Thus, new implants, instruments, systems, and procedures are needed for improved insertion of implants to correct bone deformities.

### SUMMARY OF THE INVENTION

Aspects of the present invention provide implants, instruments, systems, and methods for correcting bone deformities in the foot and/or ankle.

In one aspect, provided herein is an implant. The implant including, an interior frame, an external frame surrounding the interior frame, and a lattice structure extending between at least a portion of the interior frame and the external frame.

In another aspect, provided herein is a system. The system including, an implant and an insertion instrument coupled to the external frame of the implant. The implant including, an interior frame, an external frame surrounding the interior frame, and a lattice structure extending between at least a portion of the interior frame and the external frame.

In yet another aspect, provided herein is an insertion instrument. The insertion instrument including, a handle, an external shaft coupled to the handle on a first end, and an internal shaft rotatably received within the external shaft.

In still another aspect, provided herein is a precision guide. The precision guide including, an arm and a guide tube received within the arm. The arm including a first portion, a second portion, and a coupling portion coupled to the first portion on a first end and the second portion on a second end, wherein the coupling portion angles the first portion relative to the second portion.

In another aspect, provided herein is a precision guide. The precision guide including, a frame, a guide tube received within the frame, and at least one angle wing received within the frame.

In a further aspect, provided herein is a surgical method. The surgical method includes, exposing a patient's joint and creating a cavity in the joint to receive an implant. The method also includes inserting trial implants into the cavity to select a desired implant size and selecting the implant corresponding to the trial implant with the desire implant size. The method further includes coupling the implant to an insertion instrument and inserting the implant coupled to the insertion instrument into the cavity. In addition, the method includes manipulating the coupled implant and insertion instrument in at least one of rotation and translation to a desired position and fixing the implant to the joint.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a first perspective view of an implant, in accordance with an aspect of the present disclosure;

FIG. 2 is a second perspective view of the implant of FIG. 1, in accordance with an aspect of the present disclosure;

FIG. 3 is a first end view of the implant of FIG. 1, in accordance with an aspect of the present disclosure;

FIG. 4 is a second end view of the implant of FIG. 1, in accordance with an aspect of the present disclosure;

FIG. 5 is a first side view of the implant of FIG. 1, in accordance with an aspect of the present disclosure;

FIG. 6 is a second side view of the implant of FIG. 1, in accordance with an aspect of the present disclosure;

FIG. 7 is a top view of the implant of FIG. 1, in accordance with an aspect of the present disclosure;

FIG. 8 is a bottom view of the implant of FIG. 1, in accordance with an aspect of the present disclosure;

FIG. 9 is a cross-sectional view of the implant of FIG. 1 taken along line 9-9 in FIG. 5, in accordance with an aspect of the present disclosure;

FIG. 10 is a cross-sectional view of the implant of FIG. 1 taken along line 10-10 in FIG. 3, in accordance with an aspect of the present disclosure;

FIG. 11 is another perspective view of the implant of FIG. 1 including a lattice structure inserted into the openings of the implant of FIG. 1, in accordance with an aspect of the present disclosure;

FIG. 12 is a top view of a portion of the lattice structure inserted into the implant of FIG. 11, in accordance with an aspect of the present disclosure;

FIG. 13 is a first perspective view of another implant, in accordance with an aspect of the present disclosure;

FIG. 14 is a second perspective view of the implant of FIG. 13, in accordance with an aspect of the present disclosure;

FIG. 15 is a first end view of the implant of FIG. 13, in accordance with an aspect of the present disclosure;

FIG. 16 is a second end view of the implant of FIG. 13, in accordance with an aspect of the present disclosure;

FIG. 17 is a first side view of the implant of FIG. 13, in accordance with an aspect of the present disclosure;

FIG. 18 is a second side view of the implant FIG. 13, in accordance with an aspect of the present disclosure;

FIG. 19 is a top view of the implant of FIG. 13, in accordance with an aspect of the present disclosure;

FIG. 20 is a bottom view of the implant of FIG. 13, in accordance with an aspect of the present disclosure;

FIG. 21 is a cross-sectional first end view of the implant of FIG. 13 taken along line 21-21 in FIG. 17, in accordance with an aspect of the present disclosure;

FIG. 22 is a cross-sectional second end view of the implant of FIG. 13 taken along line 22-22 in FIG. 18, in accordance with an aspect of the present disclosure;

FIG. 23 is a cross-sectional side view of the implant of FIG. 13 taken along line 23-23 in FIG. 15, in accordance with an aspect of the present disclosure;

FIG. 24 is a first perspective view of yet another implant, in accordance with an aspect of the present disclosure;

FIG. 25 is a second perspective view of the implant of FIG. 24, in accordance with an aspect of the present disclosure; and

FIG. 26 is a first end view of the implant of FIG. 24, in accordance with an aspect of the present disclosure;

FIG. 27 is a second end view of the implant of FIG. 24, in accordance with an aspect of the present disclosure;

FIG. 28 is a first side view of the implant of FIG. 24, in accordance with an aspect of the present disclosure;

FIG. 29 is a second side view of the implant of FIG. 24, in accordance with an aspect of the present disclosure;

FIG. 30 is a top view of the implant of FIG. 24, in accordance with an aspect of the present disclosure;

FIG. 31 is a bottom view of the implant of FIG. 24, in accordance with an aspect of the present disclosure;

FIG. 32 is a cross-sectional first end view of the implant of FIG. 24 taken along line 32-32 in FIG. 28, in accordance with an aspect of the present disclosure;

FIG. 33 is a cross-sectional second end view of the implant of FIG. 24 taken along line 33-33 in FIG. 29, in accordance with an aspect of the present disclosure;

FIG. 34 is a cross-sectional side view of the implant of FIG. 24 taken along line 34-34 in FIG. 26, in accordance with an aspect of the present disclosure;

FIG. 35 is a first perspective view of another implant, in accordance with an aspect of the present disclosure;

FIG. 36 is a second perspective view of the implant of FIG. 35, in accordance with an aspect of the present disclosure;

FIG. 37 is a first end view of the implant of FIG. 35, in accordance with an aspect of the present disclosure;

FIG. 38 is a second end view of the implant of FIG. 35, in accordance with an aspect of the present disclosure;

FIG. 39 is a first side view of the implant of FIG. 35, in accordance with an aspect of the present disclosure;

FIG. 40 is a second side view of the implant of FIG. 35, in accordance with an aspect of the present disclosure;

FIG. 41 is a top view of the implant of FIG. 35, in accordance with an aspect of the present disclosure;

FIG. 42 is a bottom view of the implant of FIG. 35, in accordance with an aspect of the present disclosure;

FIG. 43A is a cross-sectional first end view of the implant of FIG. 35 taken along line 43A-43A in FIG. 39, in accordance with an aspect of the present disclosure;

FIG. 43B is a cross-sectional second end view of the implant of FIG. 35 taken along line 43B-43B in FIG. 40, in accordance with an aspect of the present disclosure;

FIG. 44A is a cross-sectional first side view of the implant of FIG. 35 taken along line 44A-44A in FIG. 37, in accordance with an aspect of the present disclosure;

FIG. 44B is a cross-sectional second side view of the implant of FIG. 35 taken along line 44B-44B in FIG. 38, in accordance with an aspect of the present disclosure;

FIG. 45 is a perspective view of the implant of FIG. 35 including a lattice structure inserted into the openings of the implant of FIG. 35, in accordance with an aspect of the present disclosure;

FIG. 46 is a side view of a reamer, in accordance with an aspect of the present disclosure;

FIG. 47 is a bottom view of the reamer of FIG. 46, in accordance with an aspect of the present disclosure;

FIG. 48 is a first cross-sectional view of the reamer of FIG. 46 and a reamer driver coupled to the reamer, in accordance with an aspect of the present disclosure;

FIG. 49 is a second cross-sectional view of the reamer of FIG. 46 and a reamer driver coupled to the reamer, in accordance with an aspect of the present disclosure;

FIG. 50 is a top perspective view of two reamers of FIG. 46, in accordance with an aspect of the present disclosure;

FIG. 51 is a bottom perspective view of the reamer of FIG. 46, in accordance with an aspect of the present disclosure;

FIG. 52 is a first perspective view of an insertion instrument, in accordance with an aspect of the present disclosure;

FIG. 53 is a second perspective view of the insertion instrument of FIG. 52, in accordance with an aspect of the present disclosure;

FIG. 54 is a first side view of the insertion instrument of FIG. 52, in accordance with an aspect of the present disclosure;

FIG. 55 is a second side view of the insertion instrument of FIG. 52, in accordance with an aspect of the present disclosure;

FIG. 56 is an end view of the insertion instrument of FIG. 52, in accordance with an aspect of the present disclosure;

FIG. 57 is a top view of the insertion instrument of FIG. 52, in accordance with an aspect of the present disclosure;

FIG. 58 is a bottom view of the insertion instrument of FIG. 52, in accordance with an aspect of the present disclosure;

FIG. 59 is an exploded perspective view of the insertion instrument of FIG. 52, in accordance with an aspect of the present disclosure;

FIG. 60 is a first perspective view of the implant of FIG. 35 being inserted into a patient's ankle using the insertion instrument of FIG. 52, in accordance with an aspect of the present disclosure;

FIG. 61 is a lateral view of the implant of FIG. 35 implanted into the patient's ankle, in accordance with an aspect of the present disclosure;

FIG. 62 is a front view of the implant of FIG. 35 implanted into the patient's ankle, in accordance with an aspect of the present disclosure;

FIG. 63 is a first perspective view of the implant of FIG. 35 implanted into the patient's ankle, in accordance with an aspect of the present disclosure;

FIG. 64 is a top view of an insertion system including the insertion instrument of FIG. 52, an alignment member, a guide tube, and a bone screw, in accordance with an aspect of the present disclosure;

FIG. 65 is a first end view of the insertion system of FIG. 64, in accordance with an aspect of the present disclosure;

FIG. 66 is a perspective view of the insertion system of FIG. 64, in accordance with an aspect of the present disclosure;

FIG. 67 is an exploded, top view of the insertion instrument of FIG. 52, the alignment member of FIG. 64, and the guide tube of FIG. 64, in accordance with an aspect of the present disclosure;

FIG. 68 is an exploded, perspective view of the instruments of the insertion instrument of FIG. 67, in accordance with an aspect of the present disclosure;

FIG. 69 is an exploded, end view of the instruments of the insertion instrument of FIG. 67, in accordance with an aspect of the present disclosure;

FIG. 70 is a lateral view of the insertion system of FIG. 64 coupled to a patient's ankle and the implant of FIG. 35 inserted into the patient's ankle, in accordance with an aspect of the present disclosure;

FIG. 71 is an anterior view of patient's ankle of FIG. 70 with the insertion system of FIG. 64 and the implant of FIG. 35, in accordance with an aspect of the present disclosure;

FIG. 72 is a lateral perspective view of the patient's ankle of FIG. 70 with the insertion system of FIG. 64 and the implant of FIG. 35, in accordance with an aspect of the present disclosure;

FIG. 73 is an end view of the insertion system of FIG. 64 and the implant of FIG. 35, in accordance with an aspect of the present disclosure;

FIG. 74 is a first perspective view of the insertion system of FIG. 64 and the implant of FIG. 35, in accordance with an aspect of the present disclosure;

FIG. 75 is a second perspective view of the insertion system of FIG. 64 and the implant of FIG. 35, in accordance with an aspect of the present disclosure;

FIG. 76 is a bottom view of the insertion system of FIG. 64 and the implant of FIG. 35, in accordance with an aspect of the present disclosure;

FIG. 77 is a perspective view of the implant of FIG. 35 being inserted into a patient's ankle with the insertion system of FIG. 64, in accordance with an aspect of the present disclosure;

FIG. 78 is a medial view of FIG. 77, in accordance with an aspect of the present disclosure;

FIG. 79 is an anterior perspective view of FIG. 77, in accordance with an aspect of the present disclosure;

FIG. 80 is an image of the implant of FIG. 35 inserted into the patient's ankle, in accordance with an aspect of the present disclosure;

FIG. 81 is first perspective view of another implant, in accordance with an aspect of the present disclosure;

FIG. 82 is a second perspective view of the implant of FIG. 81, in accordance with an aspect of the present disclosure;

FIG. 83 is a first end view of the implant of FIG. 81, in accordance with an aspect of the present disclosure;

FIG. 84 is a second end view of the implant of FIG. 81, in accordance with an aspect of the present disclosure;

FIG. 85 is a first side view of the implant of FIG. 81, in accordance with an aspect of the present disclosure;

FIG. 86 is a second side view of the implant of FIG. 81, in accordance with an aspect of the present disclosure;

FIG. 87 is a top view of the implant of FIG. 81, in accordance with an aspect of the present disclosure;

FIG. 88 is a bottom view of the implant of FIG. 81, in accordance with an aspect of the present disclosure;

FIG. 89 is a cross-sectional view of the implant of FIG. 81 taken along line 89-89 in FIG. 85, in accordance with an aspect of the present disclosure;

FIG. 90 is a cross-sectional view of the implant of FIG. 81 taken along line 90-90 in FIG. 8, in accordance with an aspect of the present disclosure;

FIG. 91 is a first perspective view of yet another implant, in accordance with an aspect of the present disclosure;

FIG. 92 is a second perspective view of the implant of FIG. 91, in accordance with an aspect of the present disclosure;

FIG. 93 is a first end view of the implant of FIG. 91, in accordance with an aspect of the present disclosure;

FIG. 94 is a second end view of the implant of FIG. 91, in accordance with an aspect of the present disclosure;

FIG. 95 is a first side view of the implant of FIG. 91, in accordance with an aspect of the present disclosure;

FIG. 96 is a second side view of the implant of FIG. 91, in accordance with an aspect of the present disclosure;

FIG. 97 is a top view of the implant of FIG. 91, in accordance with an aspect of the present disclosure;

FIG. 98 is a bottom view of the implant of FIG. 91, in accordance with an aspect of the present disclosure;

FIG. 99 is a cross-sectional view of the implant of FIG. 91 taken along line 99-99 in FIG. 97, in accordance with an aspect of the present disclosure;

FIG. 100 is a cross-sectional view of the implant of FIG. 91 taken along line 100-100 in FIG. 98, in accordance with an aspect of the present disclosure;

FIG. 101 is a cross-sectional view of the implant of FIG. 91 taken along line 101-101 in FIG. 97, in accordance with an aspect of the present disclosure;

FIG. 102 is a cross-sectional view of the implant of FIG. 91 taken along line 102-102 in FIG. 98, in accordance with an aspect of the present disclosure;

FIG. 103 is a perspective view of the implant of FIG. 91 with a lattice structure within the implant, in accordance with an aspect of the present disclosure;

FIG. 104 depicts one embodiment of a surgical method for inserting an implant into a patient's ankle, in accordance with an aspect of the present disclosure;

FIG. 105 is a perspective view of an implant trial system in an anterior orientation, in accordance with an aspect of the present disclosure;

FIG. 106 is an enlarged front-side-top perspective view of a trial component of the implant trial system of FIG. 105, in accordance with an aspect of the present disclosure;

FIG. 107 is an enlarged back-side-bottom perspective view of the trial component view of FIG. 106, in accordance with an aspect of the present disclosure;

FIG. 108 is a front plan view of the trial component view of FIG. 106, in accordance with an aspect of the present disclosure;

FIG. 109 is a front plan cross-sectional view of the trial component view of FIG. 106, in accordance with an aspect of the present disclosure;

FIG. 110 is a side cross-sectional view of the trial component view of FIG. 106, in accordance with an aspect of the present disclosure;

FIG. 111 is a perspective view of an insertion instrument of the implant trial system of FIG. 105, in accordance with an aspect of the present disclosure;

FIG. 112 is a side view of the insertion instrument of FIG. 111, in accordance with an aspect of the present disclosure;

FIG. 113 is a front plan view of the insertion instrument of FIG. 111, in accordance with an aspect of the present disclosure;

FIG. 114 is a top or bottom view of the insertion instrument of FIG. 111, in accordance with an aspect of the present disclosure;

FIG. 115 is an anterior-lateral-proximal/top perspective view of an implant, insertion instrument and first anterior precision guide, in accordance with an aspect of the present disclosure;

FIG. 116 is an anterior view of the implant, the insertion instrument and the first anterior precision guide of FIG. 115, in accordance with an aspect of the present disclosure;

FIG. 117 is a medial side view of the implant, the insertion instrument and the first anterior precision guide of FIG. 115, in accordance with an aspect of the present disclosure;

FIG. 118 is a perspective exploded view of the first anterior precision guide of FIG. 115, in accordance with an aspect of the present disclosure;

FIG. 119 is a perspective view of a portion of the first anterior precision guide of FIG. 115, in accordance with an aspect of the present disclosure;

FIG. 120 is a lateral side view of the portion of the first anterior precision guide of FIG. 119, in accordance with an aspect of the present disclosure;

FIG. 121 is a medial side view of the portion of the first anterior precision guide of FIG. 119, in accordance with an aspect of the present disclosure;

FIG. 122 is an anterior view of the portion of the first anterior precision guide of FIG. 119, in accordance with an aspect of the present disclosure;

FIG. 123 is a posterior view of the portion of the first anterior precision guide of FIG. 119, in accordance with an aspect of the present disclosure;

FIG. 124 is a proximal/top plan view of the portion of the first anterior precision guide of FIG. 119, in accordance with an aspect of the present disclosure;

FIG. 125 is a bottom/distal view of the portion of the first anterior precision guide of FIG. 119, in accordance with an aspect of the present disclosure;

FIG. 126 is an anterior-lateral-proximal/top perspective view of an implant, insertion instrument and second anterior precision guide, in accordance with an aspect of the present disclosure;

FIG. 127 is an anterior view of the implant, the insertion instrument and the second anterior precision guide of FIG. 126, in accordance with an aspect of the present disclosure;

FIG. 128 is a medial side view of the implant, the insertion instrument and the second anterior precision guide of FIG. 126, in accordance with an aspect of the present disclosure;

FIG. 129 is a perspective exploded view of the second anterior precision guide of FIG. 126, in accordance with an aspect of the present disclosure;

FIG. 130 is a perspective view of a portion of the second anterior precision guide of FIG. 126, in accordance with an aspect of the present disclosure;

FIG. 131 is a lateral side view of the portion of the second anterior precision guide of FIG. 130, in accordance with an aspect of the present disclosure;

FIG. 132 is a medial side view of the portion of the second anterior precision guide of FIG. 130, in accordance with an aspect of the present disclosure;

FIG. 133 is an anterior view of the portion of the second anterior precision guide of FIG. 130, in accordance with an aspect of the present disclosure;

FIG. 134 is a posterior view of the portion of the second anterior precision guide of FIG. 130, in accordance with an aspect of the present disclosure;

FIG. 135 is a proximal/top plan view of the portion of the second anterior precision guide of FIG. 130, in accordance with an aspect of the present disclosure;

FIG. 136 is a bottom/distal view of the portion of the second anterior precision guide of FIG. 130, in accordance with an aspect of the present disclosure;

FIG. 137 is a perspective view of an implant trial system in a lateral orientation, in accordance with an aspect of the present disclosure;

FIG. 138 is an anterior-lateral-proximal/top perspective view of an implant, insertion instrument and first lateral precision guide, in accordance with an aspect of the present disclosure;

FIG. 139 is an anterior view of the implant, the insertion instrument and the first lateral precision guide of FIG. 138, in accordance with an aspect of the present disclosure;

FIG. 140 is a lateral side view of the implant, the insertion instrument and the first lateral precision guide of FIG. 138, in accordance with an aspect of the present disclosure;

FIG. 141 is an anterior-lateral-proximal/top perspective view of an implant, insertion instrument and second lateral precision guide, in accordance with an aspect of the present disclosure;

FIG. 142 is an anterior view of the implant, the insertion instrument and the second lateral precision guide of FIG. 141, in accordance with an aspect of the present disclosure;

FIG. 143 is a lateral side view of the implant, the insertion instrument and the second lateral precision guide of FIG. 141, in accordance with an aspect of the present disclosure;

FIG. 144 is an elevational perspective exploded view of the implant, the insertion instrument and the second lateral precision guide of FIG. 141, in accordance with an aspect of the present disclosure;

FIG. 145 is a perspective view of a portion of the second lateral precision guide of FIG. 141, in accordance with an aspect of the present disclosure;

FIG. 146 is a lateral side view of the portion of the second lateral precision guide of FIG. 145, in accordance with an aspect of the present disclosure;

FIG. 147 is a medial side view of the portion of the second lateral precision guide of FIG. 145, in accordance with an aspect of the present disclosure;

FIG. 148 is an anterior view of the portion of the second lateral precision guide of FIG. 145, in accordance with an aspect of the present disclosure;

FIG. 149 is a posterior view of the portion of the second lateral precision guide of FIG. 145, in accordance with an aspect of the present disclosure;

FIG. 150 is a proximal/top plan view of the portion of the second lateral precision guide of FIG. 145, in accordance with an aspect of the present disclosure;

FIG. 151 is a bottom/distal view of the portion of the second lateral precision guide of FIG. 145, in accordance with an aspect of the present disclosure;

FIG. 152 is a perspective view of a posterior implant trial system, in accordance with an aspect of the present disclosure;

FIG. 153 is a posterior-medial-proximal/top perspective view of an implant, insertion instrument and first posterior precision guide, in accordance with an aspect of the present disclosure;

FIG. 154 is a posterior view of the implant, the insertion instrument and the first posterior precision guide of FIG. 153, in accordance with an aspect of the present disclosure;

FIG. 155 is a lateral side view of the implant, the insertion instrument and the first posterior precision guide of FIG. 153, in accordance with an aspect of the present disclosure;

FIG. 156 is a posterior-medial-proximal/top perspective view of an implant, insertion instrument and second posterior precision guide, in accordance with an aspect of the present disclosure;

FIG. 157 is a posterior view of the implant, the insertion instrument and the second posterior precision guide of FIG. 156, in accordance with an aspect of the present disclosure;

FIG. 158 is a lateral side view of the implant, the insertion instrument and the second posterior precision guide of FIG. 156, in accordance with an aspect of the present disclosure;

FIG. 159 is an elevational perspective exploded view of the implant, the insertion instrument and the second posterior precision guide of FIG. 156, in accordance with an aspect of the present disclosure;

FIG. 160 is a perspective view of a portion of the second posterior precision guide of FIG. 156, in accordance with an aspect of the present disclosure;

FIG. 161 is a posterior view of the portion of the second posterior precision guide of FIG. 160, in accordance with an aspect of the present disclosure;

FIG. 162 is an anterior view of the portion of the second posterior precision guide of FIG. 160, in accordance with an aspect of the present disclosure;

FIG. 163 is a lateral side view of the portion of the second posterior precision guide of FIG. 160, in accordance with an aspect of the present disclosure;

FIG. 164 is a medial view of the portion of the second posterior precision guide of FIG. 160, in accordance with an aspect of the present disclosure;

FIG. 165 is a bottom/distal plan view of the portion of the second posterior precision guide of FIG. 160, in accordance with an aspect of the present disclosure;

FIG. 166 is a proximal/top view of the portion of the second posterior precision guide of FIG. 160, in accordance with an aspect of the present disclosure;

FIG. 167 is a front-side-top perspective view of another trial component of an implant trial system, in accordance with an aspect of the present disclosure;

FIG. 168 is a back-side-bottom perspective view of the trial component view of FIG. 167, in accordance with an aspect of the present disclosure;

FIG. 169 is a front plan view of the trial component view of FIG. 167, in accordance with an aspect of the present disclosure;

FIG. 170 is a side view of the trial component view of FIG. 167, in accordance with an aspect of the present disclosure;

FIG. 171 is a front plan cross-sectional view of the trial component view of FIG. 167, in accordance with an aspect of the present disclosure; and

FIG. 172 is a side cross-sectional view of the trial component view of FIG. 167, in accordance with an aspect of the present disclosure.

### DETAILED DESCRIPTION FOR CARRYING OUT THE INVENTION

Generally stated, disclosed herein are implants, instruments, and systems for correcting bone deformities. Further, surgical methods for using the implants, instruments and systems to correct bone deformities are discussed.

In this detailed description and the following claims, the words proximal, distal, anterior or plantar, posterior or dorsal, medial, lateral, superior and inferior are defined by their standard usage for indicating a particular part or portion of a bone or instrument according to the relative disposition of the natural bone or directional terms of reference. For example, “proximal” means the portion of a device or instrument nearest the torso, while “distal” indicates the portion of the device or instrument farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above, and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, instruments, systems and methods are described herein with reference to use with the bones of the foot, ankle and lower leg may be used to describe the surfaces, positions, directions or orientations of the implants, instruments, systems and methods. Further, the implants, devices, instrumentation, systems, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, systems, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, systems, and methods, and the aspects, components, features and the like thereof, described herein with respect to the right foot may be mirrored so that they likewise function with the left foot. Further, the implants, devices, instrumentation, systems and methods, and the aspects, components, features and the like thereof, disclosed herein are described with respect to the foot and ankle for brevity purposes, but it should be understood that the implants, devices, instrumentation, systems and methods may be used with other bones and/or joints of the body having similar structures.

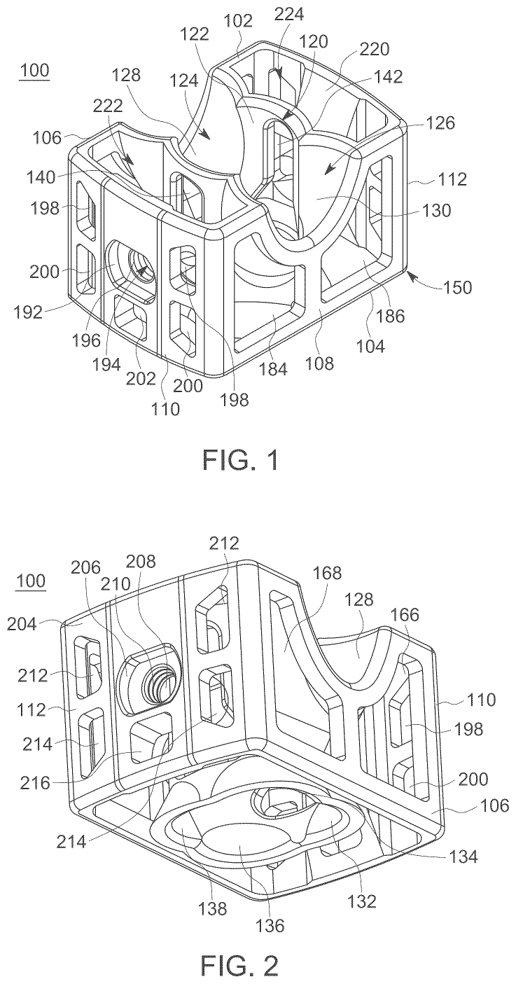

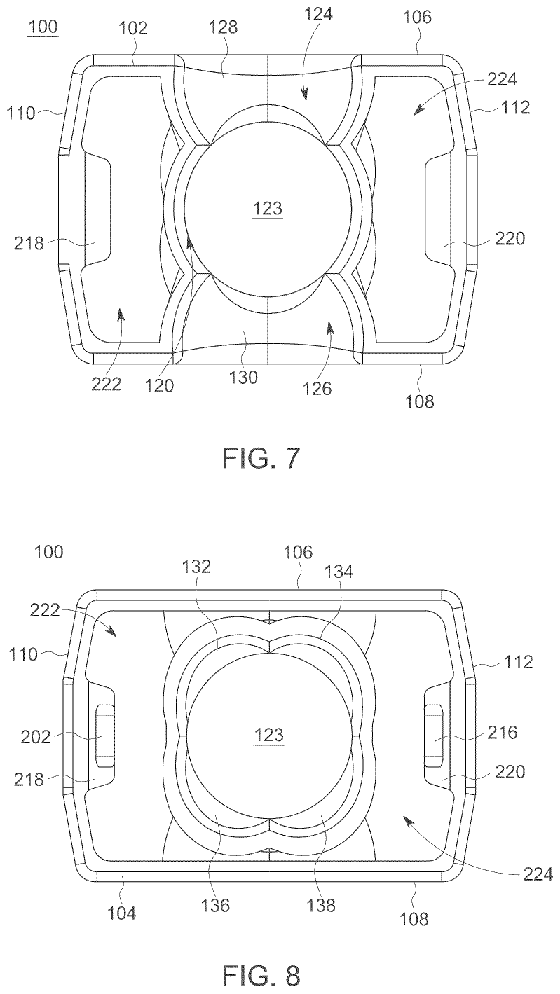

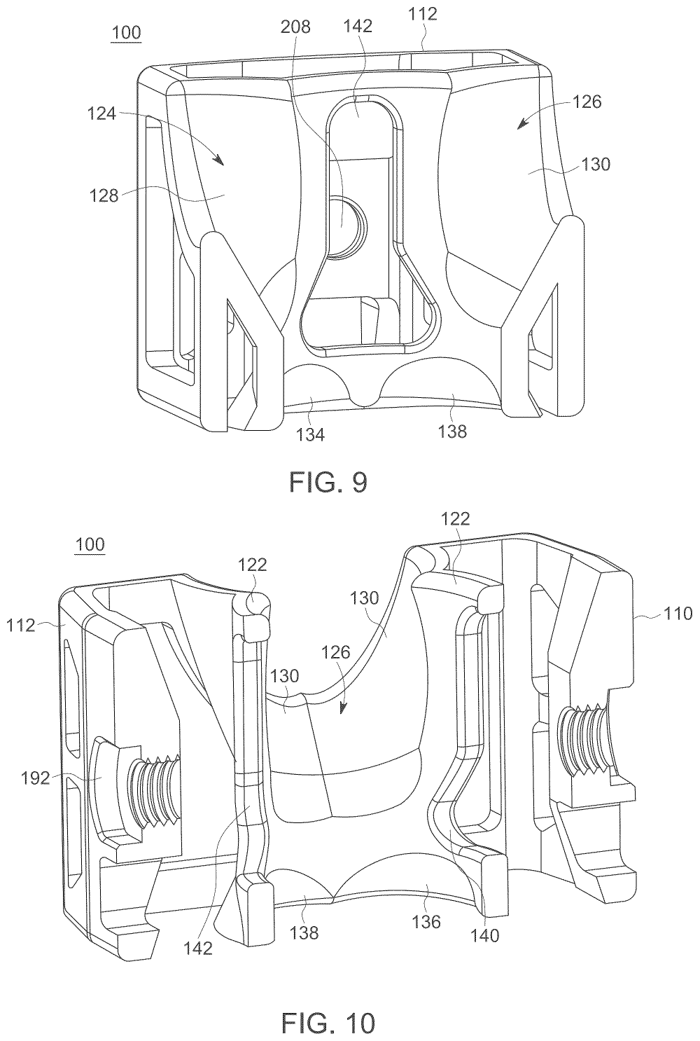

Referring to the drawings, wherein like reference numerals are used to indicate like or analogous components throughout the several views, and with particular reference to FIGS. 1-12, there is illustrated one embodiment of an implant 100. The implant 100 may include a first side or top side 102, a second side or bottom side 104, a third side or right side 106, a fourth side or left side 108, a fifth side or first end 110, and a sixth side or second end 112. The first side 102 may extend, for example, generally parallel to the second side 104. The third side 106 may extend, for example, generally parallel to the fourth side 108. The fifth side 110 may extend, for example, generally parallel to the sixth side 112. The implant 100 may also include an inner body 120 surrounded by a frame or housing 150.

With continued reference to FIGS. 1-12, the inner body 120 may include an intermediate portion 122 extending through the implant 100 from the first side 102 to the second side 104. The intermediate portion 122 may form a through hole 123 extending through the inner body 120, as shown in FIGS. 1, 2, and 5-11. The inner body 120 may also include a first guide hole or first access aperture 124 and a second guide hole or second access aperture 126. The first guide hole 124 may be, for example, partially recessed into the first side 102 and the third side 106 of the implant 100 and extend through the implant 100 from the first side 102 to the second side 104 on at least one angle. Similarly, the second guide hole 126 may be, for example, partially recessed into the first side 102 and the fourth side 108 of the implant 100 and extend through the implant 100 from the first side to the second side 104 on at least one angle.

Referring now to FIGS. 1, 2, and 7-10, the first guide hole 124 may include at least one first upper portion 128, a first lower portion 132, and a second lower portion 134. As shown in FIGS. 1, 2, and 7-10, the second guide hole 126 may include at least one second upper portion 130, a third lower portion 136, and a fourth lower portion 138.

The at least one first upper portion 128 may be, for example, configured or sized and shaped to allow for a fastener (not shown) to be inserted through the first upper portion 128 of the first guide hole 124 at more than one angle. For example, the more than one angle of insertion of may be at least one first angle that forms at least one first path from the first upper portion 128 to the third lower portion 136 or at least one second angle that forms at least one second path from the first upper portion 128 to the fourth lower portion 138.

The at least one second upper portion 130 may be, for example, configured or sized and shaped to allow for a fastener (not shown) to be inserted through the second upper portion 130 of the second guide hole 126 at more than one angle. For example, the more than one angle may be at least one first angle that forms at least one first path from the second upper portion 130 to the first lower portion 132 or at least one second angle that forms at least one second path from the second upper portion 130 to the second lower portion 134.

With continued reference to FIGS. 1, 9, and 10, the intermediate portion 122 of the inner body 120 may also include at least one first channel or slot 140 positioned on a first side of the intermediate portion 122 and at least one second channel or slot 142 positioned on a second side of the intermediate portion 122. The at least one first channel 140 may be positioned, for example, opposite the at least one second channel 142. In addition, the first and second channels 140, 142 may be positioned between the first guide hole 124 and the second guide hole 126. The first channel 140 may be, for example, shaped such that a first portion near the first side 102 of the implant 100 has a smaller width than a second portion near the second side 104 of the implant 100. The second channel 142 may also be, for example, shaped such that a first portion near the first side 102 of the implant 100 has a smaller width than a second portion near the second side 104 of the implant 100. The first portion of the first and second channels 140, 142 may be, for example, generally rectangular or oval shaped and the second portion of the first and second channels 140, 142 may be, for example, generally triangularly shaped. The first portion of the channels 140, 142 extends into the second portion of the channels 140, 142.

The frame 150 may include supports 152, 154, 156, 158, 160, 162, 164 on the third side 106 of the implant 100, as shown in FIG. 6. The first support, first base or first linear member 152 may be positioned, for example, at an inferior end of the third side 106, i.e. where the third side 106 engages the second side 104. The first arm, second linear member or second support 154 may extend from a first end of the first support 152, for example, perpendicular to the first support 152. The first arm 154 may be positioned, for example, where the third side 106 engages the fifth side 110. The second arm, third linear member or third support 156 may extend from a second end of the first support 152, for example, perpendicular to the first support 152. The fourth linear member or fourth support 158 may extend from a first end of the first arm 154 toward the first guide hole 124 of the intermediate portion 122. The fifth linear member or fifth support 160 may extend from a first end of the second arm 156 toward the first guide hole 124 of the intermediate portion 122. The first guide hole member or sixth support 162 may, for example, extend between the fourth linear member 158 and the fifth linear member 160. The first guide hole member 162 may be, for example, coupled to or integral with an exterior surface of the first upper portion 128 of the first guide hole 124 of the intermediate portion 122. The first guide hole member 162 may be, for example, curved. The curvature of the first guide hole member 162 may be, for example, a concave curvature. The third arm, sixth linear member or seventh support 164 may be coupled to an intermediate portion of the first guide hole member 162 on a first end and the first base 152 on a second end. The third arm 164 may, for example, couple to the distal most portion of the first guide hole member 162. The first arm 154, second arm 156 and third arm 164 may each, for example, extend parallel to each other as the arms 154, 156, 164 extend from the bottom side 104 toward the top side 102. The third side 106 may also include at least one first window 166 and at least one second window 168, as shown in FIGS. 2 and 6. The at least one first window 166 may be positioned, for example, intermediate a portion of the first base 152, first arm 154, fourth linear member 158, a portion of the first guide hole member 162, and the third arm 164. Thus, the first base 152, first arm 154, fourth linear member 158, a portion of the first guide hole member 162, and the third arm 164 provide the surrounding structure to form the at least one first window 166 extending through the third side 106. The at least one second window 168 may be positioned, for example, intermediate a portion of the first base 152, the second arm 156, fifth linear member 160, a portion of the first guide hole member 162, and the third arm 164. Thus, the first base 152, the second arm 156, the fifth linear member 160, a portion of the first guide hole member 162, and the third arm 164 provide the surrounding structure to form the at least one second window 168 extending through the third side 106.

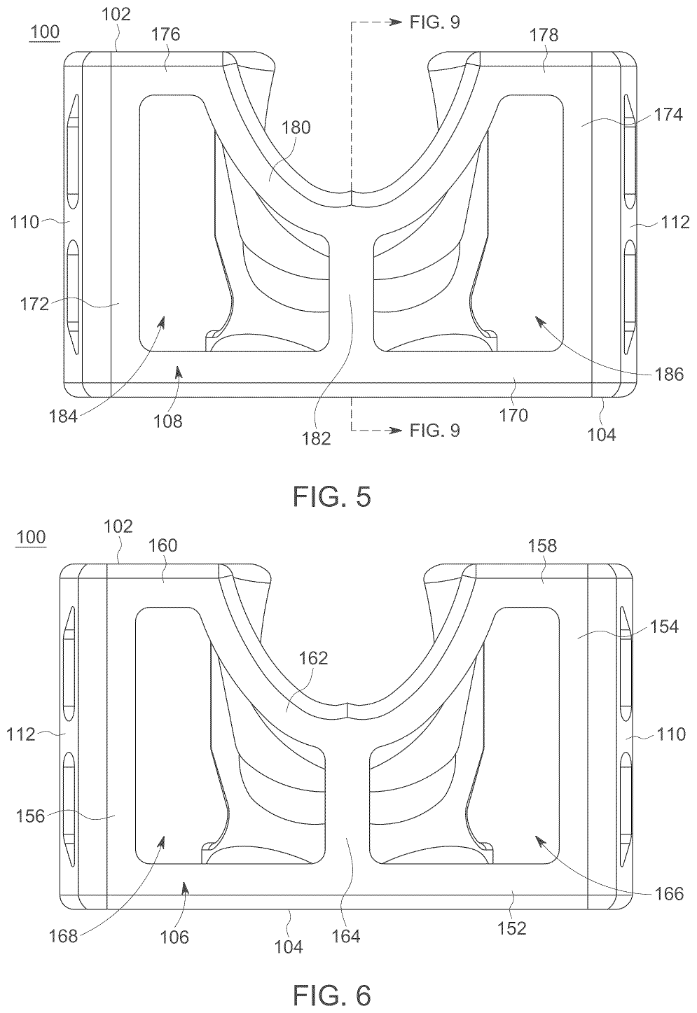

Referring now to FIG. 5, the frame 150 may also include supports 170, 172, 174, 176, 178, 180, 182 on the fourth side 108 of the implant 100. The eighth support, second base or seventh linear member 170 may be positioned, for example, at an inferior end of the fourth side 108, i.e. where the fourth side 108 engages the second side 104. The fourth arm, eighth linear member or ninth support 172 may extend from a first end of the eighth support 170, for example, perpendicular to the eighth support 170. The fourth arm 172 may be positioned, for example, where the fourth side 108 engages the fifth side 110. The fifth arm, ninth linear member or tenth support 174 may extend from a first end of the second base 170, for example, perpendicular to the second base 170. The tenth linear member or eleventh support 176 may extend from a first end of the fourth arm 172 toward the second guide hole 126 of the intermediate portion 122. The eleventh linear member or twelfth support 176 may extend from a first end of the fourth arm 172 toward the second guide hole 126 of the intermediate portion 122. The second guide hole member or thirteenth support 180 may, for example, extend between the tenth linear member 176 and the eleventh linear member 178. The second guide hole member 180 may be, for example, coupled to or integral with an exterior surface of the second upper portion 130 of the second guide hole 126 of the intermediate portion 122. The second guide hole member 180 may be, for example, curved. The curvature of the second guide hole member 180 may be, for example, a concave curvature. The sixth arm, twelfth linear member or fourteenth support 182 may be coupled to an intermediate portion of the second guide hole member 180 on a first end and the second base 170 on a second end. The sixth arm 182 may, for example, couple to the distal most portion of the second guide hole member 180. The fourth arm 172, fifth arm 174 and sixth arm 182 may each, for example, extend parallel to each other as the arms 172, 174, 182 extend from the bottom side 104 toward the top side 102. The fourth side 108 may also include at least one third window 184 and at least one fourth window 186, as shown in FIGS. 1 and 5. The at least one third window 184 may be positioned, for example, intermediate a portion of the second base 170, the fourth arm 172, the tenth linear member 176, a portion of the second guide hole member 180, and the sixth arm 182. The at least one fourth window 186 may be positioned, for example, intermediate a portion of the second base 170, the fifth arm 174, the eleventh linear member 178, a portion of the second guide hole member 180, and the sixth arm 182.

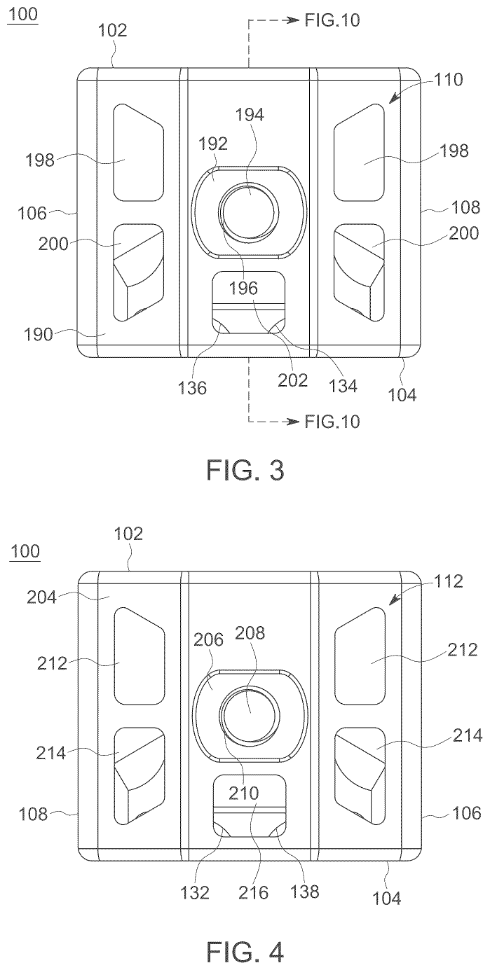

Referring now to FIG. 3, the frame 150 may include a first support member 190 on the fifth side 110 of the implant 100. The first support member 190 may include a first recessed region 192 extending into an intermediate portion from the fifth side 110. The first recessed region 192 may have, for example, a length, width and height and the length may be larger than the height. The height may extend in a direction between the top side 102 and the bottom side 104, the length may extend in a direction between the third side 106 and the fourth side 108, and the width may extend in a direction between the first end 110 and the second end 112. The first support member 190 may also include a first through hole 194 extending through the first support member 190. The first through hole 194 may extend, for example, entirely through the fifth side 110 of the implant 100. The first through hole 194 may include, for example, threads 196 on the interior surface. The first support member 190 may also include at least one fifth window, superior window, or opening 198 extending through the first support member 190. The at least one fifth window 198 may include, for example, two parallel sides, a base portion connecting the two parallel sides at a first end, and a top portion connecting the two parallel sides at a second end. The top portion may be, for example, angled as it extends between the two sides. The at least one fifth window 198 may be, for example, at least two windows 198 positioned on a medial and lateral side of the fifth side 110. As shown in the depicted embodiment, the two fifth windows 198 may be positioned to be mirror images of each other and near a superior end the implant 100. The first support member 190 may also include at least one sixth window, inferior window, or opening 200 extending through the first support member 190. The at least one sixth window 200 may include, for example, two parallel sides, a base portion connecting the two parallel sides, and a top portion connecting the two parallel sides. The base portion may be, for example, angled as it extends between the two sides. The at least one sixth window 200 may be, for example, at least two windows 200 positioned on a medial and lateral side of the fifth side 110. As shown in the depicted embodiment, the two sixth windows 200 may be positioned to be mirror images of each other and near an inferior end of the fifth side 110 of the implant 100. The first support member 190 may also include at least one seventh window, intermediate window or opening 202. The at least one seventh window 202 may be positioned, for example, adjacent to the first recessed region 192. In the depicted embodiment, the at least one seventh window 202 is one seventh window 202 that is positioned inferior to the recessed region 192 and between the recessed region 192 and the bottom side 104.

Referring now to FIG. 4, the frame 150 may include a second support member 204 on the sixth side 112 of the implant 100. The second support member 204 may include a second recessed region 206 extending into an intermediate portion from the sixth side 112. The second recessed region 206 may have, for example, a length, width and height and the length may be larger than the height. The height may extend in a direction between the top side 102 and the bottom side 104, the length may extend in a direction between the third side 106 and the fourth side 108, and the width may extend in a direction between the first end 110 and the second end 112. The second support member 204 may also include a second through hole 208 extending through the second support member 204. The second through hole 208 may extend, for example, entirely through the sixth side 112 of the implant 100. The second through hole 208 may include, for example, threads 210 positioned on the interior surface of the second through hole 208. The second support member 204 may also include at least one eighth window, superior window, or opening 212 extending through the second support member 204. The at least one eighth window 212 may include, for example, two parallel sides, a base portion connecting the two parallel sides at a first end, and a top portion connecting the two parallel sides at a second end. The top portion may be, for example, angled as it extends between the two sides. The at least one eighth window 212 may be, for example, at least two windows 212 positioned on a medial and lateral side of the sixth side 112. As shown in the depicted embodiment, the two eighth windows 212 may be positioned to be mirror images of each other and near a superior end the implant 100. The second support member 204 may also include at least one ninth window, inferior window, or opening 214 extending through the second support member 204. The at least one ninth window 214 may include, for example, two parallel sides, a base portion connecting the two parallel sides, and a top portion connecting the two parallel sides. The base portion may be, for example, angled as it extends between the two sides. The at least one ninth window 214 may be, for example, at least two windows 214 positioned on a medial and lateral side of the sixth side 112. As shown in the depicted embodiment, the two ninth windows 214 may be positioned to be mirror images of each other and near an inferior end of the sixth side 112 of the implant 100. The second support member 204 may also include at least one tenth window, intermediate window or opening 216. The at least one tenth window 216 may be positioned, for example, adjacent to the second recessed region 206. In the depicted embodiment, the at least one tenth window 216 is, for example, one tenth window 216 that is positioned inferior to the second recessed region 206 and between the second recessed region 206 and the bottom side 104.

Referring now to FIGS. 1, 7, 8 and 10, the frame 150 may also include a first protrusion or interior protrusion 218 extending away from an interior surface of the fifth side 110. The first protrusion 218 may be positioned, for example, intermediate the third side 106 and the fourth side 108. The top surface of the first protrusion 218 may be, for example, angled as the top surface extends from the first side 102 toward the second side 104, as shown in FIGS. 1 and 10. The bottom surface of the first protrusion 218 may also be, for example, angled as the bottom surface extends from the second side 104 toward the first side 102. The first through hole 194 may extend through the first protrusion 218.

Referring now to FIGS. 2, 7, 8 and 10, the frame 150 may include a second protrusion or interior protrusion 220 extending away from an interior surface of the sixth side 112. The second protrusion 220 may be positioned, for example, intermediate the third side 106 and the fourth side 108. The top surface of the second protrusion 220 may be, for example, angled as the top surface extends from the first side 102 toward the second side 104, as shown in FIGS. 2 and 10. The bottom surface of the second protrusion 220 may also be, for example, angled as the bottom surface extends from the second side 104 toward the first side 102. The second through hole 208 may extend through the second protrusion 220.

With continued reference to FIGS. 1, 2, and 7-11, the implant 100 may also include a first passageway 222 extending between the frame 150 and the inner body 120 near the first end 110. The first passageway 222 is open from the first side 102 to the second side 104. The first through hole 194 and the openings 198, 200, 202 extend from the exterior surface of the frame 150 through to the passageway 222. The implant 100 may further include a second passageway 224 extending between the frame 150 and the inner body 120 near the second end 112. The second passageway 224 is open from the first side 102 to the second side 104. The second through hole 208 and the openings 212, 214, 216 extend from the exterior surface of the frame 150 through to the passageway 224.

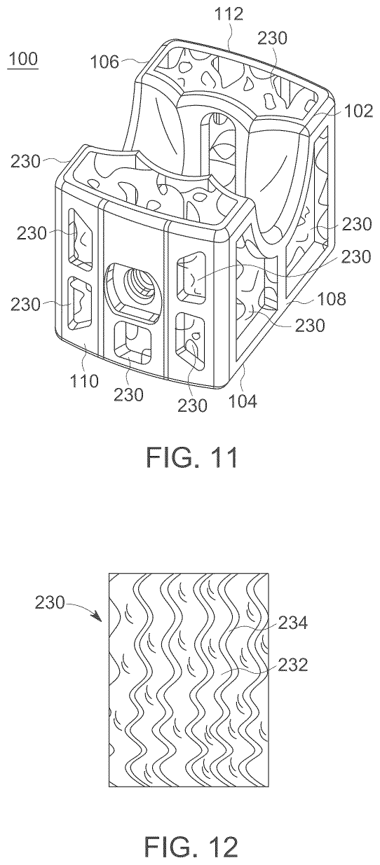

As shown in FIGS. 11 and 12, the implant 100 may also include a lattice structure 230 positioned within the openings 166, 168, 184, 186, 198, 200, 202, 212, 214, 216, 222, 224 of the implant 100. The lattice structure 230 may be positioned, for example, within the first and second passageways 222, 224 to assist with bone fusion. The lattice structure 230 may, for example, include a plurality of recessed regions or grooves 232 between a plurality of raised portions 234, as shown in magnified FIG. 12. Alternatively, the lattice structure may be, for example, printed within the passageways 222, 224. The lattice structure may be, for example, non-nodal randomized web based matrix. The lattice structure matrix may have a pore size of, for example, approximately 4 mm to approximately 5 mm. The lattice structure may include, for example, rough elements that span between the structure that forms the passageways 222, 224. The lattice structure 230 may be, for example, offset from the outer frame. The lattice structure 230 may be offset inside of the frame approximately 0.5 mm. Thus, the frame of the implant 100 is the only bone contacting element once implanted and the lattice structure 230 does not directly contact bone.

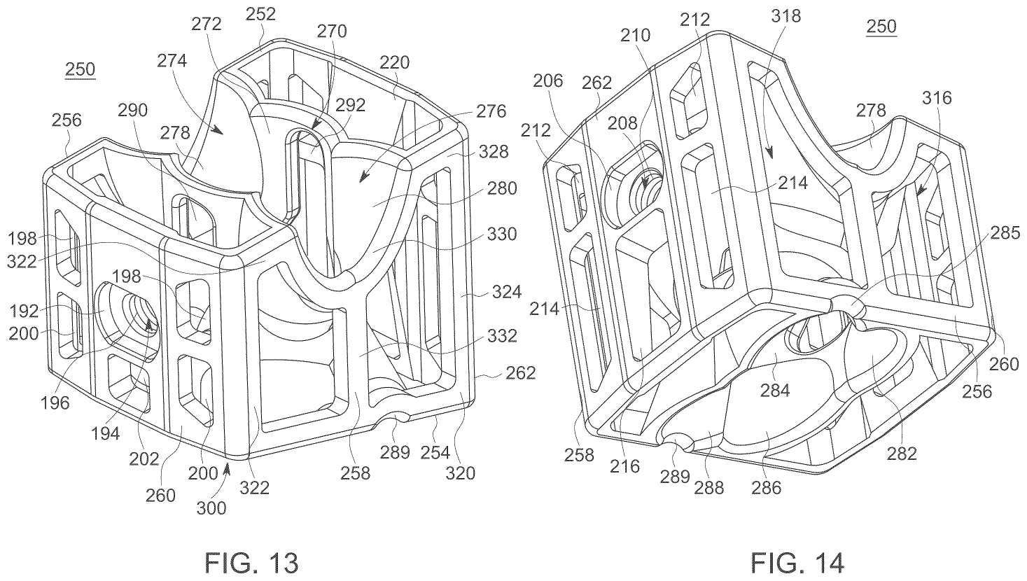

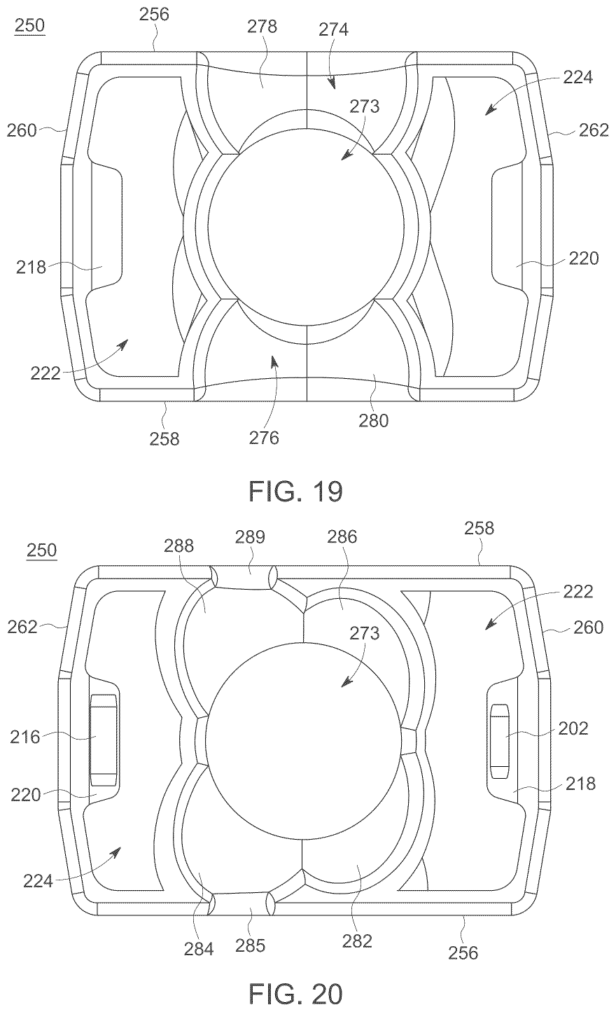

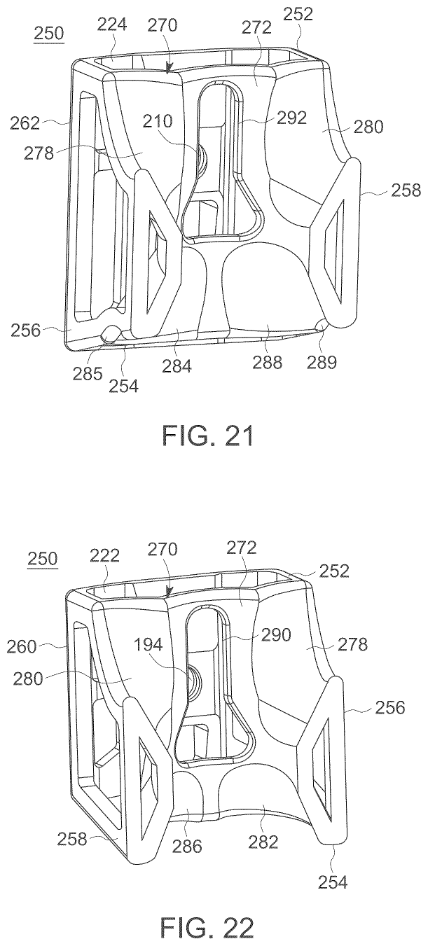

Referring now to FIGS. 13-23, another implant 250 is shown. The implant 250 may include a first side or top side 252, a second side or bottom side 254, a third side or right side 256, a fourth side or left side 258, a fifth side or first end 260, and a sixth side or second end 262. The second side 254 may be, for example, angled relative to the first side 252 as the first and second sides 252, 254 extend between the first end 260 and the second end 262. The third side 256 may extend, for example, generally parallel to the fourth side 258. The fifth side 260 may extend, for example, generally parallel to the sixth side 262. The height of fifth side 260 is, for example, smaller than the height of the sixth side 262. The heights may be measure between the top side 252 and the bottom side 254. The implant 250 may also include an inner body 270 surrounded by a frame or housing 300.

With continued reference to FIGS. 13-23, the inner body 270 may include an intermediate portion 272 extending through the implant 250 from the first side 252 to the second side 254. The intermediate portion 272 may form a through hole 273 extending through the inner body 270, as shown in FIGS. 13, 14, and 17-23. The inner body 270 may also include a first guide hole or first access aperture 274 and a second guide hole or second access aperture 276. The first guide hole 274 may be, for example, partially recessed into the first side 252 and the third side 256 of the implant 250 and extend through the implant 250 from the first side 252 to the second side 254 on at least one angle. Similarly, the second guide hole 276 may be, for example, partially recessed into the first side 252 and the fourth side 258 of the implant 250 and extend through the implant 250 from the first side 252 to the second side 254 on at least one angle.

Referring now to FIGS. 1, 2, and 7-10, the first guide hole or access aperture 274 may include at least one first upper portion 278, a first lower portion 282, and a second lower portion 284. The second lower potion 284 may also include, for example, a first recess 285 inset into the second lower potion 284 from the bottom side 254. As shown in FIGS. 1, 2, and 7-10, the second guide hole or access aperture 276 may include at least one second upper portion 280, a third lower portion 286, and a fourth lower portion 288. The fourth lower potion 288 may also include, for example, a second recess 289 inset into the fourth lower portion 288 from the bottom side 254.

The at least one first upper portion 278 may be, for example, configured or sized and shaped to allow for a fastener (not shown) to be inserted through the first upper portion 278 of the first guide hole 274 at more than one angle. For example, the more than one angle may be at least one first angle that forms at least one first path from the first upper portion 278 to the third lower portion 286 or at least one second angle that forms at least one second path from the first upper portion 278 to the fourth lower portion 288.

The at least one second upper portion 280 may be, for example, configured or sized and shaped to allow for a fastener (not shown) to be inserted through the second upper portion 280 of the second guide hole 276 at more than one angle. For example, the more than one angle may be at least one first angle that forms at least one first path from the second upper portion 280 to the first lower portion 282 or at least one second angle that forms at least one second path from the second upper portion 280 to the second lower portion 284.

With continued reference to FIGS. 1, 9, and 10, the intermediate portion 272 of the inner body 270 may also include at least one first channel or slot 290 positioned on a first side of the intermediate portion 272 and at least one second channel or slot 292 positioned on a second side of the intermediate portion 272. The at least one first channel 290 may be positioned, for example, opposite the at least one second channel 292. In addition, the first and second channels 290, 292 may be positioned between the first guide hole 274 and the second guide hole 276. The first channel 290 may be, for example, shaped such that a first portion near the first side 252 of the implant 250 has a smaller width than a second portion near the second side 254 of the implant 250. The second channel 292 may also be, for example, shaped such that a first portion near the first side 252 of the implant 250 has a smaller width than a second portion near the second side 254 of the implant 250. The first portion of the first and second channels 290, 292 may be, for example, generally rectangular or oval shaped and the second portion of the first and second channels 290, 292 may be, for example, generally triangularly shaped. The first portion of the channels 290, 292 extends into the second portion of the channels 290, 292.

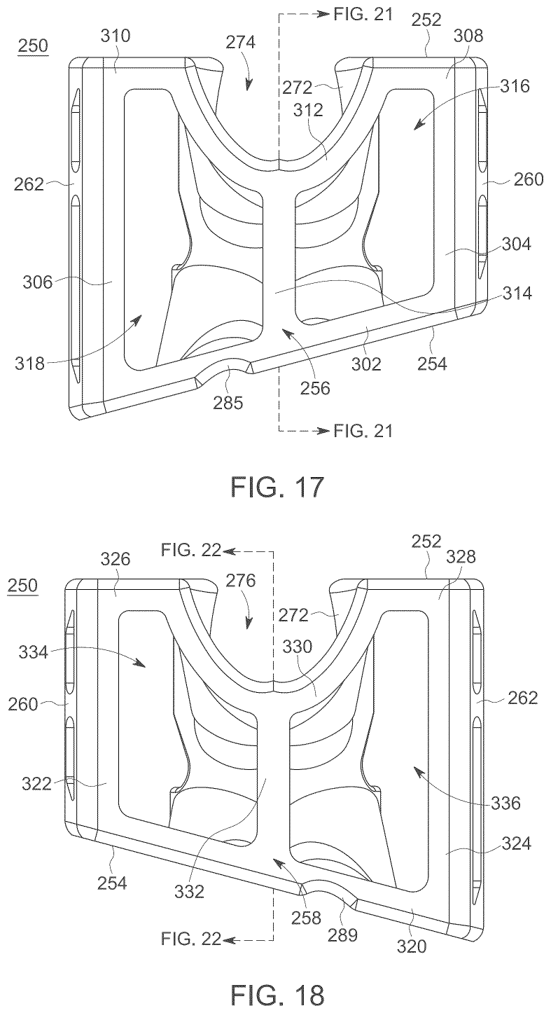

The frame 300 may include supports 302, 304, 306, 308, 310, 312, 314 on the third side 256 of the implant 250, as shown in FIG. 17. The first support, first base or first linear member 302 may be positioned, for example, at an inferior end of the third side 256, i.e. where the third side 256 engages the second side 254. The first arm, second linear member or second support 304 may extend from a first end of the first support 302. The first arm 304 may be positioned, for example, where the third side 256 engages the fifth side 260. The second arm, third linear member or third support 306 may extend from a second end of the first support 302. The first arm 304 may be positioned, for example, parallel to the second arm 306 as the arms 304, 306 extend from the first support 302 toward the top side 252 of the implant 250. The fourth linear member or fourth support member 308 may extend from a first end of the first arm 304 toward the first guide hole 274 of the intermediate portion 272. The fifth linear member or fifth support 310 may extend from a first end of the second arm 306 toward the first guide hole 274 of the intermediate portion 272. The first guide hole member or sixth support 312 may, for example, extend between the fourth linear member 308 and the fifth linear member 310. The first guide hole member 312 may be, for example, coupled to or integral with an exterior surface of the first upper portion 278 of the first guide hole 274 of the intermediate portion 272. The first guide hole member 312 may be, for example, curved. The curvature of the first guide hole member 312 may be, for example, a concave curvature. The third arm, sixth linear member or seventh support 314 may be coupled to an intermediate portion of the first guide hole member 312 on a first end and the first base 302 on a second end. The first arm 304, second arm 306 and third arm 314 may each, for example, extend parallel to each other. The third side 256 may also include at least one first window 316 and at least one second window 318, as shown in FIGS. 14 and 17. The at least one first window 316 may be positioned, for example, intermediate a portion of the first base 302, the first arm 304, the fourth linear member 308, a portion of the first guide hole member 312, and the third arm 314. The at least one second window 318 may be positioned, for example intermediate a portion of the first base 302, the second arm 306, fifth linear member 310, a portion of the first guide hole member 312, and the third arm 314.

Referring now to FIG. 18, the frame 300 may also include supports 320, 322, 324, 326, 328, 330, 332 on the fourth side 258 of the implant 250. The eighth support, second base or seventh linear member 320 may be positioned, for example, at an inferior end of the fourth side 258, i.e. where the fourth side 258 engages the second side 254. The second base 320 may be, for example, angled between the first end 260 and the second end 262. The fourth arm, eighth linear member or ninth support 322 may extend from a first end of the second base 320. The fourth arm 322 may be positioned, for example, where the fourth side 258 engages the fifth side 260. The fifth arm, ninth linear member or tenth support 324 may extend from a second end of the of the second base 320. The tenth linear member or fourth support member 326 may extend from a first end of the fourth arm 322 toward the second guide hole 276 of the intermediate portion 272. The eleventh linear member or twelfth support 328 may extend from a first end of the fifth arm 324 toward the second guide hole 276 of the intermediate portion 272. The second guide hole member or thirteenth support 330 may, for example, extend between the tenth linear member 326 and the eleventh linear member 328. The second guide hole member 330 may be, for example, coupled to or integral with an exterior surface of the second upper portion 280 of the second guide hole 276 of the intermediate portion 272. The second guide hole member 330 may be, for example, curved. The curvature of the second guide hole member 330 may be, for example, a concave curvature. The sixth arm, twelfth linear member or fourteenth support 332 may be coupled to an intermediate portion of the second guide hole member 330 on a first end and the second base 320 on a second end. The fourth arm 322, fifth arm 324 and sixth arm 332 may each, for example, extend parallel to each other. The fourth side 258 may also include at least one third window 334 and at least one fourth window 336, as shown in FIGS. 13 and 18. The at least one third window 334 may be positioned, for example, intermediate a portion of the second base 320, fourth arm 322, tenth linear member 326, a portion of the second guide hole member 330, and the sixth arm 332. The at least one fourth window 336 may be positioned, for example intermediate a portion of the second base 320, the fifth arm 324, eleventh linear member 328, a portion of the second guide hole member 330, and the sixth arm 332.

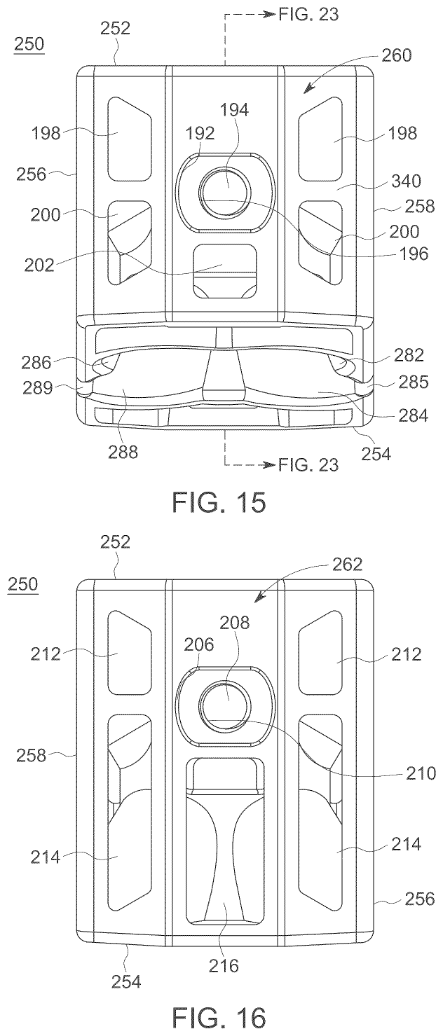

Referring now to FIG. 15, the frame 300 may include a first support member 340 on the fifth side 260 of the implant 250. The first support member 340 may include a first recessed region 192 extending into an intermediate portion from the fifth side 260. The first recessed region 192 may be as described in greater detail above with reference to implant 100. The first support member 340 may also include a first through hole 194 extending through the first support member 340. The first through hole 194 may extend, for example, entirely through the fifth side 260 of the implant 250. The first through hole 194 may include, for example, threads 196. The first support member 340 may also include at least one fifth window, superior window, or opening 198 extending through the first support member 340. The at least one fifth window 198 may include, for example, two parallel sides, a base portion connecting the two parallel sides at a first end, and a top portion connecting the two parallel sides at a second end. The base portion may be, for example, angled as it extends between the two sides. The at least one fifth window 198 may be, for example, at least two windows 198 positioned on a medial and lateral side of the fifth side 260. As shown in the depicted embodiment, the two fifth windows 198 may be positioned to be mirror images of each other and near a superior end the implant 250. The first support member 340 may also include at least one sixth window, inferior window, or opening 200 extending through the first support member 340. The at least one sixth window 200 may include, for example, two parallel sides, a base portion connecting the two parallel sides, and a top portion connecting the two parallel sides. The base portion may be, for example, angled as it extends between the two sides. The at least one sixth window 200 may be, for example, at least two windows 200 positioned on a medial and lateral side of the fifth side 260. As shown in the depicted embodiment, the two sixth windows 200 may be positioned to be mirror images of each other and near an inferior end of the fifth side 260 of the implant 250. The first support member 340 may also include at least one seventh window, intermediate window or opening 202. The at least one seventh window 202 may be positioned, for example, adjacent to the first recessed region 192. In the depicted embodiment, the at least one seventh window 202 is one seventh window 202 that is positioned inferior to the recessed region 192.

Referring now to FIG. 16, the frame 300 may include a second support member 342 on the sixth side 262 of the implant 250. The second support member 342 may include a second recessed region 206 extending into an intermediate portion from the sixth side 262. The second recessed region 206 may have, for example, a length, width and height and the length may be larger than the height. The second support member 342 may also include a second through hole 208 extending through the second support member 342. The second through hole 208 may extend, for example, entirely through the sixth side 262 of the implant 250. The second through hole 208 may include, for example, threads 210. The second support member 342 may also include at least one eighth window, superior window, or opening 212 extending through the second support member 342. The at least one eighth window 212 may include, for example, two parallel sides, a base portion connecting the two parallel sides at a first end, and a top portion connecting the two parallel sides at a second end. The base portion may be, for example, angled as it extends between the two sides. The at least one eighth window 212 may be, for example, at least two windows 212 positioned on a medial and lateral side of the sixth side 262. As shown in the depicted embodiment, the two eighth windows 212 may be positioned to be mirror images of each other and near a superior end the implant 250. The second support member 342 may also include at least one ninth window, inferior window, or opening 214 extending through the second support member 342. The at least one ninth window 214 may include, for example, two parallel sides, a base portion connecting the two parallel sides, and a top portion connecting the two parallel sides. The base portion may be, for example, angled as it extends between the two sides. The at least one ninth window 214 may be, for example, at least two windows 214 positioned on a medial and lateral side of the sixth side 262. As shown in the depicted embodiment, the two ninth windows 214 may be positioned to be mirror images of each other and near an inferior end of the sixth side 262 of the implant 250. The second support member 342 may also include at least one tenth window, intermediate window or opening 216. The at least one tenth window 216 may be positioned, for example, adjacent to the second recessed region 206. In the depicted embodiment, the at least one tenth window 216 is, for example, one tenth window 216 that is positioned inferior to the second recessed region 206.

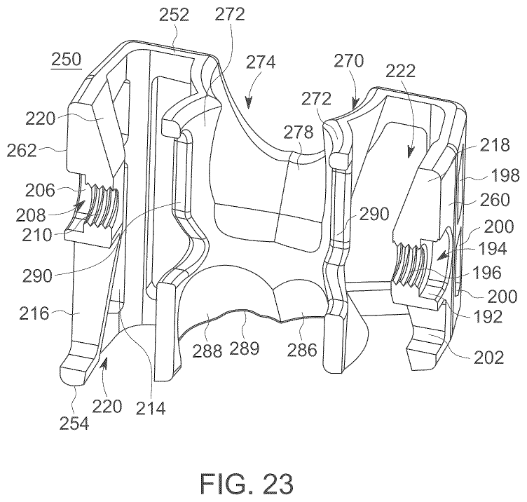

Referring now to FIGS. 14, 19, 20 and 23, the frame 300 may also include a first protrusion or interior protrusion 218 extending away from an interior surface of the fifth side 252. The first protrusion 218 may be positioned, for example, intermediate the third side 256 and the fourth side 258. The top surface of the first protrusion 218 may be, for example, angled as the top surface extends between the first side 252 and the second side 254, as shown in FIG. 23. The bottom surface of the first protrusion 218 may also be, for example, angled as the bottom surface extends between the second side 254 and the first side 252. The first through hole 194 may extend through the first protrusion 218.

Referring now to FIGS. 13, 19, 20 and 23, the frame 300 may include a second protrusion or interior protrusion 220 extending away from an interior surface of the sixth side 262. The second protrusion 220 may be positioned, for example, intermediate the third side 256 and the fourth side 258. The top surface of the second protrusion 220 may be, for example, angled as the top surface extends between the first side 252 and the second side 254, as shown in FIGS. 13 and 23. The bottom surface of the second protrusion 220 may also be, for example, angled as the bottom surface extends between the second side 254 and the first side 252. The second through hole 208 may extend through the second protrusion 220.

With continued reference to FIGS. 13, 14, and 19-23, the implant 250 may also include a first passageway 222 extending between the frame 300 and the inner body 270 near the first end 260. The first passageway 222 is open from the first side 252 to the second side 254. The first through hole 194 and the openings 198, 200, 202 extend from the exterior surface of the frame 300 through to the passageway 222. The implant 250 may further include a second passageway 224 extending between the frame 300 and the inner body 270 near the second end 254. The second passageway 224 is open from the first side 252 to the second side 254. The second through hole 208 and the openings 212, 214, 216 extend from the exterior surface of the frame 300 through to the passageway 224.

The implant 250 may also include a lattice structure 230, as shown in FIGS. 11 and 12. The lattice structure 230 may be, for example, positioned within the openings 316, 318, 334, 336, 198, 200, 202, 212, 214, 216, 222, 224 of the implant 250. The lattice structure 230 may be positioned, for example, within the first and second passageways 222, 224 to assist with bone fusion. As discussed in greater detail above with respect to FIG. 12 and which will not be described again here for brevity sake, the lattice structure 230 may include, for example, a plurality of recessed regions or grooves 232 between a plurality of raised portions 234 or may alternatively be, for example, printed within the passageways 222, 224. The lattice structure 230 may be, for example, offset from the outer frame of implant 250. The lattice structure 230 may be offset inside of the frame of the implant 250 by approximately 0.5 mm. Thus, the frame of the implant 250 is the only bone contacting element once implanted and the lattice structure 230 does not directly contact bone.

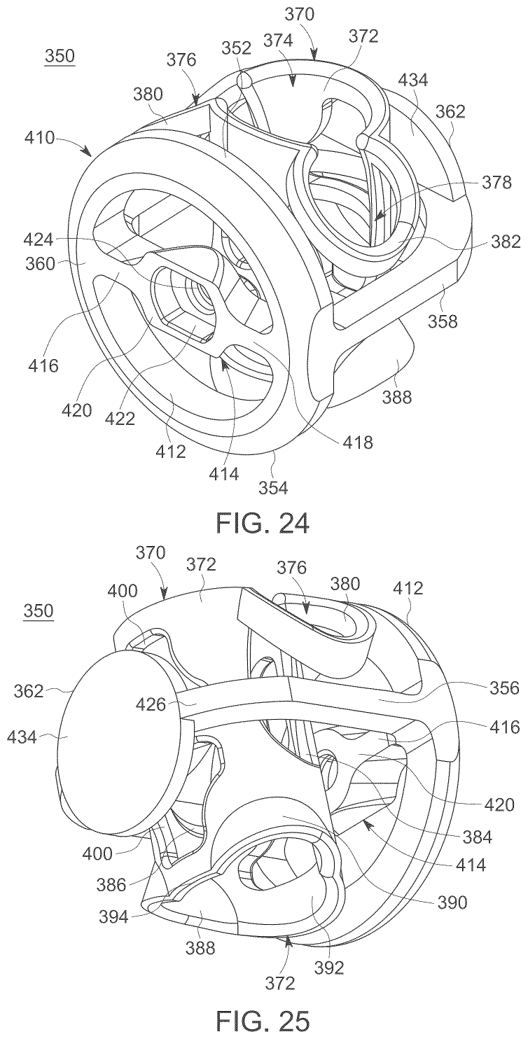

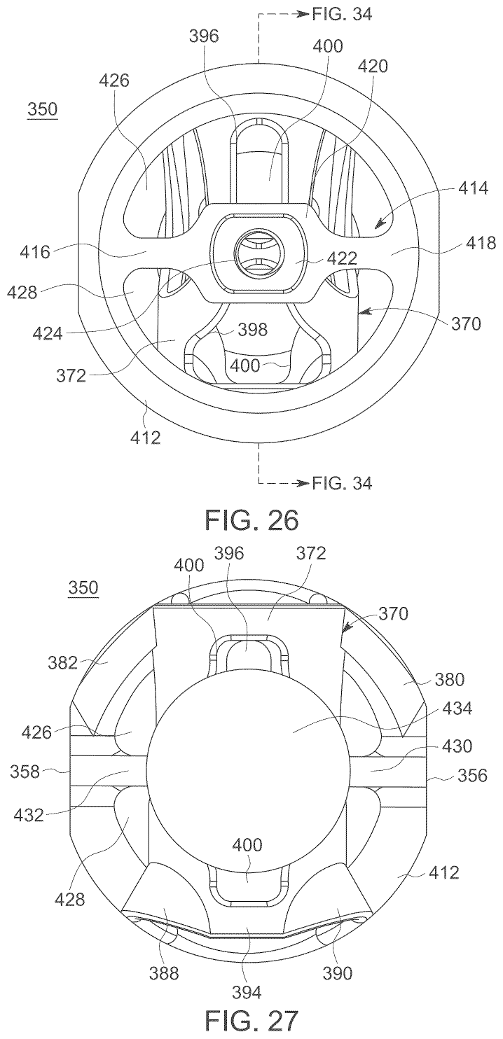

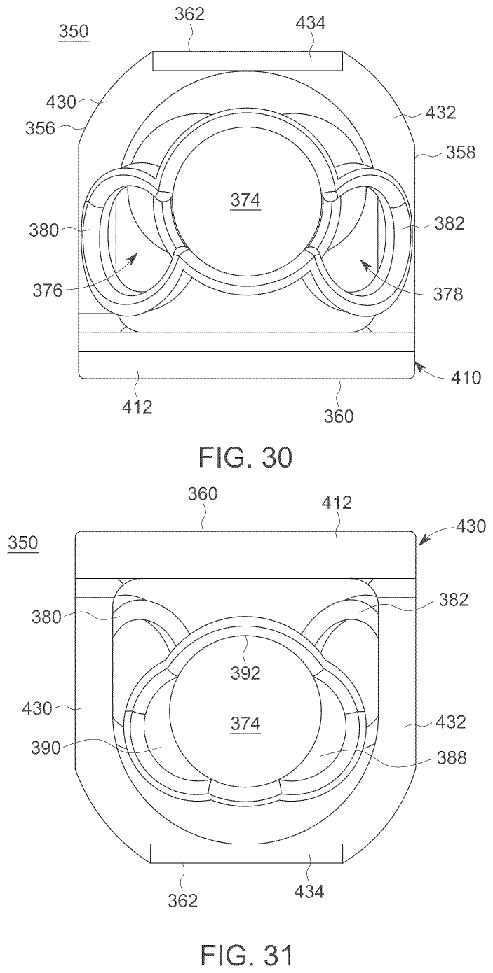

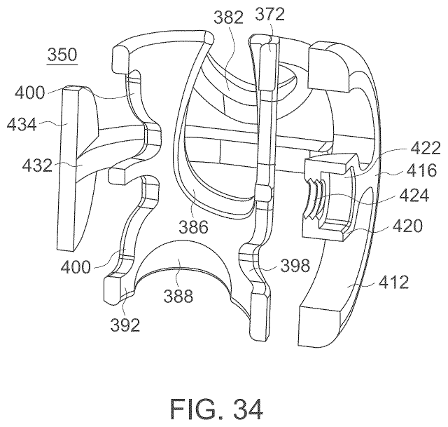

Referring now to FIGS. 24-34, another implant 350 is shown. The implant 350 may include a first side or top side 352, a second side or bottom side 354, a third side or right side 356, a fourth side or left side 358, a fifth side or first end 360, and a sixth side or second end 362. The first side 352 and the second side 354 may each be, for example, curved in opposite directions giving the implant 350 a generally cylindrical or bullet shape. The third side 356 may extend, for example, generally parallel to the fourth side 358. The fifth side 360 may extend, for example, generally parallel to the sixth side 362. The fifth side 360 and sixth side 362 may have, for example, round, circular or disk shapes and the diameter of the fifth side 360 may be larger than the diameter of the sixth side 362. The implant 350 may also include an inner body 370 surrounded by or positioned within a frame or housing 410.

The inner body 370 may include an intermediate portion 372 extending through the implant 350 from the first side 352 to the second side 354, as shown in FIGS. 24, 25 and 28-34. The intermediate portion 372 may form a through hole 374 extending through the inner body 370, as shown in FIGS. 24, 25, 30 and 31. The inner body 370 may also include a first guide hole or first access aperture 376 and a second guide hole or second access aperture 378. The first guide hole 376 may be, for example, angled in a first direction and the second guide hole 378 may be, for example, angled in a second direction. The first direction and the second direction may include trajectories extending in opposite directions. The trajectories of the first guide hole 376 and the second guide hole 378 may overlap as the trajectories extend through the implant 350 from a first side 352 to a second side 354.

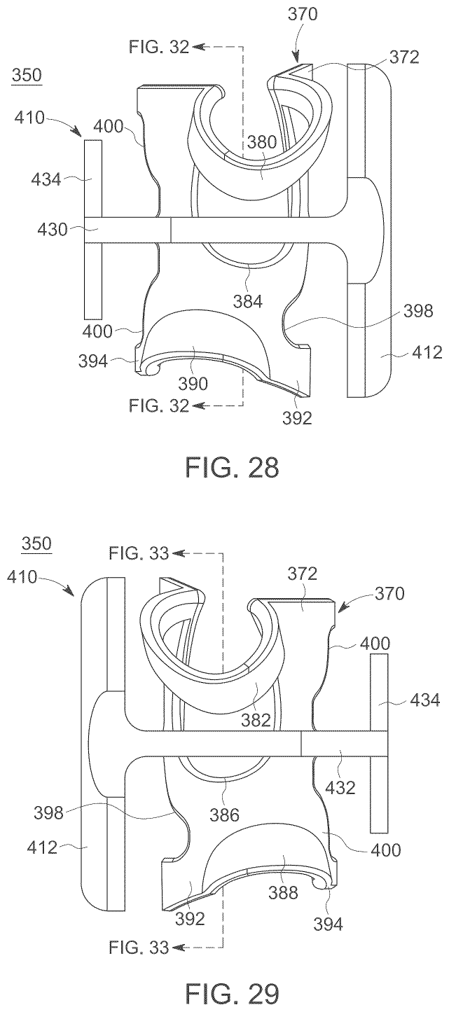

With continued reference to FIGS. 24-34, the first guide hole 376 may include a first support member 380 coupled to and extending away from the intermediate portion 372. The first support member 380 may be, for example, circular or round shaped. As shown in FIGS. 27-28, the first support member 380 may be, for example, curved as it extends away from the intermediate portion 372. The first guide hole 376 may also include a recessed region 384 and a first lower portion 388. The first lower portion 388 may, for example, extend away from the intermediate portion 372 and may have a semi-circular shape. The first lower portion 388 may be, for example, angled from the most proximal point of attachment to its free end. The first lower portion 388 may be, for example, positioned on a second side of the intermediate portion 372. The recessed region 384 may extend into the intermediate portion 372 on a first side. The recessed region 384 may, for example, extend down to a point allowing for a fastener (not shown) inserted in the opening formed by the first support member 380 to travel on the trajectory between the opening formed by the first support member 380 and through the recess formed by a first lower portion 388.

The second guide hole 378 may include a second support member 382 coupled to and extending away from the intermediate portion 372. The second support member 382 may be, for example, circular or round shaped. As shown in FIGS. 27 and 29, the second support member 382 may be, for example, curved as it extends away from the intermediate portion 372. The second guide hole 378 may also include a recessed region 386 and a second lower portion 390. The second lower portion 390 may, for example, extend away from the intermediate portion 372 and may have a semi-circular shape. The second lower portion 390 may be, for example, angled from the most proximal point of attachment to its free end. The second lower portion 390 may be, for example, positioned on a first side of the intermediate portion 372. The recessed region 386 may extend into the intermediate portion 372 on a second side of the intermediate portion 372. The recessed region 386 may, for example, extend down to a point allowing for a fastener (not shown) inserted in the opening formed by the second support member 382 to travel on the trajectory between the opening formed by the second support member 382 and through the recess formed by the second lower portion 390.

With continued reference to FIGS. 24-29, 32 and 33, the intermediate portion 372 of the inner body 370 may also include a first potion 392 and a second portion 394 extending between the first arm member 380 and the second lower portion 390 on one side and the second arm member 382 and first lower portion 388 on the other side. The intermediate portion 372 may also include at least one first channel or slot 396, 398 positioned on the first side 392 of the intermediate portion 372 and at least one second channel or slot 400 positioned on the second side 394 of the intermediate portion 372. The at least one first channel 396, 398 may be, for example, positioned opposite the at least one second channel 400. In addition, the first and second channels 396, 398, 400 may be positioned between the first support member 380 and the second support member 382.

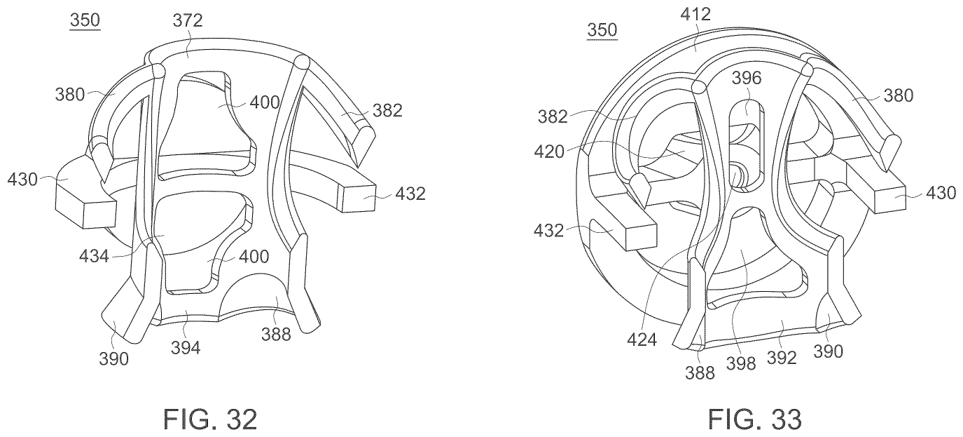

As shown in FIG. 33, the at least one first channel 396, 398 may include at least one first proximal channel 396 and at least one first distal channel 398. The first proximal channel 396 may be, for example, shaped as one of an oblong, oval, or a rectangle with rounded edges. The first distal channel 398 may be, for example, a generally triangular shape with straight or curved sides and pointed or flattened corners. As shown in FIG. 32, the second channels 400 may be, for example, shaped such that a first portion positioned near the top or bottom sides 352, 354 of the implant 350 has a smaller width than a second portion near a midpoint of the implant 350. The second channels 400 may include, for example, a first end opposite a second end and a first side and a second side extending from the free ends of the second side toward the first side. The second channels 400 may also include a third side extending between the first side at an end not coupled to the second end and a free end of the first end. The third side may be, for example, curved as the third side extends between the first side and the first end. The second channels 400 may further include a fourth side extending between the second side at an end not coupled to the second end and a free end of the first side opposite the third side. The fourth side may be, for example, curved as the fourth side extends between the second side and the first end.

With continued reference to FIGS. 24-34, the frame 410 may include a first base or first support 412 positioned at a first end of the frame 410 and a second base or fifth support 434 positioned at a second end of the frame 410. The first support 412 may be, for example, circular or oval shaped ring with an exterior surface, an interior surface, a first end or outside surface, and a second end or inside surface. The frame 410 may also include a second support or cross member 414 with a first end and a second end. The first and second ends of the second support 414 are coupled to an interior surface of the first base 412, for example, near a midpoint between the top side 352 and bottom side 354 of the implant 350. The second support 414 may include a first portion 416, a second portion 418, and a coupling portion 420. The first portion 416 is coupled to and extends from the interior surface of the first base 412 on the third side 356 of the implant 350 towards the midpoint and the second portion 418 is coupled to and extends from the interior surface of the first base 412 on the fourth side 358 towards the midpoint. The first portion 416 is coupled to the second portion 418 by a coupling portion 420 positioned, for example, at a midpoint of the first base 412. The coupling portion 420 may have, for example, a height in a direction between the top side 352 and the bottom side 354 larger than a height of each of the first portion 416 and the second portion 418. The first base 412 and second support 414 may have, for example, the same width in a direction between the first end 360 and the second end 362.

The coupling portion 420 may also include a recessed region 422 inset into the coupling portion 420 from the first end 360 towards an inside surface of the first base 412, as shown in FIGS. 24, 26 and 34. In addition, the coupling portion 420 may include a through hole 424 extending through the coupling portion 420 from the first end 360 toward an inside surface of the first base 412. The through hole 424 may be positioned, for example, centered in the recessed region 422. Thus, the through hole 424 may overlap with the recessed region 422 in the coupling portion 420. The first base 412 may also include a first opening 426 and a second opening 428 extending through the first base 412 from a first end 360 toward the second end 362. The first opening 426 may be positioned, for example, superior to the second portion 418 and the second opening 428 may be positioned, for example, inferior to the second portion 418. The first and second openings 426, 428 may be, for example, mirror images of each other.

The first base 412 is coupled to the second base 434 by a third support or first arm 430 and a fourth support or second arm 432. The first arm 430 may be positioned on a first side and the second arm 432 may be positioned on a second side opposite the first side. The first arm 430 may be positioned on, for example, the left side 356 of the implant 350 and the second arm 432 may be positioned on the right side 358 of the implant 350. The first arm 430 is positioned opposite to and parallel with the second arm 432. The second base 434 may have, for example, a round or circular cross-sectional shape. The first and second arms 430, 432 may be coupled to the second base 434 near a midline of the second base 434.

The implant 350 may also include a lattice structure 230, as shown in FIGS. 11 and 12. The lattice structure 230 may be, for example, positioned within the openings between the inner body 370 and the housing 410. The lattice structure 230 may be positioned, for example, within the space between the inner body 370 and the housing 410 to assist with bone fusion. As discussed in greater detail above with respect to FIG. 12 and which will not be described again here for brevity sake, the lattice structure 230 may include, for example, a plurality of recessed regions or grooves 232 between a plurality of raised portions 234 or may alternatively be, for example, printed within the openings between the inner body 370 and the housing 410. The lattice structure 230 may be, for example, offset from the outer frame of implant 350. The lattice structure 230 may be offset inside of the frame of the implant 350 by approximately 0.5 mm. Thus, the frame of the implant 350 is the only bone contacting element once implanted and the lattice structure 230 does not directly contact bone.

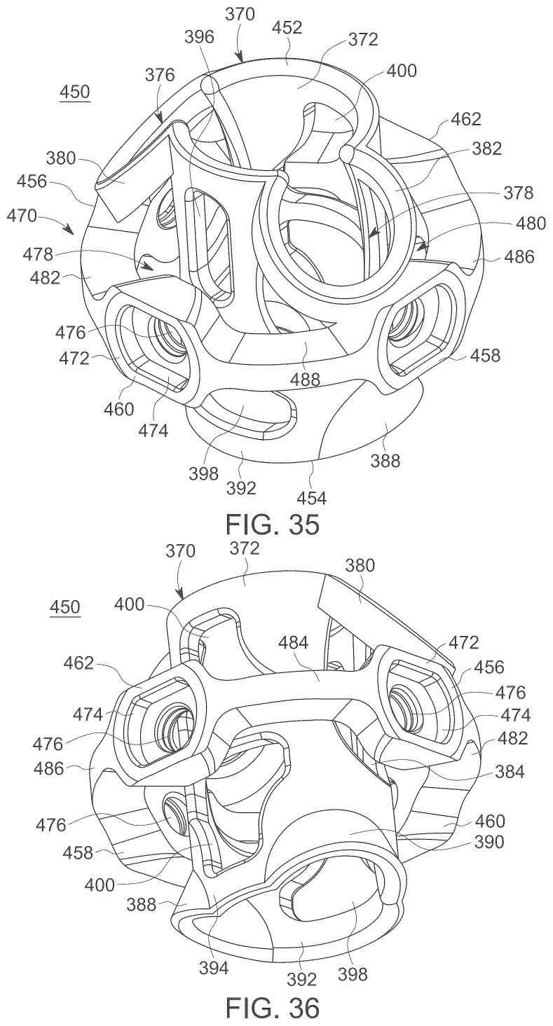

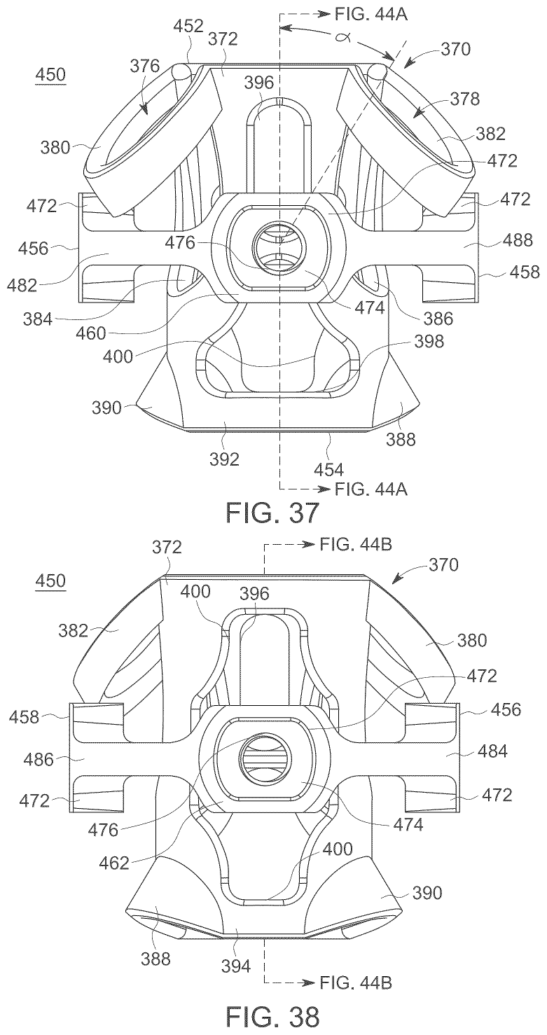

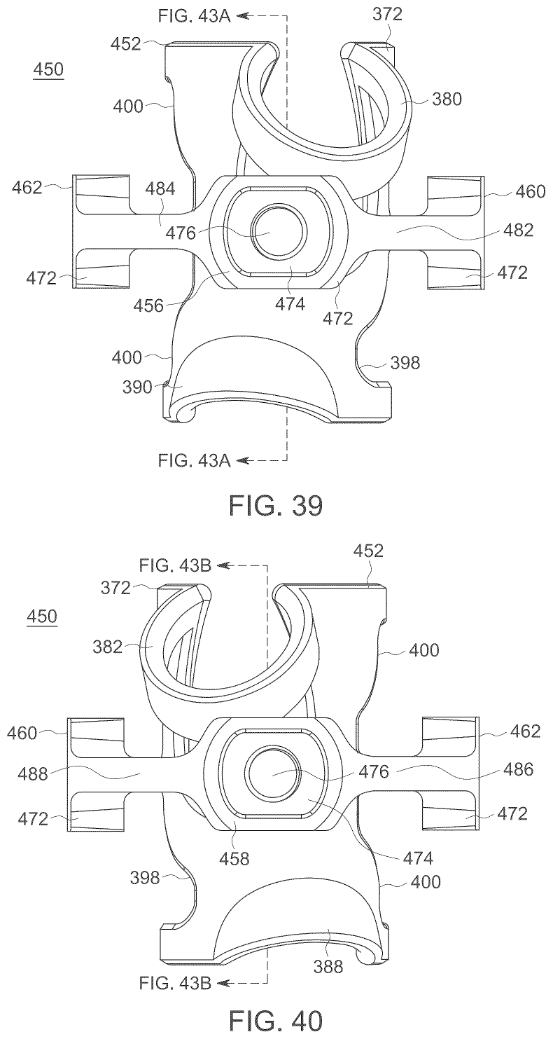

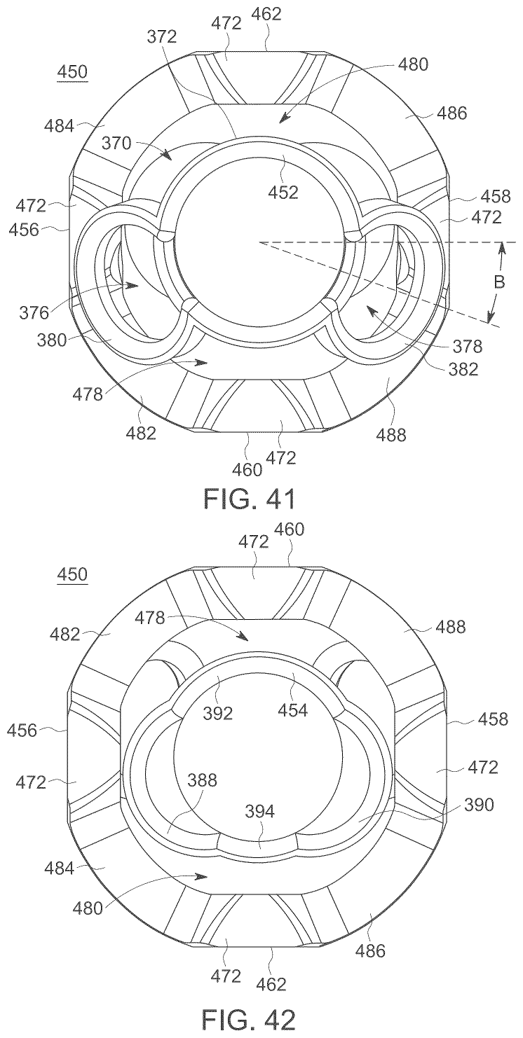

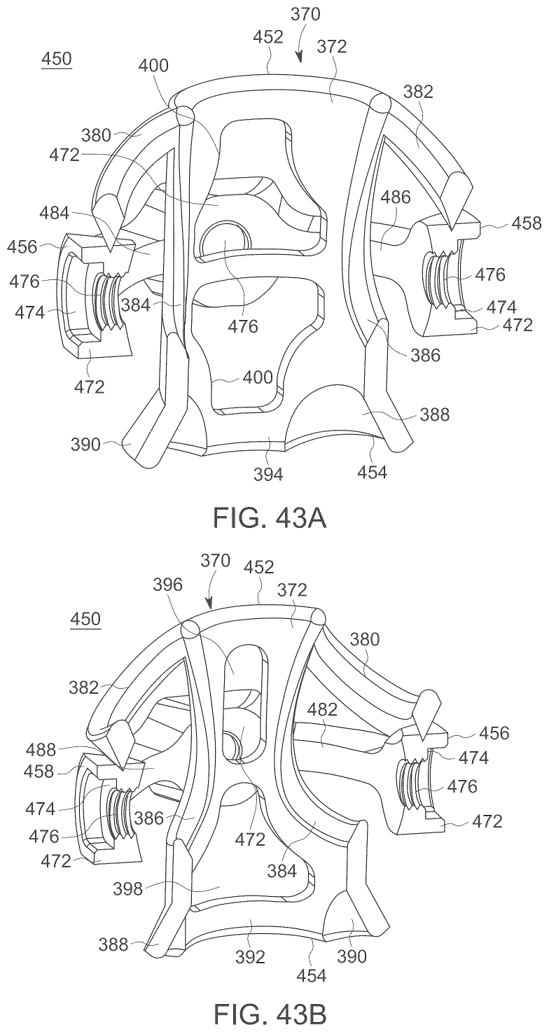

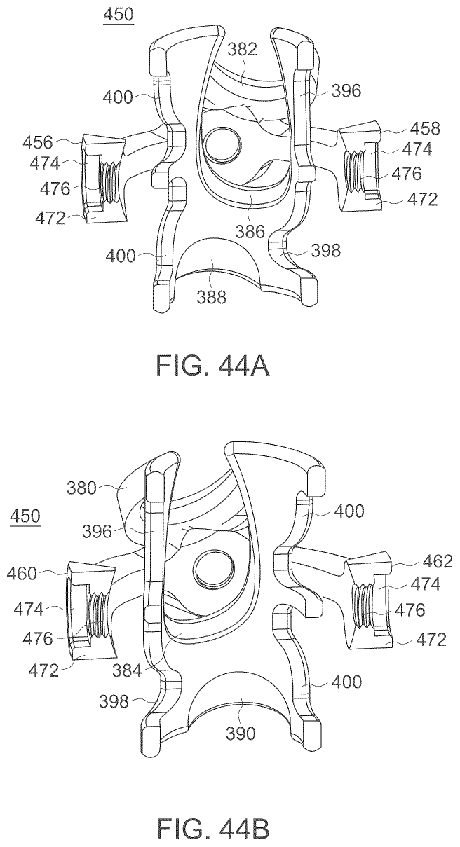

Referring now to FIGS. 35-45, another implant 450 is shown. The implant 450 may include a first side or top side 452, a second side or bottom side 454, a third side or right side 456, a fourth side or left side 458, a fifth side or first end 460, and a sixth side or second end 462. The first side 452 and the second side 454 may each be, for example, curved in opposite directions giving the implant 350 a generally round or ball shape. The third side 456 and the fourth side 458 may also each be, for example, curved in opposite directions. In addition, the fifth side 460 and the sixth side 462 may each be, for example, curved in opposite directions. The implant 450 may also include an inner body 370 surrounded by or positioned within a frame or housing 470. The inner body 370 may be as described in greater detail above with reference to implant 350, which will not be described again here for brevity's sake.

With continued reference to FIGS. 35-45, the housing 470 may include a plurality of coupling portions 472 and a plurality of support arms 482, 484, 486, 488 coupled to and connecting the coupling portions 472. The coupling portions 472 may be positioned, for example, on the third side 456, fourth side 458, fifth side 460, and sixth side 462. The coupling portion 472 positioned on the third side 456 is opposite the coupling portion 472 positioned on the fourth side 458. The coupling portion 472 positioned on the fifth side 460 is opposite the coupling portion 472 positioned on the sixth side 462. The first support arm 482 may extend between and couple the coupling portion 472 on the fifth side 460 to the coupling portion 472 on the third side 456. The second support arm 484 may extend between and couple the coupling portion 472 on the third side 456 to the coupling portion 472 on the sixth side 462. The third support arm 486 may extend between and couple the coupling portion 472 on the sixth side 462 to the coupling portion 472 on the fourth side 458. Finally, the fourth support arm 488 may extend between and couple the coupling portion 472 on the fourth side 458 to the coupling portion 472 on the fifth side 460.

The coupling portions 472 and arms 482, 484, 486, 488 of the housing 470 together form a ring around the inner body 370. The housing 470 may be, for example, coupled to at least a portion of the inner body 370. For example, the first arm member 380 may be coupled to a top of the coupling portion 472 positioned on the third side 456 of the implant 450. The second arm member 382 may be, for example, coupled to a top of the coupling portion 472 positioned on the fourth side 458 of the implant 450. The coupling portions 472 may have a first height extending in a direction between the first side 452 and the second side 454 and a first width extending from an exterior surface to an interior surface. The arms 482, 484, 486, 488 may have a second height extending in a direction between the first side 452 and the second side 454 and a second width extending from an exterior surface to an interior surface. The first height of the coupling portions 472 may be, for example, larger than the second height of the arms 482, 484, 486, 488. The first width of the coupling portion 472 may be, for example, the same or larger than the second width of the arms 482, 484, 486, 488.

Each of the coupling portions 472 may also include a recessed region 474 inset into the coupling portions 472 from the first end 460 towards an inside surface of the housing 470. In addition, the coupling portions 472 may include a through hole 476 extending through each coupling portion 472 from the first end 460 toward the inside surface of the housing 470. The through hole 476 may be positioned, for example, centered in the recessed region 474. Thus, the through hole 476 may overlap with the recessed region 474 in the coupling portions 472. The implant 450 may also include a first opening 478 extending between the inner body 370 and the housing 470 on a first side of the implant 450 and a second opening 480 extending between the inner body 370 and the housing 470 on a second side of the implant 450. The openings 478, 480 allow for insertion of graft material to assist with securing the implant 450 to the patient's ankle bones.

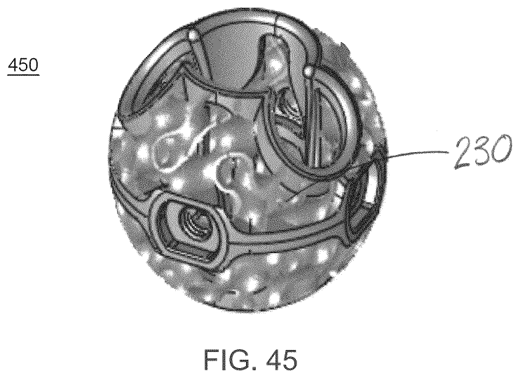

The implant 450 may also include a lattice structure 230, as shown in FIGS. 12 and 45. The lattice structure 230 may be, for example, positioned within the openings 478, 480 between the inner body 370 and the housing 470. The lattice structure 230 may be positioned, for example, within the space between the inner body 370 and the housing 470 to assist with bone fusion. As discussed in greater detail above with respect to FIG. 12 and which will not be described again here for brevity's sake, the lattice structure 230 may include, for example, a plurality of recessed regions or grooves 232 between a plurality of raised portions 234 or may alternatively be, for example, printed within the openings 478, 480 between the inner body 370 and the housing 470. The lattice structure 230 may be, for example, offset from the outer frame of implant 450. The lattice structure 230 may be offset inside of the frame of the implant 450 by approximately 0.5 mm. Thus, the frame of the implant 450 is the only bone contacting element once implanted and the lattice structure 230 does not directly contact bone.

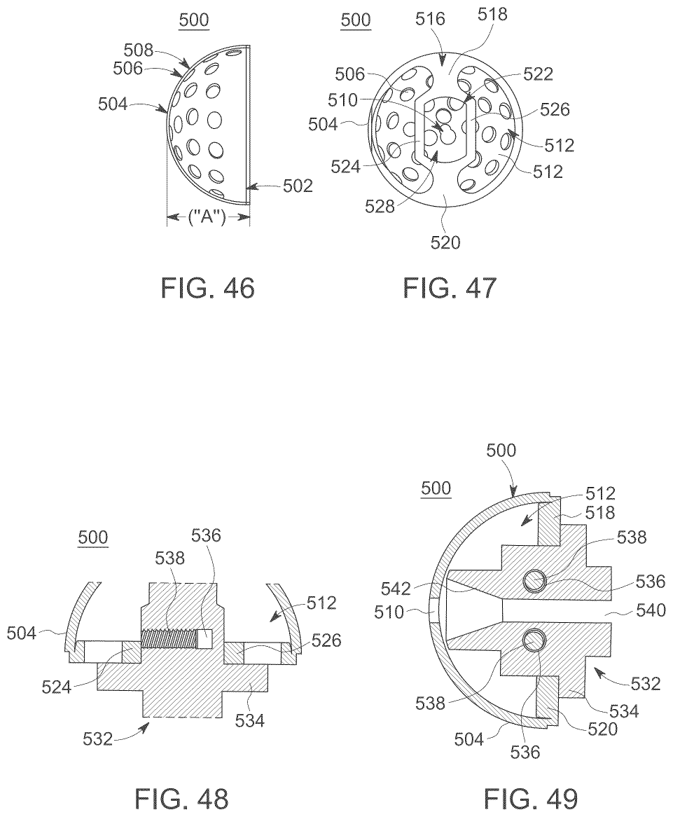

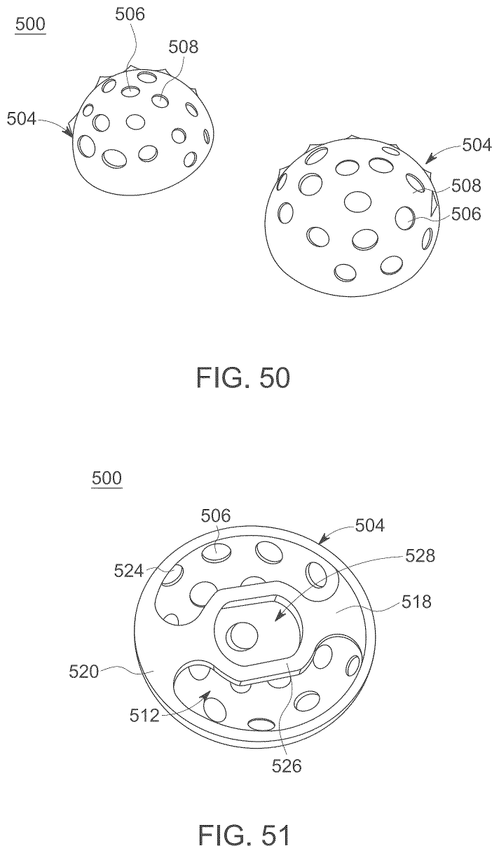

Referring now to FIGS. 46-51, a reaming instrument 500 is shown. The reaming instrument 500 may include a reaming head 502 and a reamer driver (not shown). The reamer driver may optionally include a tissue protector (not shown). The reaming head 502 may be attached to the reamer driver by ball detents, such as ball detents 530 shown in FIGS. 48-49. The reaming head 502 may have, for example, a hemispherical shape, a convex shape, or a shape corresponding to the shape of a spherical or cylindrical cage or implant, such as, implant 350, 450. The reaming head 502 may include an engagement surface or exterior surface 504 on an exterior of the reaming head 502 and an interior surface 512 opposite the engagement surface 504. The engagement surface 504 may include a plurality of openings 506 spaced over the engagement surface 504. In some embodiments, the plurality of openings 506 may be, for example, evenly or uniformly spaced over the entire engagement surface 504 and in other embodiments the openings 506 may be positioned over only a portion of the engagement surface 504. The openings 506 may extend from the exterior surface 504 through the reaming head 502 to the interior surface 512. Each of the openings 506 of the plurality of openings 506 may include an edge or portion 508, which may be, for example, a cutting edge or portion or a sharp edge or portion. The reaming head 502 may also include at least one guide wire opening or k-wire opening 510 extending through the reaming head 502 from the exterior surface 504 to the interior surface 512. The interior surface 512 may also have, for example, a hemispherical shape that corresponds to the shape of the engagement surface 504. Thus, the interior surface 512 may form a cavity or recess with, for example, a concave shape. The interior surface 512 may end at a rim 515 connecting the engagement surface 504 to the interior surface 512 at an end of the reaming head 502.

With continued reference to FIGS. 46-51, the reaming head 502 may also include an engagement member or coupling member 516 extending from one side of the reaming head 502 to another. The engagement member 516 may be, for example, coupled to a first portion of the interior surface 512 at the rim 515 on one side and a second portion of the interior surface 512 at the rim 515 on an opposite side to position the engagement member 516 extending across the cavity 514 of the interior surface 512. The engagement member 516 may include a first support arm 518 extending from a first side of the interior surface 512 toward a second side and a second support arm 520 extending from the second side of the interior surface 512 toward the first side. The first support arm 518 may be coupled to the second support arm 520 by a coupling member 522. The first and second support arms 518, 520 may have, for example, varying widths from the interior surface 512 of the reaming head 502 to the coupling member 522. For example, the sides of the arms 518, 520 may each have concave curvatures as the arms 518, 520 extend between the interior surface 512 and the coupling member 522. The coupling member 522 may include a first side 524, a second side 526, and a through hole 528 extending between the first side 524 and the second side 526. The first side 524 and the second side 526 both extend between and couple the first support arm 518 to the second support arm 520.

Referring now to FIGS. 48-49, the ball detents 530 include a housing 532 with a protrusion 534 extending away from a portion of the housing 532. The protrusion 534 engages the coupling member 522 of the reaming head 502. In addition, the housing 532 includes at least one opening 536 for receiving at least one spring 538. The at least one opening 536 is positioned to align the at least one opening 536 with at least one of the first side 524 and the second side 526 of the coupling member 522. The at least one spring 538 depresses when the ball detent 530 is inserted into the through hole 528 of the coupling member 522 and the exerts a force onto one of the first side 524 and the second side 526 to couple the reaming head 502 and the ball detent 530. The ball detent 530 also includes a cannulation 540 extending through the ball detent 530 from a first end to a second end. The ball detent 530 is inserted to align the cannulation 540 with the guide wire opening 510 in the reaming head 502. The cannulation 540 and opening 510 allow for a guide wire or k-wire to be inserted through the reaming instrument 500 and into the bone to be reamed. The ball detent 530 may also include a lead-in or tapered region 542 positioned at the second end. The tapered region 542 expands from the diameter of the majority of the cannulation 540 to a wider diameter at the second end of the ball detent 530.

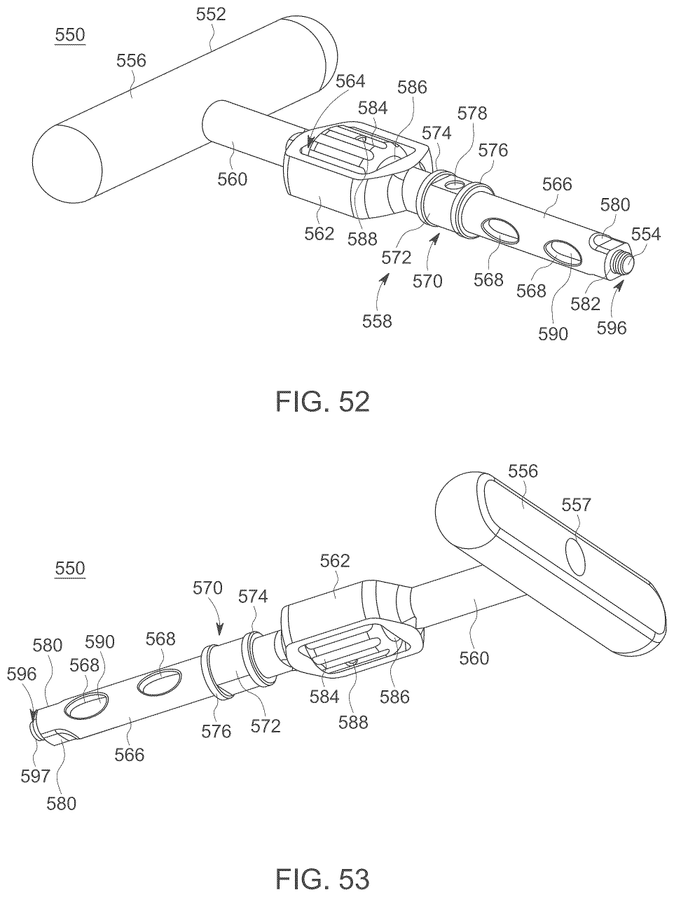

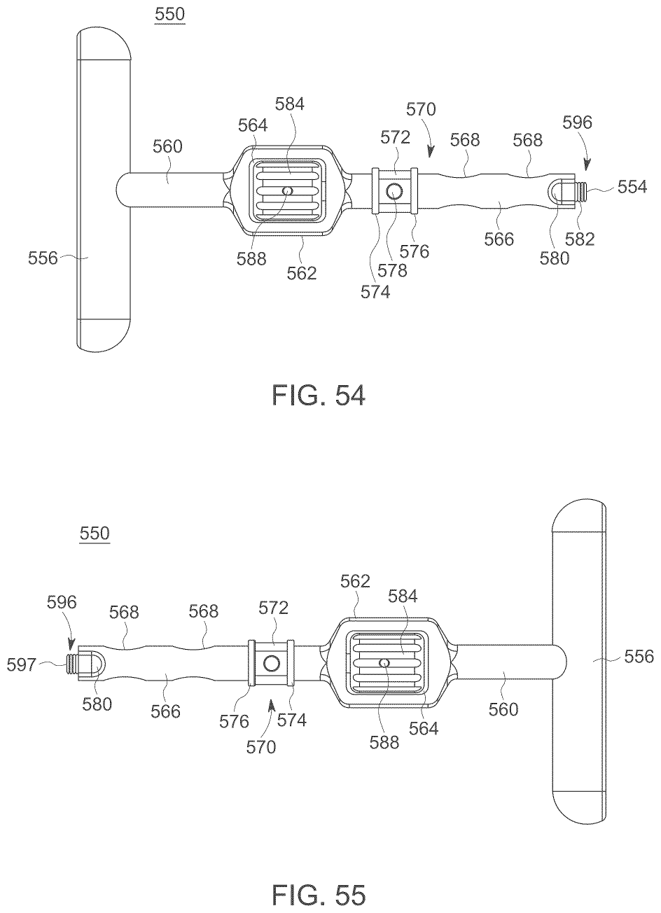

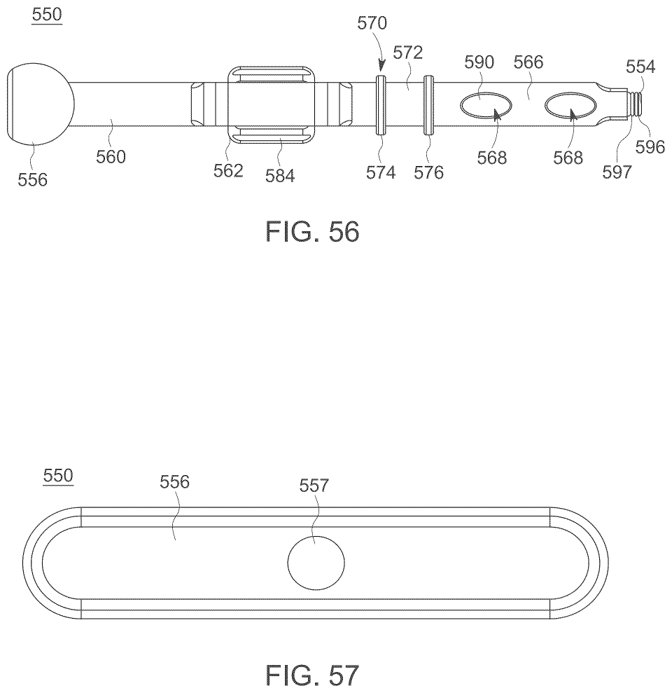

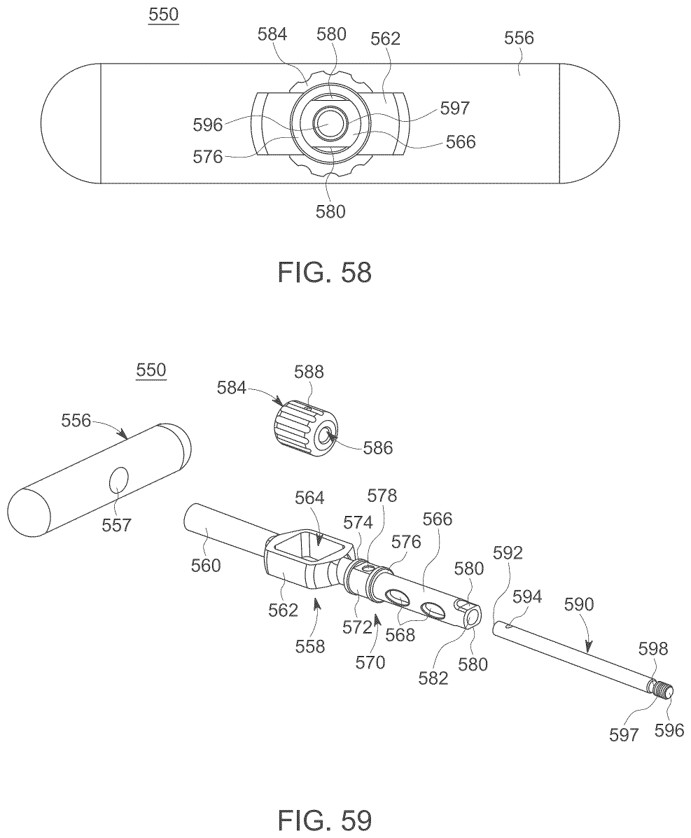

Referring now to FIGS. 52-60, an insertion instrument 550 is shown. The insertion instrument 550 includes a handle 556, a shaft 558 coupled to the handle 556, and an internal shaft 590 rotatably received within the shaft 558. The handle 556 is positioned at a first end 552 of the insertion instrument 550. The handle 556 may include an opening 557 extending through at least a portion of the handle 556 for receiving and coupling the shaft 558 to the handle 556. The shaft 558 may include a first portion 560, a knob housing 562, and a second portion 566. The first portion 560 is positioned at a first end of the shaft 558 and couples to and extends from the handle 556 toward the second end 554 of the insertion instrument 550. The knob housing 562 is coupled to the first portion 560 on a first end and the second portion 566 on a second end of the knob housing 562. The knob housing 562 includes an opening 564 extending through the knob housing 562 from a first side to a second side. The knob housing 562 includes a width and a height. The width and height of the knob housing 562 may be, for example, larger than the diameter of the first and second portions 560, 566 of the shaft 558. The opening 564 may be, for example, sized and shaped to receive a knob 584. The second portion 566 extends from the second end of the knob housing 562 to toward the second end 554 of the insertion instrument 550. The second portion 566 may include at least one through hole 568 extending through the second portion 566 in a direction perpendicular to a longitudinal axis of the shaft 558 extending between the first end 552 and the second end 554 of the insertion instrument 550. The at least one through hole 568 may be, for example, aligned with the longest portion of the handle 556. The at least one through hole 568 may be, for example, two through holes 568, although alternative numbers of through holes 568 are also contemplated. It is also contemplated that the through holes 568 may be positioned, for example, extending through the second portion 566 in any orientation.

With continued reference to FIGS. 52-60, the second portion 566 also includes a precision guide attachment portion 570. The attachment portion 570 may include an engagement surface or attachment surface 572 for engaging a precision guide, such as, precision guide 600. The attachment portion 570 may also include a first protrusion 574 on a first end of the engagement surface 572 and a second protrusion 576 on a second end of the engagement surface 572. The first and second protrusions 574, 576 may extend away from the second portion 566 of the shaft 558 circumferentially. The first and second protrusions 574, 576 assist with retaining the precision guide 600 in the desired position along the shaft 558 of the insertion instrument 550. The attachment portion 570 also includes a hole 578 extending into the engagement surface 572. The hole 578 may extend into one side of the engagement surface 572 through at least a portion of the second portion 566. The second portion 566 may also include at least one recessed region 580 on the second end of the shaft 558. The at least one recessed region 580 may be, for example, two recessed regions 580 positioned on opposite sides of the second portion 566. The at least one recessed region 580 may be, for example, tapered, curved or arched from a position offset from the second end 554 of the insertion instrument 550 and extending to the second end of the second portion 566.

In addition, the shaft 558 may include a passageway 582 extending from the second end of the second portion 566 of the shaft 558 to the opening 564 in the knob housing 562. The passageway 582 may be, for example, sized and shaped to receive the internal shaft 590. The knob 584 may be, for example, sized and shaped to be rotatably received within the opening 564 of the knob housing 562. The knob 584 may be, for example, cylindrically shaped and may include a plurality of grooves or other texture on the exterior surface to assist with rotating the knob 584. The knob 584 may include a through hole 586 extending through the knob 584 from a first end to a second end along a longitudinal axis. The knob 584 may also include a securement hole 588 extending through the knob 584 perpendicular to the through hole 586. The securement hole 588 may, for example, extend from the exterior textured surface through a first side of the knob 584 and into the through hole 586 and through at least a portion of a second side of the knob 584 opposite the hole 588 on the first side. The securement hole 588 may be, for example, sized and shaped to receive a pin or like coupling member to secure the internal shaft 590 to the knob 584.

As best seen in FIG. 59, the internal shaft 590 may include a first end 592 and a second end or engagement end 596. The first end 592 may include a through hole 594 for receiving a pin or like coupling member for securing the internal shaft 590 to the knob 584. The engagement end 596 may be, for example, threaded to engage and couple to the implants 100, 250, 350, 450 for insertion into a patient. The internal shaft 590 may also include a groove 598 positioned adjacent to the threads 597 of the engagement end 596. The internal shaft 590 may be inserted into the passageway 582 to position the through hole 594 within the knob 584 positioned within the knob housing 562. A pin may be inserted through a first side of the knob 584, the through hole 594 of the internal shaft 590, and at least a portion of the second side of the knob 584 to secure the internal shaft 590 to the knob 584. Once coupled together, turning of the knob 584 also turns the internal shaft 590. The engagement end 596 may extend at least partially out from the second portion 566 of the shaft 558. Specifically, the engagement end 596 may be positioned such that the threads 597 of the internal shaft 590 are extending out of the passageway 582 to engage an implant, such as implant 100, 250, 350, 450.

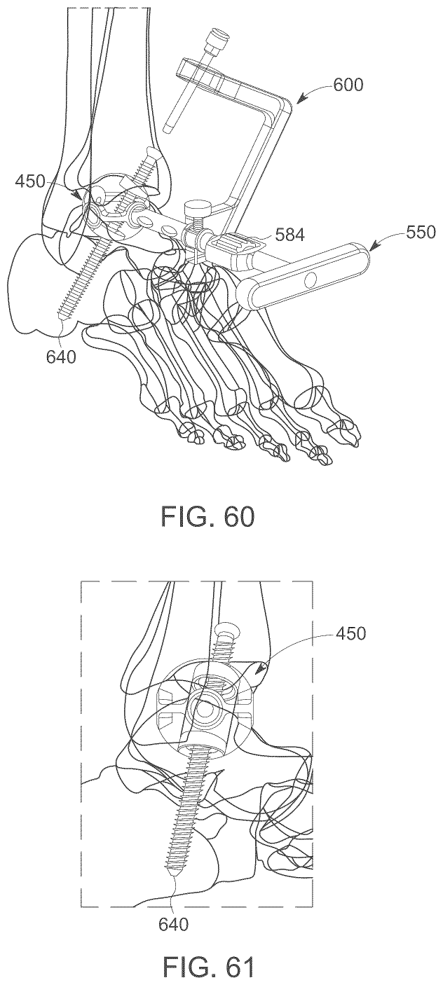

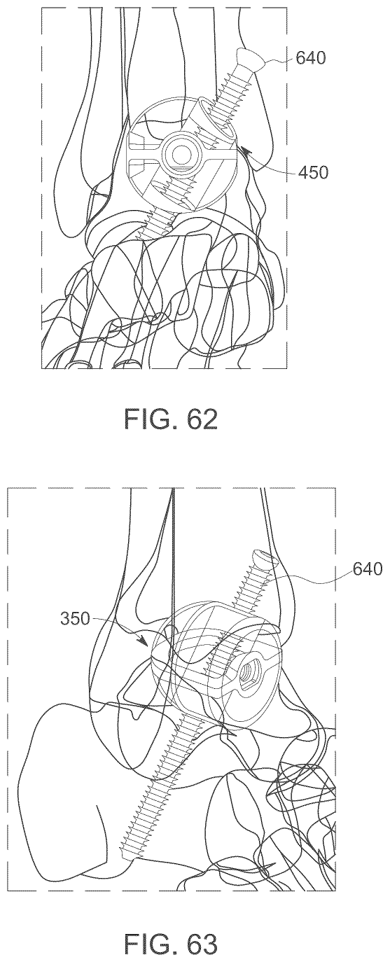

Referring now to FIGS. 60-63, implants 350, 450 are shown inserted into a patient's ankle. After the bones of the ankle are prepared for an implant, such as implants 350, 450, the implants 350, 450 may be coupled to an insertion instrument 550 and inserted into the patient's ankle. The engagement end 596 of the insertion instrument 550 may be, for example, inserted into the through hole 424, 476 of the implant 350, 450, respectively. A guide, such as guide 600, may then be coupled to the attachment portion 570 of the insertion instrument 550 for inserting a bone screw 640 through the implant 350, 450 and across the joint.

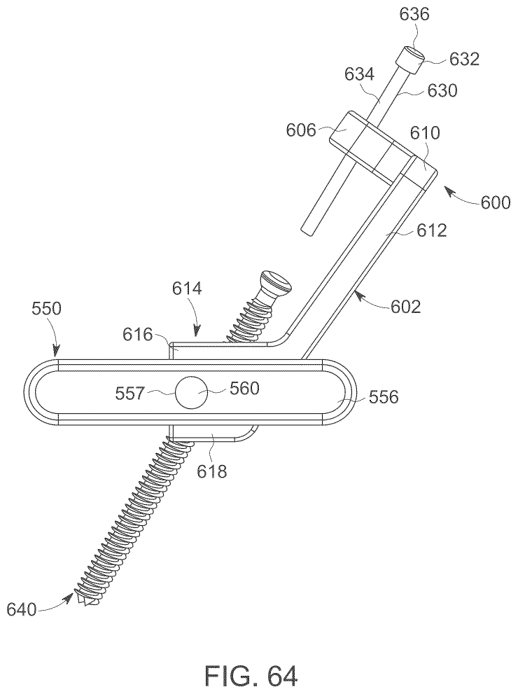

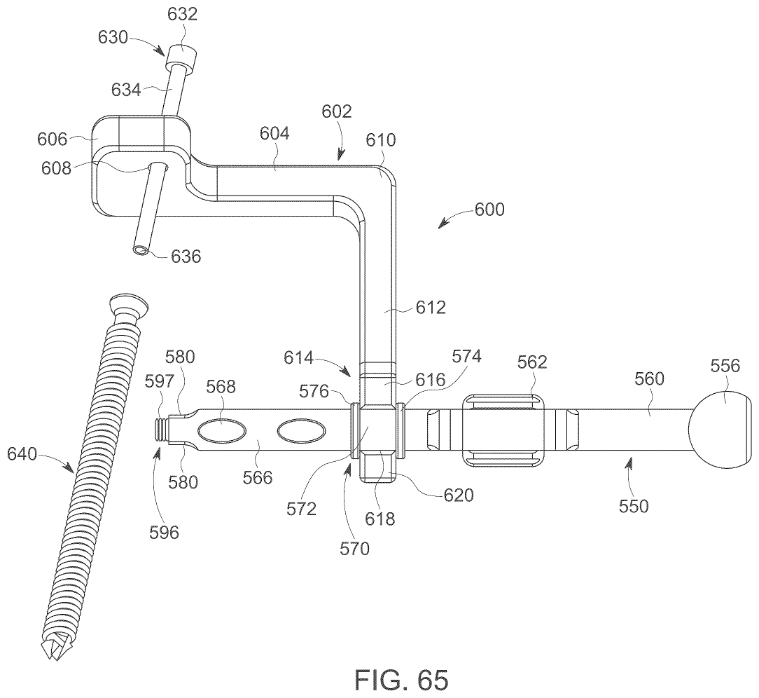

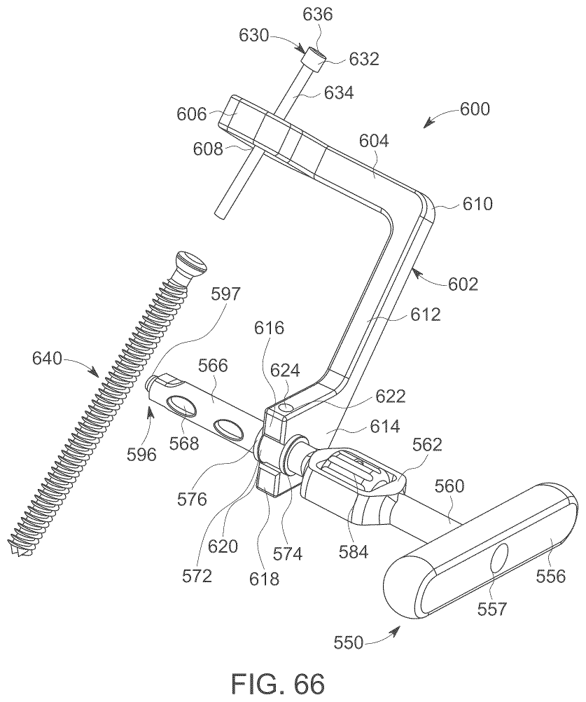

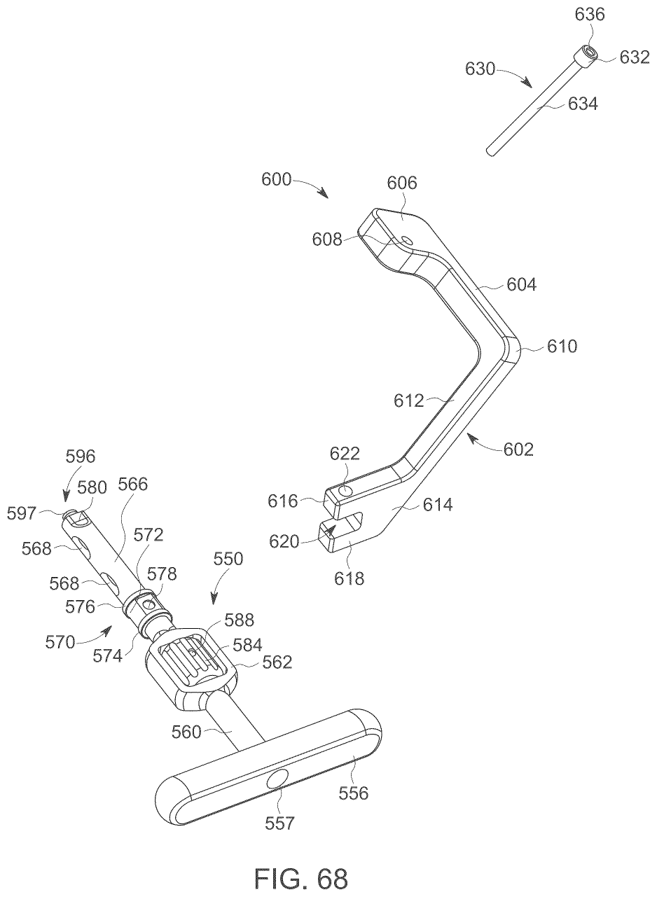

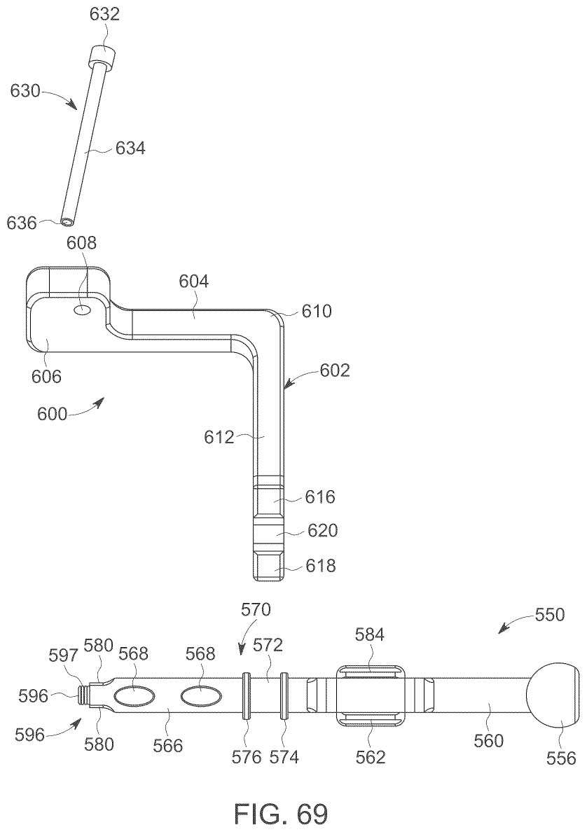

With continued reference to FIG. 60 and reference to FIGS. 64-69, the guide 600 is shown. The guide 600 may be, for example, a precision guide for cross screw placement, such as an anterior cross screw guide. The guide 600 may be, for example, an anterior cross screw guide. The guide 600 may include an arm 602 and a guide tube 630. The arm 602 may include, for example, a first portion 604 and a second portion 612 coupled to the first portion 604 at a coupling portion 610. The coupling portion 610 may be angled, for example, to position the first portion 604 at an angle relative to the second portion 612. The angle between the first portion 604 and the second portion 612 may be, for example, 75 degrees to 135 degrees, or more preferably 90 degrees to 120 degrees. The arm 602 may each have, for example, polygonal cross-sections, such as, generally square or rectangular cross-sections.

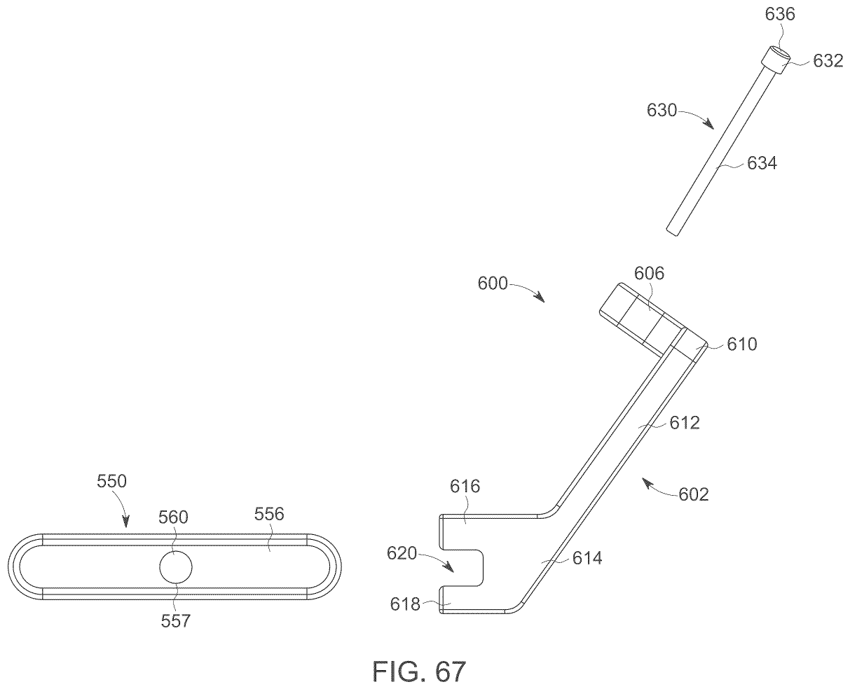

As best seen in FIGS. 67-69, the first portion 604 may include an alignment portion 606 at a first end of the first portion 604 and the coupling portion 610 at the second end of the first portion 604. The alignment portion 606 may extend from the first portion 604 generally perpendicular to a longitudinal axis of the first portion 604 extending between the coupling portion 610 and the first end. The alignment portion 606 may also include a through hole 608 extending through the alignment portion 606 from a top side to a bottom side. The alignment portion 606 may be positioned, for example, to align the through hole 608 with the through hole 123, 273, 374 of implant 100, 250, 350, 450, respectively, for insertion of the bone screw 640.

With continued reference to FIGS. 67-69, the second portion 612 may include an attachment portion 614 at a second end of the second portion 612 and the coupling portion 610 at the first end of the second portion 612. The attachment portion 614 may extend out from the second portion 612. The attachment portion 614 may include a first leg 616 positioned on a top side of the attachment portion 614 and a second leg 618 positioned on a bottom side of the attachment portion 614. The first leg 616 may be spaced from the second leg 618 forming a channel 620. The channel 620 may be, for example, sized and shaped to receive the engagement surface 572 of the attachment portion 570 of the insertion instrument 550. In addition, at least one of the first leg 616 and the second leg 618 may include a through hole 622 extending through the entire first leg 616 and/or second leg 618. In an embodiment, the through hole 622 may, for example, extend entirely through at least one of the first leg 616 and the second leg 618 and then partially through the other of the first leg 616 and the second leg 618. The through hole 622 may be aligned with the hole 578 of the insertion instrument 550 and a securement pin 624 may be inserted through the hole 622 and the hole 578 to couple the precision guide 600 to the insertion instrument 550. Once the precision guide 600 is coupled to the insertion instrument 550, any rotation and/or translation of the implant 100, 250, 350, 450 is immediately applied to the position of the guide 600 and adjusts the cross screw trajectory to match the adjustment of the implant 100, 250, 350, 450.

With continued reference to FIGS. 64-69, the guide tube 630 may include a head 632 at a first end of the guide tube 630 and a shaft 634 extending from the head 632 to the second end of the guide tube 630. The guide tube 630 may also include a cannulation or through hole 636 extending through the guide tube 630 from the first end to the second end along the longitudinal axis of the guide tube 630. The guide tube 630 may be, for example, a tissue protector, a k-wire guide, a drill guide, etc. as known by one of ordinary skill in the art to assist with the insertion of a bone screw 640 using the precision guide 600.

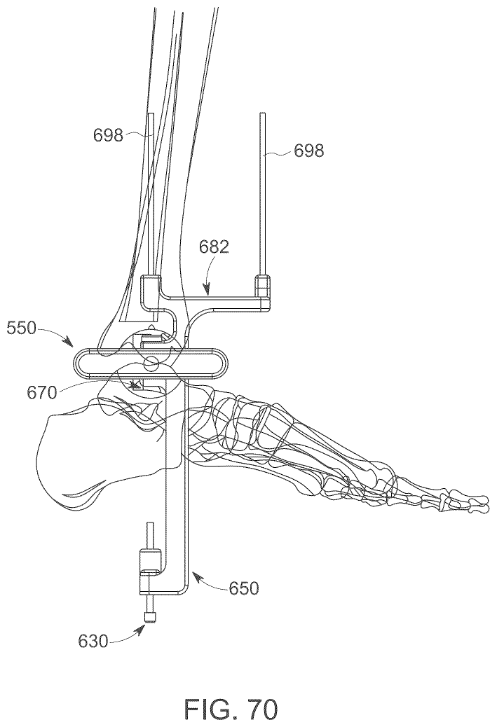

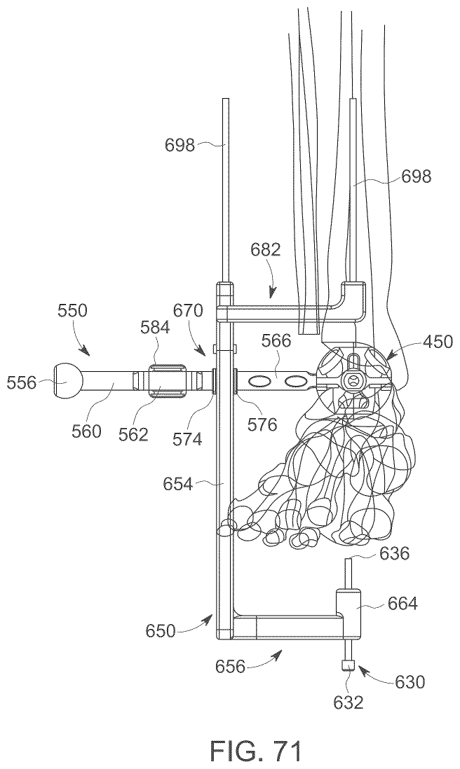

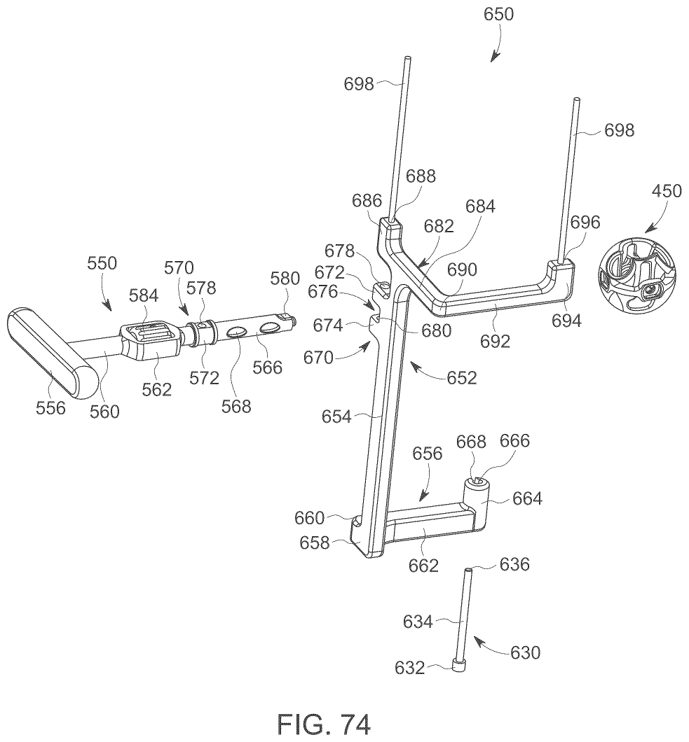

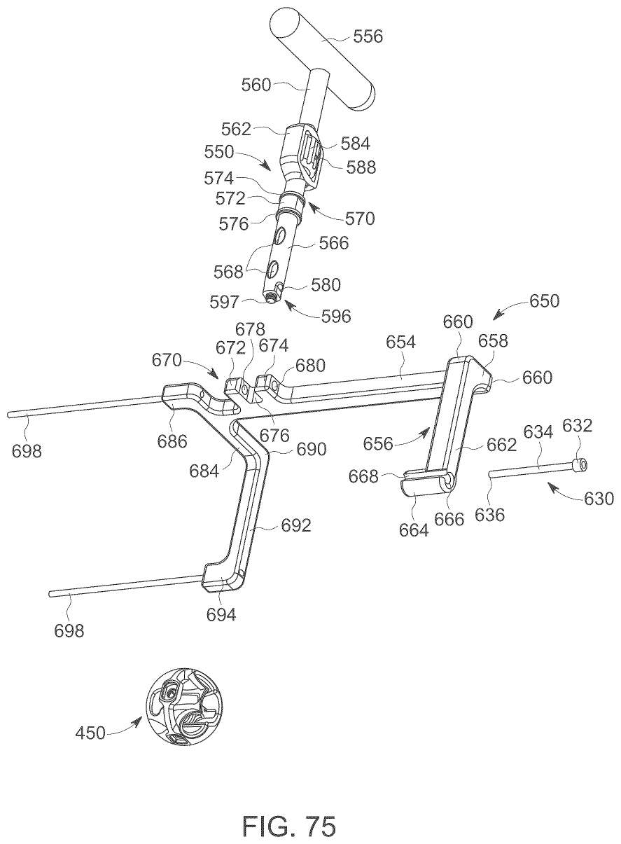

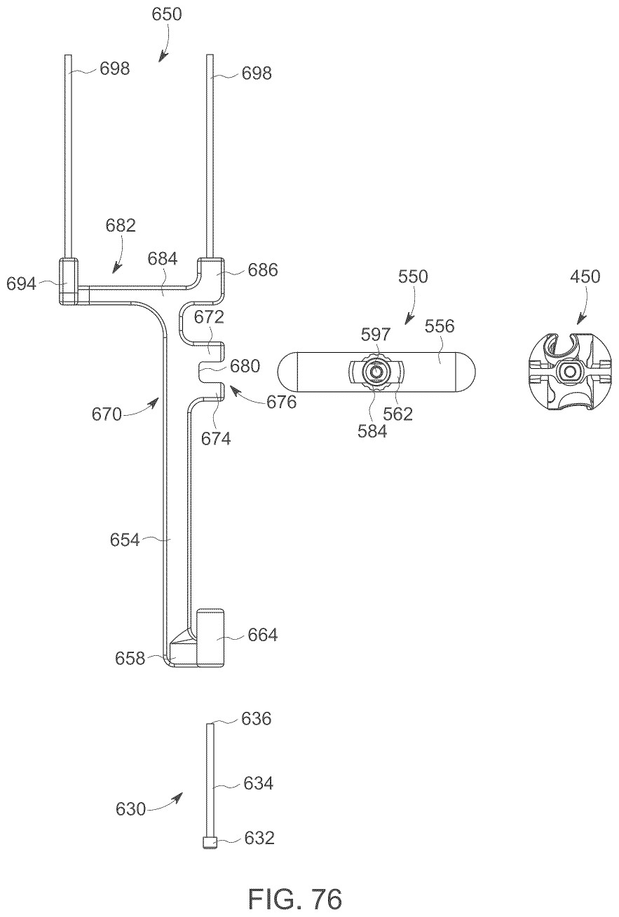

Referring now to FIGS. 70-76, another insertion system 650 is shown. The insertion system 650 includes a precision guide for placement of an intramedullary fastener, such as a lateral nail guide. The guide 650 may be, for example, a lateral cross screw guide. The guide 650 may include a frame 652, a guide tube 630, and at least one angle wing 698. The frame 652 may include a base member 654, a foot 656 at one end of the base member 654, and an arm 682 at another end of the base member 654. The base member 654, foot 656, and arm 682 may each have, for example, polygonal cross-sections, such as, generally square or rectangular cross-sections.

As shown in FIGS. 73-76, the foot 656 includes a first foot portion 658 extending in a first direction from the base member 654. The first foot portion 658 extends to a foot connector 660 which couples the first foot portion 658 to a second foot portion 662. The connector 660 is angled, for example, at 90 degrees or at approximately 90 degrees to position the second foot portion 662 extending in a second direction. The second foot portion 662 is coupled to the foot connector 660 at a first end and includes a sleeve 664 at a second end. The sleeve 664 extends from a bottom side of the second end of the second foot portion 662 past a top side of the second foot portion 662 and away from the top side of the second foot portion 662. The sleeve 664 may have, for example, a cylindrical shape. The sleeve 664 may also have a through hole 666 extending from a top side to a bottom side of the sleeve 664. The through hole 666 may be, for example, sized and shaped to receive a guide tube 630, as described in greater detail above and which will not be described again here for brevity sake. In addition, the sleeve 664 may include a channel 668 extending from an exterior surface of the sleeve 664 into the through hole 666. The channel 668 may be, for example, spaced apart from the point of attachment of the sleeve 664 to the second foot portion 662. The channel 668 may extend from the top side to the bottom side of the sleeve 664. The channel 668 may have, for example, a width that is smaller than the diameter of the guide tube 630.

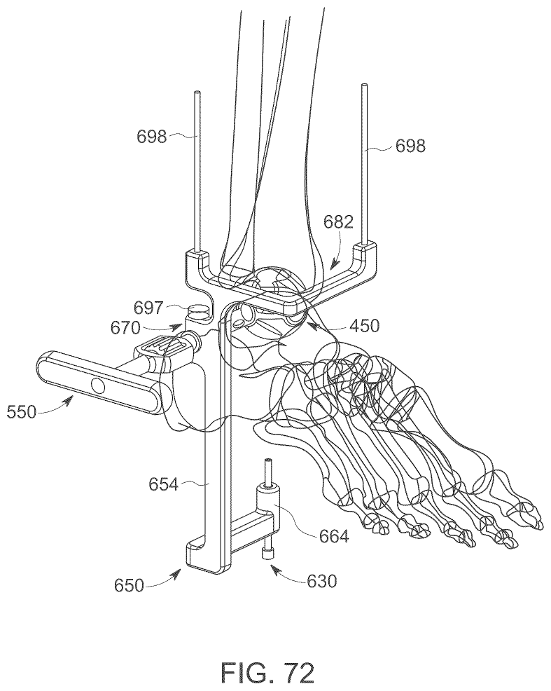

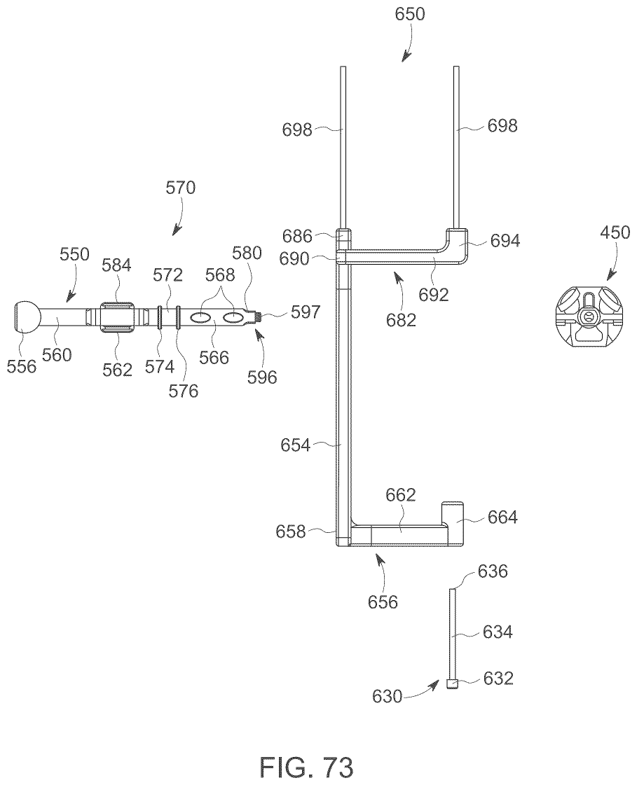

With continued reference to FIGS. 73-76, the base member 654 may also include an attachment portion 670 for coupling to the attachment portion 570 of the insertion instrument 550. The attachment portion 670 may include a first leg 672 and a second leg 674. The legs 672, 674 each extend away from the base member 654 in the first direction. The first leg 672 may be, for example, spaced apart from the second leg 674 along the length of the base member 654 forming a channel 676 extending between the legs 672, 674 to the base member 654. The channel 676 may be, for example, sized and shaped to receive the engagement surface 572 of the attachment portion 570. The first leg 672 may include a first through hole 678 extending through the free end of the first leg 672 from a top surface to a bottom surface. The second leg 674 may include a second through hole 680 extending through the free end of the second leg 674 from a top surface to a bottom surface. The through holes 678, 680 may be aligned with the hole 578 of the insertion instrument 550 and a securement pin 697 may be inserted through the first hole 678, the hole 578, and the second hole 680 to couple the precision guide 650 to the insertion instrument 550, as shown in FIG. 72. Once the precision guide 650 is coupled to the insertion instrument 550, any rotation and/or translation of the implant 100, 250, 350, 450 is immediately applied to the position of the guide 650 and adjusts the cross screw trajectory to match the adjustment of the implant 100, 250, 350, 450.

In addition, as shown in FIGS. 73-76, the arm 682 may be coupled to the base member 654 at a first end of the base member 654. The arm 682 may include a first arm portion 684 and a second arm portion 692 coupled to the first arm portion 684 by an arm connector 690. The base member 654 may be coupled to the first arm portion 684 between a first end and a second end of the first arm portion 684. The first arm portion 684 may extend away from the base member 654 in both a first direction and a third direction opposite the first direction. The first end of the first arm portion 684 may include a first alignment portion 686 extending away from a top surface of the first arm portion 684. The first alignment portion 686 may include a first opening 688 extending into the first alignment portion 686 toward the first arm portion 684. The first opening 688 may be, for example, sized and shaped to receive an angle wing 698. The arm connector 690 may be, for example, positioned to change the direction of the second arm portion 692 with respect to the first arm portion 684 with the first arm portion 684 extending in the first and third direction and the second arm portion 692 extending in the second direction. The connector 690 may also be angled, for example, to position the first arm portion 684 at an angle relative to the second arm portion 692. The angle between the first arm portion 684 and the second arm portion 692 may be, for example, 80 degrees to 110 degrees, or more preferably about 90 degrees.

With continued reference to FIGS. 73-76, the second arm portion 692 is coupled to the arm connector 690 at a first end and a second alignment portion 694 at a second end. The second end of the second arm portion 692 may include a second alignment portion 694 extending away from a top surface of the second arm portion 692. The second alignment portion 694 may include a second opening 696 extending into the second alignment portion 694 toward the second arm portion 692. The second opening 696 may be, for example, sized and shaped to receive an angle wing 698. The two angle wings 698 may assist with alignment of the frame 652 with respect to a patient's bones. The arm 682 may each have, for example, polygonal cross-sections, such as, generally square or rectangular cross-sections.

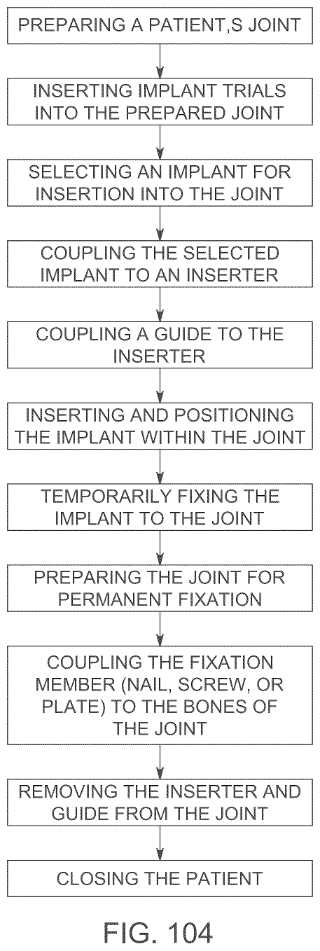

As shown in FIG. 104, the surgical method for an ankle fusion procedure for the insertion of one of implants 100, 250, 350, 450 includes preparing the joint. For implants 100, 250, the joint may be prepared by, for example, using a combination of oscillating and reciprocating saw blades (not shown) with cut guides to create a cavity or pocket for receiving the selected implant 100, 250. The cut guides may be, for example, sized and shaped to allow for the joint to be cut to the shape of the implants 100, 250. For implants 350, 450, the joint may be prepared using a reaming instrument, such as reaming instrument 500 which includes hemispherical reaming heads 502. The reaming instrument 500 may be used free hand or over a k-wire to shape the existing bone void to create a cavity or pocket sized to receive the implant 350, 450. The autograft bone chips created using the reaming instrument 500 may be collected from the reaming instrument 500 and saved for later use in graft packing. It is also contemplated that the reaming instrument 500 may be used with, for example, a parallel reaming guide to ensure that the reaming path is formed perpendicular to the long axis of the tibia. The parallel reaming guide may be configured, for example, to directly or indirectly engage the reaming instrument 500 and be used to ensure the reamer is inserted to form a reaming path perpendicular to the long axis of the tibia. Ensuring the reaming path is perpendicular allows for the intramedullary nail to be correctly aligned and placed through the implant 350, 450. The parallel reaming guide may include openings to allow for the guide to be fixed to the tibia in multiple locations. Optionally, fluoroscopy may be used to assist with reaching the correct depth in the bone with the reaming instrument 500, as well as ensuring that the parallel reaming guide is positioned perpendicular to the tibia.

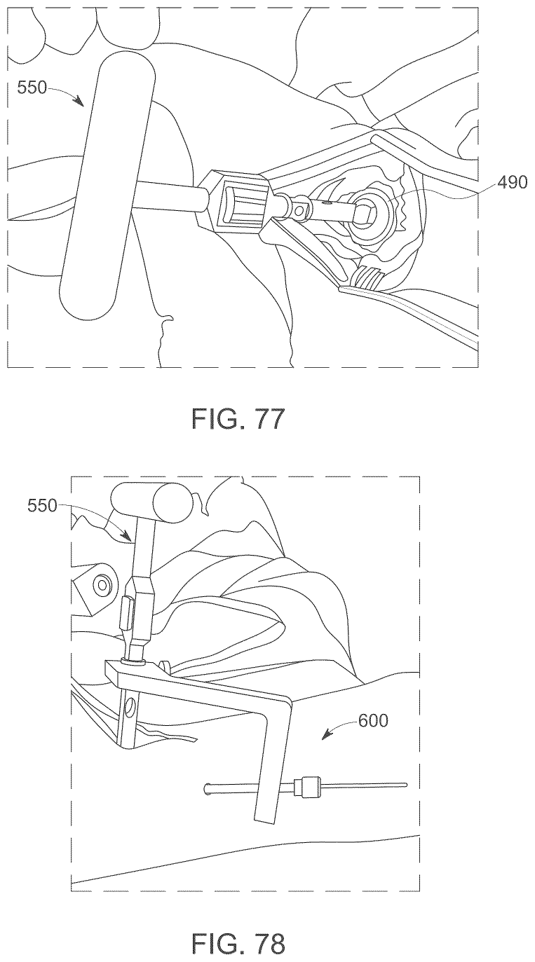

The method then includes trialing the implants 100, 250, 350, 450 to determine the correct sizing for the prepared joint. The implant trials 490 may be attached to the insertion instrument 550 and inserted into the reaming path to determine the size and shape implant 100, 250, 350, 450 that fits best within the cavity or pocket within the joint, as shown in FIG. 77. Once a specific sized and shaped implant 100, 250, 350, 450 is selected it may be removed from the insertion instrument 550 and the selected implant 100, 250, 350, 450 may be coupled to the inserter 550. The selected implant 100, 250, 350, 450 is coupled to the inserter 550 by inserting and threadingly engaging the threaded end 596 of the insertion instrument 550 into the first through hole 194, 424, 476 of the implants 100, 250, 350, 450 and inserting the recessed regions 580 of the shaft 558 of the insertion instrument 550 into the recessed region 192, 422, 474 of the selected implant 100, 250, 350, 450, as shown in FIG. 77. By inserting the recessed regions 580 of the shaft 558 into the recessed region 192, 422, 272 of the selected implant 100, 250, 350, 450, the surgeon may manipulate the implant 100, 250, 350, 450 in rotation, as well as translation in all planes. The threaded end 596 of the inserter 550 is threadingly coupled to the through hole 194, 424, 476 of the implant 100, 250, 350, 450 by rotating the knob 584 of the inserter 550 until the threads 597 of the inserter 550 engage the threads of the through hole 194, 424, 476.

Next, the method includes coupling a precision guide 600, 650 to the inserter 550 for fixation. Fixation can involve, for example, an intramedullary nail or a cross screw and plate. The plate may be, for example, a plate such as those described in U.S. Provisional Application No. 63/227,138 filed Jul. 29, 2021 and entitled SPAN BONE PLATES, which application is hereby incorporated by reference in its entirety. As discussed in greater detail above and which will not be described again here for brevity sake, the attachment portion 614, 670 of the precision guides 600, 650 may be coupled to the precision guide attachment portion 570 of the inserter 550 for insertion of a cross screw or intramedullary nail across the joint and through the implant 100, 250, 350, 450. Once the guide 600, 650 is coupled to the inserter 550 any translation or rotation of the inserter 550 and coupled implant 100, 250, 350, 450 will immediately be applied to the position of the guide 600, 650 without any further action by the surgeon. The implants 100, 250, 350, 450 may all be translated. In addition, the implant 450 may be rotated in all planes and the implant 350 may be rotated in one plane. Thus, as the inserter 550 and coupled implant 100, 250, 350, 450 are moved, the trajectory of the cross screw or intramedullary nail from the guide 600, 650 is maintained.

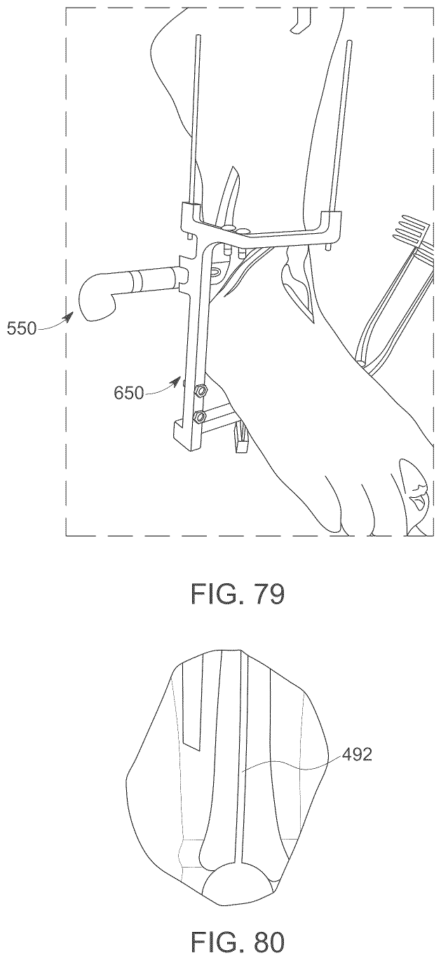

In one embodiment, once the desired position of the implant 100, 250, 350, 450 is achieved, a k-wire may be inserted through the guide tube 630 of the guide 600 and across the joint, as shown in FIG. 78. After placement of the k-wire, the guide 600 may be removed and a drill inserted over the k-wire and through the joint. After the opening is drilled, a cross screw may be inserted into the opening and across the joint. In another embodiment, once the desire position of the implant 100, 250, 350, 450 is achieve, the central nail hole in the implant 100, 250, 350, 450 may be aligned with the long axis of the tibia using the angle wings 698 and fluoroscopy in both the anterior-posterior and medial-lateral views. Once aligned, the guide 650 may be temporarily fixed to the bones, as shown in FIG. 79. Then, a k-wire 492 may be inserted through the guide tube 630 of the guide 650 and up into the tibial canal, as shown in FIG. 80. After placement of the k-wire, the guide 650 may be removed and a drill inserted over the k-wire and through the joint. After the opening is drilled, an intramedullary nail may be inserted into the opening and across the joint.

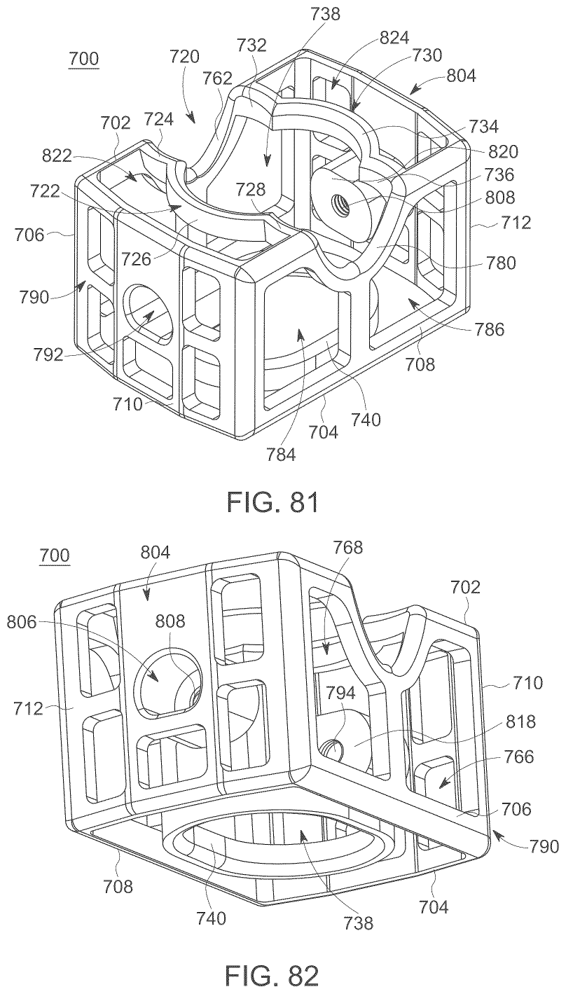

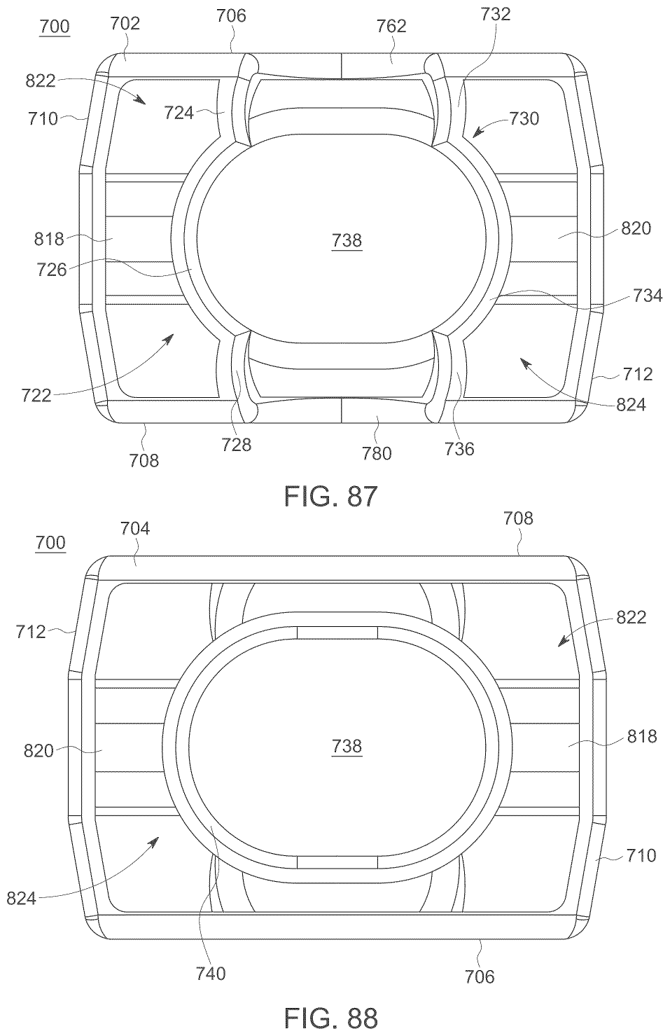

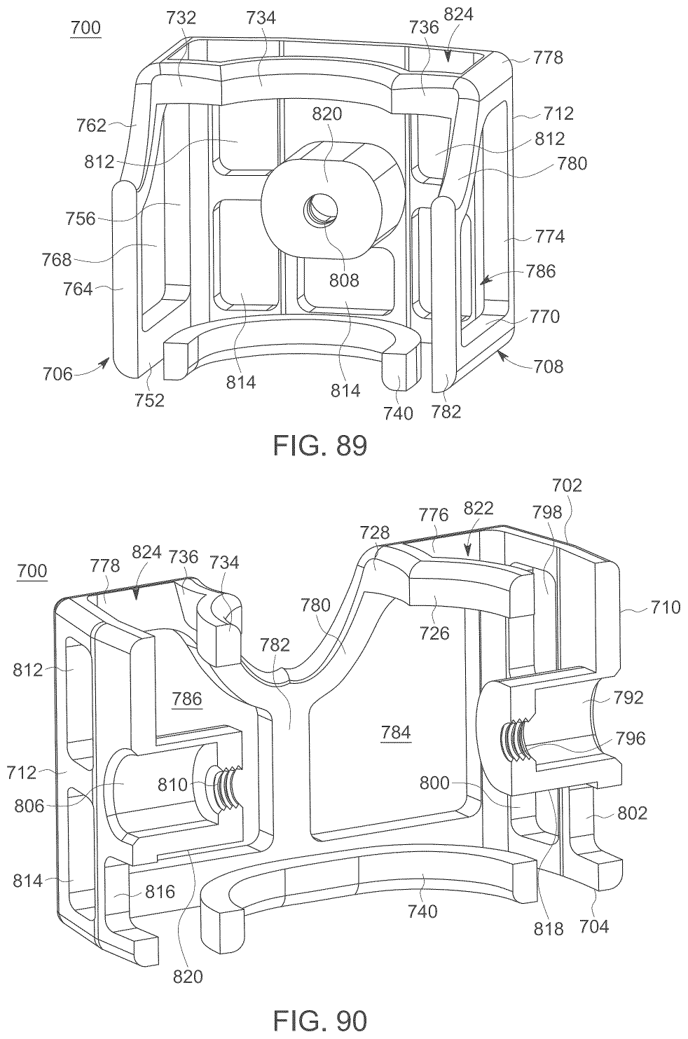

Referring now to FIGS. 81-90, another implant 700 is shown. The implant 700 may include a first side or top side 702, a second side or bottom side 704, a third side or right side 706, a fourth side or left side 708, a fifth side or first end 710, and a sixth side or second end 712. The first side 702 may extend, for example, generally parallel to the second side 704. The third side 706 may extend, for example, generally parallel to the fourth side 708. The fifth side 710 may extend, for example, generally parallel to the sixth side 712. The implant 700 may also include an interior frame 720 surrounded by an external frame or housing 750. The sides 702, 704, 706, 708, 710, 712 may form the external frame 750. The external frame 750 may have, for example, a rectangular prism shape.

With continued reference to FIGS. 81-90, the interior frame 720 may include a first intermediate portion 722 positioned at a top of the implant 700 between the first end 710 and a midpoint, a second intermediate portion 730 positioned at a bottom of the implant 700 between the midpoint and the second end 712, and a third intermediate portion or bottom member 740. The first intermediate portion 722 includes a first segment 724, a second segment 726, and a third segment 728. A first end of the first segment 724 is coupled to the third side 706 of the implant 700. The second segment 726 is coupled to the first segment 724 on a first end. A first end of the third segment 728 is coupled to a second end of the second segment 726 and a second end of the third segment 728 is coupled to the fourth side 708 of the implant 700. Thus, the first intermediate portion 722 extends between the third side 706 and the fourth side 708 of the implant 700. Each of the first, second, and third segments 724, 726, 728 may be, for example, curved between the first and second ends of each segment 724, 726, 728. The segments 724, 726, 728 may include, for example, a concave surface that is positioned to face the through hole 738. The second intermediate portion 730 includes a fourth segment 732, a fifth segment 734, and a sixth segment 736. A first end of the fourth segment 732 is coupled to the third side 706 of the implant 700. The fifth segment 734 is coupled to the fourth segment 732 on a first end. A first end of the sixth segment 736 is coupled to a second end of the fifth segment 734 and a second end of the sixth segment 736 is coupled to the fourth side 708 of the implant 700. Thus, the second intermediate portion 730 extends between the third side 706 and the fourth side 708 of the implant 700. Each of the fourth, fifth and sixth segments 732, 734, 736 may be, for example, curved between the first and second ends of each segment 732, 734, 736. The segments 732, 734, 736 may include, for example, a concave surface that is positioned to face the through hole 738. The bottom member 740 is positioned, for example, aligned with the bottom side 704 of the implant 700. In addition, the bottom member 740 is positioned, for example, between the third side 706 and fourth side 708 and between the fifth side 710 and the sixth side 712. The through hole 738 also extends through the bottom member 740. The bottom member 740 may have, for example, a circular, oval or round shape. The bottom member 740 may be, for example, sized and shaped to allow for an inserted crossing screw or intramedullary nail to pass through the bottom side 704 of the implant 700.

The external frame or housing 750 may include supports 752, 754, 756, 758, 760, 762, 764 on the third side 706 of the implant 700, as shown in FIG. 86. The first support, first base or first linear member 752 may be positioned, for example, at an inferior end of the third side 706, i.e. where the third side 706 engages the second side 704. The first arm, second linear member or second support 754 may extend from a first end of the first support 752, for example, perpendicular to the first support 752. The first arm 754 may be positioned, for example, where the third side 706 engages the fifth side 710. The second arm, third linear member or third support 756 may extend from a second end of the first support 752, for example, perpendicular to the first support 752. The fourth linear member or fourth support 758 may extend from a first end of the first arm 754 toward the through hole 738 of the interior frame 720. The fifth linear member or fifth support 760 may extend from a first end of the second arm 756 toward the through hole 738 of the interior frame 720. The first guide hole member or sixth support 762 may, for example, extend between the fourth linear member 758 and the fifth linear member 760. The first guide hole member 762 may be, for example, curved. The curvature of the first guide hole member 762 may be, for example, a concave curvature. The first guide hole member 762 may be, for example, sized and shaped to allow for a crossing screw to be inserted through the implant 700 at an angle that extends past the first guide hole member 762 during insertion from the third side 706. The third arm, sixth linear member or seventh support 764 may be coupled to an intermediate portion of the first guide hole member 762 on a first end and the first base 752 on a second end. The third arm 764 may, for example, couple to the distal most portion of the first guide hole member 762. The first arm 754, second arm 756 and third arm 764 may each, for example, extend parallel to each other as the arms 754, 756, 764 extend from the bottom side 704 toward the top side 702. The third side 706 may also include at least one first window 766 and at least one second window 768, as shown in FIGS. 82 and 86. The at least one first window 766 may be positioned, for example, intermediate a portion of the first base 752, first arm 754, fourth linear member 758, a first portion of the first guide hole member 762, and the third arm 764. Thus, the first base 752, first arm 754, fourth linear member 758, a first portion of the first guide hole member 762, and the third arm 764 provide the surrounding structure to form the at least one first window 766 extending through the third side 706. The at least one second window 768 may be positioned, for example, intermediate a portion of the first base 752, the second arm 756, fifth linear member 760, a second portion of the first guide hole member 762, and the third arm 764. Thus, the first base 752, the second arm 756, the fifth linear member 760, a second portion of the first guide hole member 762, and the third arm 764 provide the surrounding structure to form the at least one second window 768 extending through the third side 706.

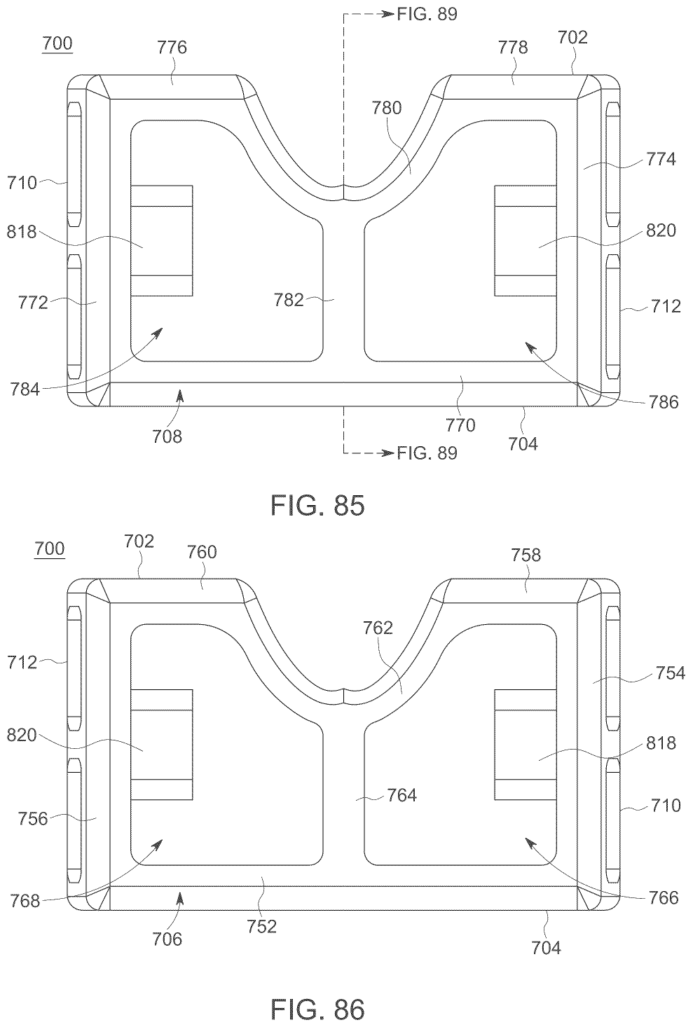

Referring now to FIG. 85, the interior frame 720 may also include supports 770, 772, 774, 776, 778, 780, 782 on the fourth side 708 of the implant 700. The eighth support, second base or seventh linear member 770 may be positioned, for example, at an inferior end of the fourth side 708, i.e. where the fourth side 708 engages the second side 704. The fourth arm, eighth linear member or ninth support 772 may extend from a first end of the eighth support 770, for example, perpendicular to the eighth support 770. The fourth arm 772 may be positioned, for example, where the fourth side 708 engages the fifth side 710. The fifth arm, ninth linear member or tenth support 774 may extend from a first end of the second base 770, for example, perpendicular to the second base 770. The tenth linear member or eleventh support 776 may extend from a first end of the fourth arm 772 toward the through hole 738 of the interior frame 720. The eleventh support 776 may extend between the fourth arm 772 and the third segment 728 of the first intermediate portion 722. The eleventh linear member or twelfth support 778 may extend from a first end of the fifth arm 774 toward the through hole 738 of the interior frame 720. The twelfth support 778 may extend between the fifth arm 774 and the sixth segment 736 of the second intermediate portion 730. The second guide hole member or thirteenth support 780 may, for example, extend between the tenth linear member 776 and the eleventh linear member 778. The second guide hole member 780 may be, for example, curved. The curvature of the second guide hole member 780 may be, for example, a concave curvature. The second guide hole member 780 may be, for example, sized and shaped to allow for a crossing screw to be inserted through the implant 700 at an angle that extends past the second guide hole member 780 during insertion from the fourth side 708. The sixth arm, twelfth linear member or fourteenth support 782 may be coupled to an intermediate portion of the second guide hole member 780 on a first end and the second base 770 on a second end. The sixth arm 782 may, for example, couple to the distal most portion of the second guide hole member 780. The fourth arm 772, fifth arm 774 and sixth arm 782 may each, for example, extend parallel to each other as the arms 772, 774, 782 extend from the bottom side 704 toward the top side 702. The fourth side 708 may also include at least one third window 784 and at least one fourth window 786, as shown in FIGS. 81 and 85. The at least one third window 784 may be positioned, for example, intermediate a portion of the second base 770, the fourth arm 772, the tenth linear member 776, a first portion of the second guide hole member 780, and the sixth arm 782. Thus, the second base 770, fourth arm 772, tenth linear member 776, first portion of the second guide hole member 780, and the sixth arm 782 provide the surrounding structure to form the at least one third window 784 extending through the fourth side 708. The at least one fourth window 786 may be positioned, for example, intermediate a portion of the second base 770, the fifth arm 774, the eleventh linear member 778, a second portion of the second guide hole member 780, and the sixth arm 782. Thus, the second base 770, fifth arm 774, eleventh linear member 778, second portion of the second guide hole member 780, and sixth arm 782 provide the surrounding structure to form the at least one fourth window 786 extending through the fourth side 708.

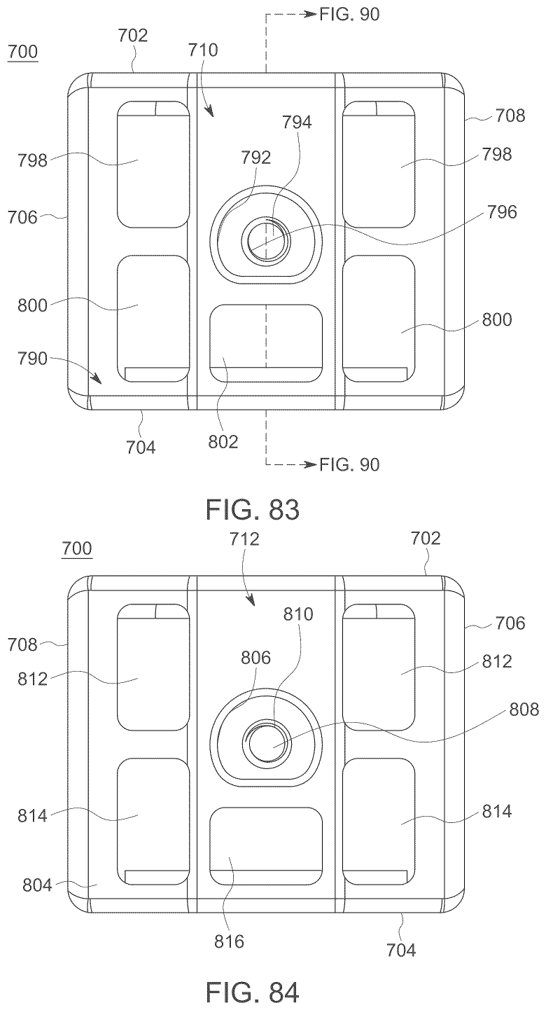

Referring now to FIG. 83, the external frame 750 may also include a first support member 790 on the fifth side 710 of the implant 700. The first support member 790 may include a first recessed region 792 extending into an intermediate portion from the fifth side 710. The first recessed region 792 may have, for example, a length, width and height and the length may be larger than the height. For the first recessed region 792, the height may extend in a direction between the top side 702 and the bottom side 704, the length may extend in a direction between the third side 706 and the fourth side 708, and the width may extend in a direction between the first end 710 and the second end 712. The first recessed region 792 may have, for example, a flat bottom surface and be rounded or curved between a first end and a second end of the flat bottom surface. The first recessed region 792 may be, for example, oval, elliptical, or sideways D shaped. The first recessed region 792 may have, for example, a non-circular shape. The first support member 790 may also include a first through hole 794 extending through the first support member 790. The first through hole 794 may extend, for example, entirely through the fifth side 710 of the implant 700. The first through hole 794 may include, for example, threads 796 on the interior surface.

The first support member 790 may also include at least one fifth window, superior window, or opening 798 extending through the first support member 790. The at least one fifth window 798 may include, for example, two parallel sides, a top portion connecting the two parallel sides at a first end, and a base portion connecting the two parallel sides at a second end. The top portion and bottom portion may be, for example, parallel to each other as they extend between the two sides. The at least one fifth window 798 may be, for example, at least two windows 798 positioned on a medial and lateral side of the fifth side 710. As shown in the depicted embodiment, the two fifth windows 798 may be positioned to be mirror images of each other and near a superior end the implant 700.

The first support member 790 may also include at least one sixth window, inferior window, or opening 800 extending through the first support member 790, as shown in FIG. 83. The at least one sixth window 800 may include, for example, two parallel sides, a top portion connecting the two parallel sides at a first end, and a base portion connecting the two parallel sides at a base end. The top portion and bottom portion may be, for example, parallel to each other as they extend between the two sides. The at least one sixth window 800 may be, for example, at least two windows 800 positioned on a medial and lateral side of the fifth side 710. As shown in the depicted embodiment, the two sixth windows 800 may be positioned to be mirror images of each other and near an inferior end of the fifth side 710 of the implant 700. The first support member 790 may also include at least one seventh window, intermediate window or opening 802. The at least one seventh window 802 may be positioned, for example, adjacent to the first recessed region 792. In the depicted embodiment, the at least one seventh window 802 is one seventh window 802 that is positioned inferior to the recessed region 792 and between the recessed region 792 and the bottom side 704 of the implant 700.

Referring now to FIG. 84, the frame 750 may include a second support member 804 on the sixth side 712 of the implant 700. The second support member 804 may include a second recessed region 806 extending into an intermediate portion from the sixth side 712. The second recessed region 806 may have, for example, a length, width and height and the length may be larger than the height. The height may extend in a direction between the top side 702 and the bottom side 704, the length may extend in a direction between the third side 706 and the fourth side 708, and the width may extend in a direction between the first end 710 and the second end 712. The second recessed region 806 may have, for example, a flat bottom surface and be rounded or curved between a first end and a second end of the flat bottom surface. The second recessed region 806 may be, for example, oval, elliptical, or sideways D shaped. The second recessed region 806 may have, for example, a non-circular shape. The second support member 804 may also include a second through hole 808 extending through the second support member 804. The second through hole 808 may extend, for example, entirely through the sixth side 712 of the implant 700. The second through hole 808 may include, for example, threads 810 positioned on the interior surface of the second through hole 808.

The second support member 804 may also include at least one eighth window, superior window, or opening 812 extending through the second support member 804. The at least one eighth window 812 may include, for example, two parallel sides, a top portion connecting the two parallel sides at a first end, and a base portion connecting the two parallel sides at a second end. The top portion and base portion may be, for example, positioned parallel to each other as they extend between the two sides. The at least one eighth window 812 may be, for example, at least two windows 812 positioned on a medial and lateral side of the sixth side 712. As shown in the depicted embodiment, the two eighth windows 812 may be positioned to be mirror images of each other and near a superior end the implant 700.

The second support member 804 may also include at least one ninth window, inferior window, or opening 814 extending through the second support member 804. The at least one ninth window 814 may include, for example, two parallel sides, a top portion connecting the two parallel sides on a first end, and a base portion connecting the two parallel sides on a second end. The top portion and base portion may be, for example, parallel to each other as they extend between the two sides. The at least one ninth window 814 may be, for example, at least two windows 814 positioned on a medial and lateral side of the sixth side 712. As shown in the depicted embodiment, the two ninth windows 814 may be positioned to be mirror images of each other and near an inferior end of the sixth side 712 of the implant 700. The second support member 804 may also include at least one tenth window, intermediate window or opening 816. The at least one tenth window 816 may be positioned, for example, adjacent to the second recessed region 806. In the depicted embodiment, the at least one tenth window 816 is, for example, one tenth window 816 that is positioned inferior to the second recessed region 806 and between the second recessed region 806 and the bottom side 704. In the depicted embodiment, the windows 798, 800, 802, 812, 814, 816 may be, for example, square or rectangularly shaped, although alternative shapes are also contemplated as disclosed above with reference to implant 100, which will not be described again here for brevity sake.

Referring now to FIGS. 81 and 85-90, the frame 750 may also include a first protrusion, interior protrusion or first screw housing 818 extending away from an interior surface of the fifth side 710. The first protrusion 818 may be positioned, for example, intermediate the first side 702 and second side 704, as well as the third side 706 and the fourth side 708. The first protrusion 818 may have, for example, a cylindrical or elliptic cylindrical shape as the first protrusion 818 extends from the fifth side 710 toward the sixth side 712, as shown in FIGS. 81, 89, and 90. The first through hole 794 may extend through the first protrusion 818.

Referring now to FIGS. 82 and 87-90, the frame 750 may include a second protrusion, interior protrusion or second screw housing 820 extending away from an interior surface of the sixth side 712. The second protrusion 820 may be positioned, for example, intermediate the first side 702 and second side 704, as well as the third side 706 and the fourth side 708. The second protrusion 820 may have, for example, a cylindrical or elliptic cylindrical shape as the second protrusion 820 extends from the sixth side 712 toward the fifth side 710, as shown in FIGS. 82 and 90. The second through hole 808 may extend through the second protrusion 820.

With continued reference to FIGS. 81 and 87-90, the implant 700 may also include a first passageway 822 extending between the external frame 750 and the interior frame 720 near the first end 710. The first passageway 822 is open from the first side 702 to the second side 704. The first through hole 794 and the openings 798, 800, 802 extend from the exterior surface of the frame 750 through to the passageway 822. The implant 700 may further include a second passageway 824 extending between the exterior frame 750 and the interior frame 720 near the second end 712. The second passageway 824 is open from the first side 702 to the second side 704. The second through hole 808 and the openings 812, 814, 816 extend from the exterior surface of the frame 750 through to the passageway 824.

Although not shown, the implant 700 may also include a lattice structure positioned within the openings 766, 768, 784, 786, 798, 800, 802, 812, 814, 816, 822, 824 of the implant 700. The lattice structure may be positioned, for example, within the first and second passageways 822, 824 to assist with bone fusion. The lattice structure may be, for example, planar with the sides 702, 704, 703, 708, 710, 712 of the implant 700 or recessed into the implant 700 from the exterior surface of the sides 702, 704, 703, 708, 710, 712. The lattice structure may be, for example, printed within the first and second passageways 822, 824. The lattice structure may be, for example, non-nodal randomized web based matrix. The lattice structure matrix may have a pore size of, for example, approximately 4 mm to approximately 5 mm. The lattice structure may include, for example, rough elements that span between the structure that forms the passageways 822, 824. The lattice structure may be, for example, offset from the outer frame of implant 700. The lattice structure may be offset inside of the frame of the implant 700 by approximately 0.5 mm. Thus, the frame of the implant 700 is the only bone contacting element once implanted and the lattice structure does not directly contact bone.

The implant 700 may be inserted into a patient's ankle as described in greater detail above with respect to the surgical method for the implant 100, which will not be described again here for brevity sake. Specifically, the implant 700 may be inserted into a patient using an anterior approach. In addition, the implant 700 may use a cross-screw, a cross-screw and plate, or an intramedullary nail to fix the implant 700 in the patient's ankle.

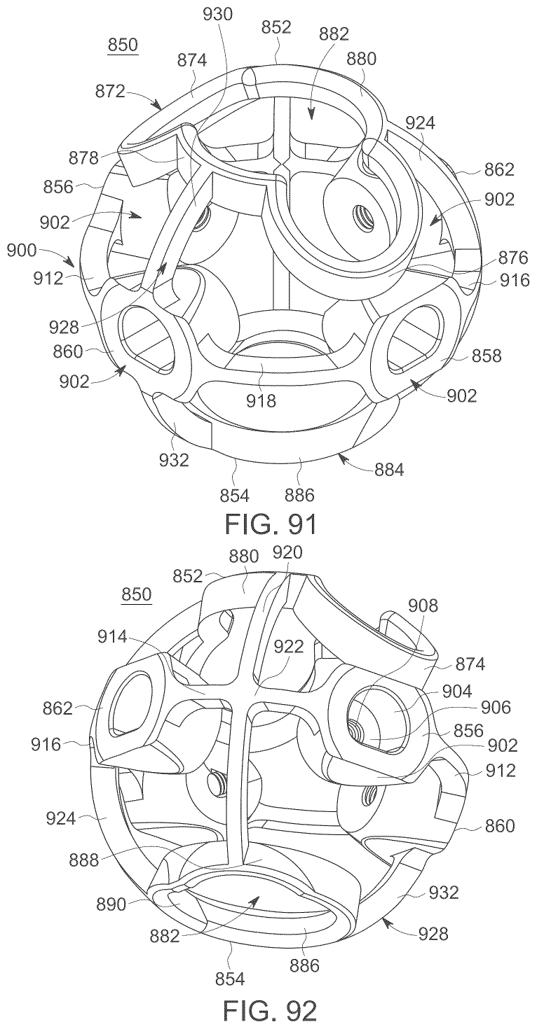

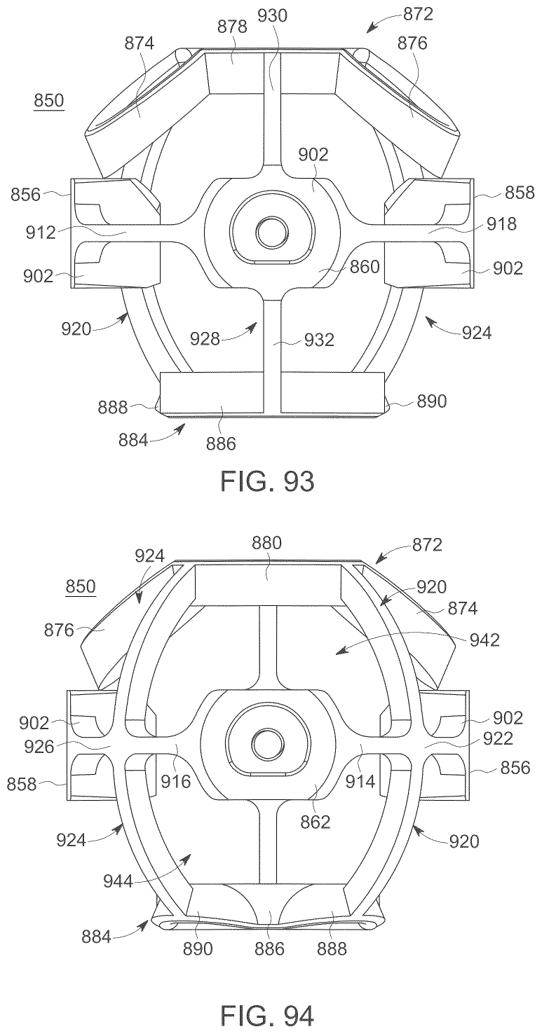

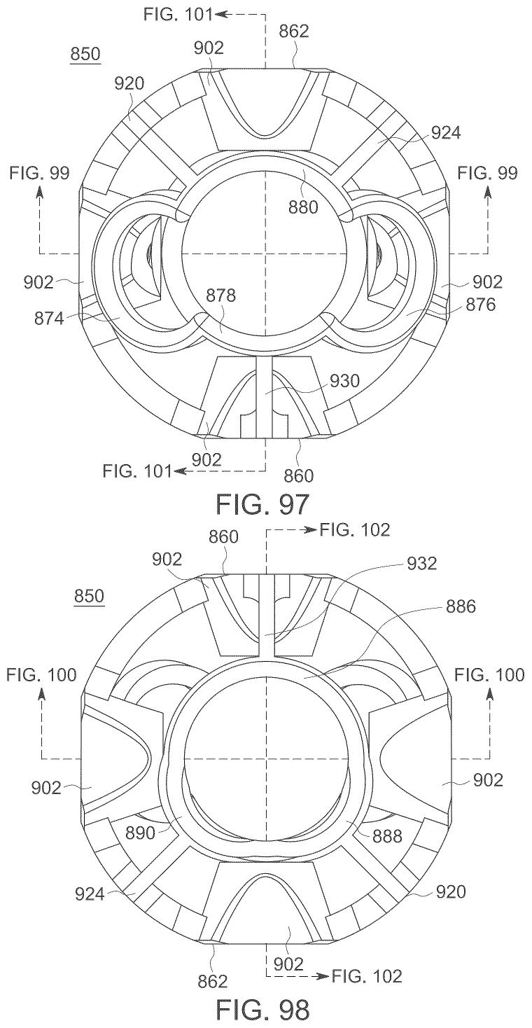

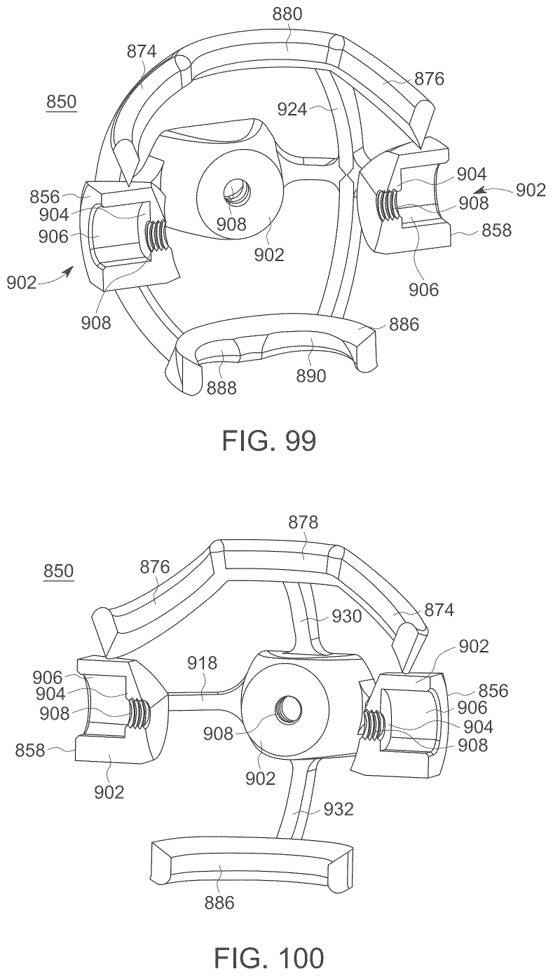

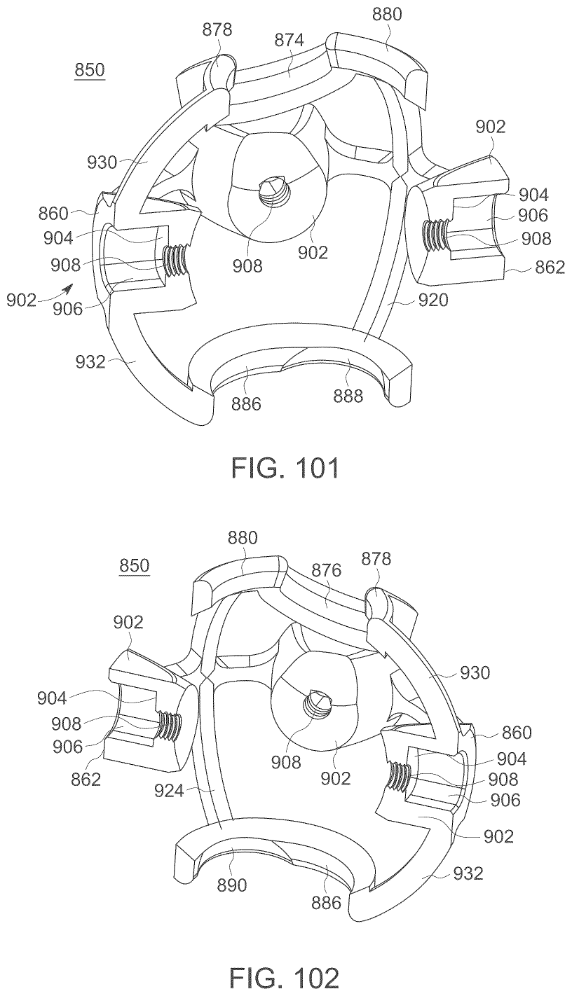

Referring now to FIGS. 91-102, another implant 850 is shown. The implant 850 may include a first side or top side 852, a second side or bottom side 854, a third side or right side 856, a fourth side or left side 858, a fifth side or first end 860, and a sixth side or second end 862. The first side 852 and the second side 854 may each be, for example, curved in opposite directions giving the implant 850 a generally round or ball shape. The third side 856 and the fourth side 858 may also each be, for example, curved in opposite directions. In addition, the fifth side 860 and the sixth side 862 may each be, for example, curved in opposite directions. The implant 850 may also include an interior frame 870 surrounded by or positioned within an external frame or housing 900. The external frame 900 may have, for example, a spherical shape.

The interior frame 870 may include a first intermediate portion 872 and a second intermediate portion 884, as shown in FIGS. 91-102. The first intermediate portion 872 may be, for example, positioned at the top side 852 of the implant 850 and the second intermediate portion 884 may be, for example, positioned at the bottom side 854 of the implant 850. The first intermediate portion 872 may include a first ring segment or first lobe 874, a second ring segment or second lobe 876, and two connecting segments 878, 880 extending between the first lobe 874 and the second lobe 876. The first intermediate portion 872 may be, for example, curved as it extends between the third side 856 and the fourth side 858. The first lobe 874 is positioned on the third side 856 of the implant 850 and the second lobe 876 is positioned on the fourth side 858. The lobes 874, 876 may be, for example, curved from their point of connection with the connecting segments 878, 880 as they extend to the third and fourth sides 856, 858, respectively. The lobes 874, 876 may be, for example, horseshoes, open rings or U-shaped segments coupled together by the connecting segments 878, 880 to form a through hole 882. The through hole 882 may have, for example, three overlapping circles, a first circle formed by the first lobe 874, a second circle formed by the first and second connecting segments 878, 880, and a third circle formed by the second lobe 876. A first end of the first connecting segment 878 is coupled to a first end of the first lobe 874 and a second end of the first connecting segment 878 is coupled to a first end of the second lobe 876. A first end of the second connecting segment 880 is coupled to a second end of the first lobe 874 and a second end of the second connecting segment 880 is coupled to a second end of the second lobe 876.

With continued reference to FIGS. 91-102, the second intermediate portion 884 may include a body 886 with a first lobe 888 and a second lobe 890. The body 886 may be, for example, positioned at the second side 854 of the implant 850. The first lobe 888 may extend away from a portion of the body 886 intermediate the third side 856 and sixth side 862 of the implant 850. The second lobe 890 may extend away from a portion of the body 886 intermediate the fourth side 858 and the sixth side 862. The first lobe 888 may be spaced apart from the second lobe 890 between the third and fourth sides 856, 858 on the sixth side 862. The body 886, the first lobe 888, and the second lobe 890 may form an exterior structure surrounding the through hole 882 that extends from the first intermediate portion 872. The through hole 882 extends through the implant 850 to receive a crossing screw or intramedullary nail. The first lobe 874 of the first intermediate portion 872 may be angled to align with the second lobe 890 of the second intermediate portion 884 in a first direction. The second lobe 876 of the first intermediate portion 872 may be angled to align with the first lobe 888 of the second intermediate portion 884 in a second direction. The first direction and the second direction may include trajectories extending in opposite directions. The first trajectory extending between the first lobe 874 and the second lobe 890 may overlap the second trajectory extending between the second lobe 876 and the first lobe 888 as the trajectories extend through the implant 850 from a first side 852 to a second side 854. The positioning of the lobes 874, 876, 888, 890 enables, for example, a crossing screw to be inserted on an angle as it passes through the joint.

As shown in FIGS. 91-102, the external frame 900 may include a plurality of coupling portions 902 and a plurality of support arms 912, 914, 916, 918 coupled to and connecting the coupling portions 902. The coupling portions 902 may be positioned, for example, on the third side 856, fourth side 858, fifth side 860, and sixth side 862. The coupling portion 902 positioned on the third side 856 is opposite the coupling portion 902 positioned on the fourth side 858. The coupling portion 902 positioned on the fifth side 860 is opposite the coupling portion 902 positioned on the sixth side 862. The first support arm 912 may extend between and couple the coupling portion 902 on the fifth side 860 to the coupling portion 902 on the third side 856. The second support arm 914 may extend between and couple the coupling portion 902 on the third side 856 to the coupling portion 902 on the sixth side 862. The third support arm 916 may extend between and couple the coupling portion 902 on the sixth side 862 to the coupling portion 902 on the fourth side 858. Finally, the fourth support arm 918 may extend between and couple the coupling portion 902 on the fourth side 858 to the coupling portion 902 on the fifth side 860. The coupling portions 902 and arms 912, 914, 916, 918 of the external frame 900 together form a ring around the interior frame 870.

The external frame 900 may also include crossing members 920, 924, 928 coupling the first intermediate portion 872, the second intermediate portion 884, and at least one of the coupling portions 902 and/or arms 912, 914, 916, 918. For example, as shown in FIGS. 92-95, 97-99 and 101-102, a first crossing member 920 may couple to the first intermediate portion 872 on a first end, extend through the second arm 914, and couple to the second intermediate portion 884 on a second end. The first crossing member 920 extends over the second arm 914 forming a first node 922. The first node 922 forms an enlarged portion near a midpoint of the second arm 914. The second crossing member 924 may couple to the first intermediate portion 872 on a first end, extend through the third arm 916, and couple to the second intermediate portion 884 on a second end. The second crossing member 924 extends over the third arm 916 forming a second node 926. The second node 926 forms an enlarged portion near a midpoint of the third arm 916. The third crossing member 928 may couple to the first intermediate portion 872 on a first end, engage two sides of a coupling portion 902 near a midpoint, and couple to the second intermediate portion 884 on a second end. As shown in FIGS. 91-96 and 100, the third crossing member 928 engages the coupling portion 902 on the fifth side 860 of the implant 850. The third crossing member 928 may include two supports 830, 832. The two supports may be a seventh support 830 and an eighth support 832. The seventh support 830 may couple to the first intermediate portion 872 on a first end and the coupling portion 902 on a second end, while the eighth support 832 may couple to the coupling portion 902 on a first end and the second intermediate portion 884 on a second end, as shown in FIGS. 91, 93, 95, 96 and 100. The crossing members 920, 924, 928 couple the interior frame 870 to the external frame 900. In addition, the crossing members 920, 924, 928 may be positioned spaced apart around the implant 850. The spacing of the crossing members 920, 924, 928 may be, for example, equally spaced around the exterior surface of the implant 850 or unequally spaced around the exterior surface of the implant 850.

The coupling portions 902 may have a first height extending in a direction between the first side 852 and the second side 854 and a first width extending from an exterior surface to an interior surface of the implant 850. The arms 912, 914, 916, 918 may have a second height extending in a direction between the first side 852 and the second side 854 and a second width extending from an exterior surface to an interior surface of the implant 850. The first height of the coupling portions 902 may be, for example, larger than the second height of the arms 912, 914, 916, 918. The first width of the coupling portions 902 may be, for example, the same or larger than the second width of the arms 912, 914, 916, 918.

Each of the coupling portions 902 may also include a recessed region 904 inset into the coupling portions 902 from the exterior surface towards an interior surface of the external frame 900. The recessed region 904 may extend from the exterior surface of the implant 850 into the coupling portions 902 to an interior surface 906 of the recessed region 904. The recessed region 904 may have, for example, a flat bottom surface and be rounded or curved between a first end and a second end of the flat bottom surface. The recessed region 904 may have, for example, an oval, elliptical, or sideways D shape. In addition, the coupling portions 902 may include a through hole 908 extending through each coupling portion 902 from the interior surface 906 of the recessed region 904 to the interior surface of the external frame 900. The through hole 908 may be positioned, for example, centered in the recessed region 904. Thus, the through hole 908 may overlap with the recessed region 904 in the coupling portions 902. The through hole 908 may be, for example, threaded to engage an inserter, such as inserter 550, which will not be described again here for brevity sake, or a like inserter with a threaded portion to engage the threads of the through hole 908.

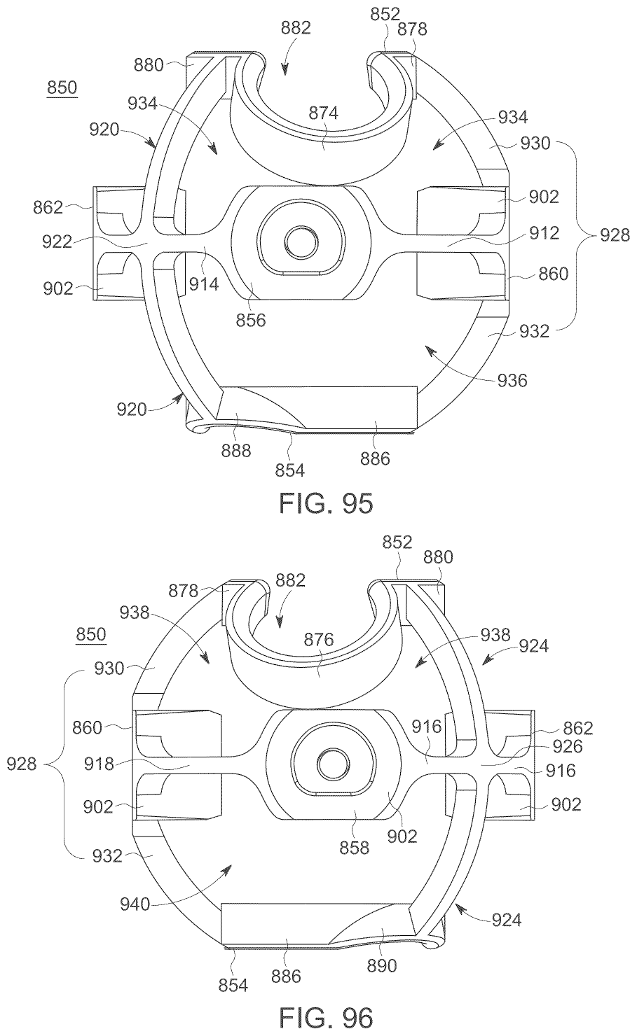

The implant 850 may also include openings 934, 936, 938, 940, 942, 944 extending through the implant between the interior frame 870 and external frame 900. The first opening 934 may extend between the first intermediate portion 872 and coupling portions 902, arms 912, 914 and crossing members 920, 928 of the external frame 900, as shown in FIG. 95. More specifically, the first opening 934 extends between the first arm 912, a portion of the second arm 914 extending between the coupling portion 902 on the third side 856 and the first node 922, a portion of the first crossing member 920 extending between the first node 922 and the second connecting segment 880, a portion of the second connecting segment 880 extending between the first crossing member 920 and the first lobe 876, a portion of the first connecting segment 878 extending between the first lobe 876 and the seventh support 930, and the seventh support 930. The second opening 936 may extend between the coupling portions 902, arms 912, 914, and crossing members 920, 928 of the external frame 900, as well as the second intermediate portion 884, as shown in FIG. 95. Specifically, the second opening 936 extends between a portion of the body 886 extending between the eighth support 932 of the third crossing member 928 and the first crossing member 920, a portion of the first crossing member 920 extending between the first lobe 888 of the second intermediate portion 884 and the coupling portion 902 on the sixth side 862, the second arm 914 between first node 922 and the coupling portion 902 on the third side 856, the first arm 912, and the eighth support 932 of the third crossing member 928. The third opening 938 may extend between the coupling portions 902, arms 916, 918, and crossing members 924, 928 of the external frame 900, as well as the first intermediate portion 872, as shown in FIG. 96. Specifically, the third opening 938 extends between a portion of the third arm 916 extending between the second node 926 and the coupling portion 902 on the fourth side 858, the fourth arm 918, the seventh support 930 of the third crossing member 928, a portion of the first connecting segment 878 extending between the seventh support 930 and the second lobe 876, the second lobe 876, a portion of the second connecting segment 880 extending between the second lobe 876 and the first end of the second crossing member 924, and a portion of the second crossing member 924 extending from the second connecting segment 880 and the second node 926. The fourth opening 940 may extend between the coupling portions 902, arms 916, 918, and crossing members 924, 926 of the external frame 900, as well as the second intermediate portion 884, as shown in FIG. 96. Specifically, the fourth opening 940 extends between a portion of the body 886 extending between the second crossing member 924 and the eighth support 932 of the third crossing member 928, the fourth arm 918, a coupling portion 902 on the fourth side 858 of the implant 850, a portion of the third arm 916 extending between the coupling portion 902 and a second node 926, and a portion of the second crossing member 924 extending between the second node 926 and the second intermediate portion 884. The fifth opening 942 may extend between the fourth coupling portion 902 on the sixth side 862, portions of the arms 914, 916, portions of the crossing members 920, 924, and the second connecting segment 880, as shown in FIG. 94. Specifically, the fifth opening 942 extends between a portion of the second connecting segment 880 between the first crossing member 920 and the second crossing member 924, a portion of the first crossing member 920 extending between the second connecting segment 880 and the first node 922, a portion of the second arm 914 extending between the first node 922 and the fourth coupling portion 902, the fourth coupling portion 902, a portion of the third arm 916 extending between the fourth coupling portion 902 and the second node 926, and a portion of the second crossing member 924 extending between the second node 926 and the second connecting segment 880. The sixth opening 944 may extend between the fourth coupling portion 902, portions of the arms 914, 916, portions of the crossing members 920, 924, and the second intermediate portion 884, as shown in FIG. 94. Specifically, the sixth opening 944 extends between a portion of the second arm 914 extending between the first node 922 and the fourth coupling portion 902, the fourth coupling portion 902, a portion of the third arm 916 extending between the fourth coupling portion 902 and the second node 926, a portion of the second crossing member 924 extending between the second node 926 and the second lobe 890 of the second intermediate portion 884, the body 886 between the second crossing member 924 and the first crossing member 902, and a portion of the first crossing member 902 extending between the first lobe 888 of the second intermediate portion 884 and the first lobe 922.

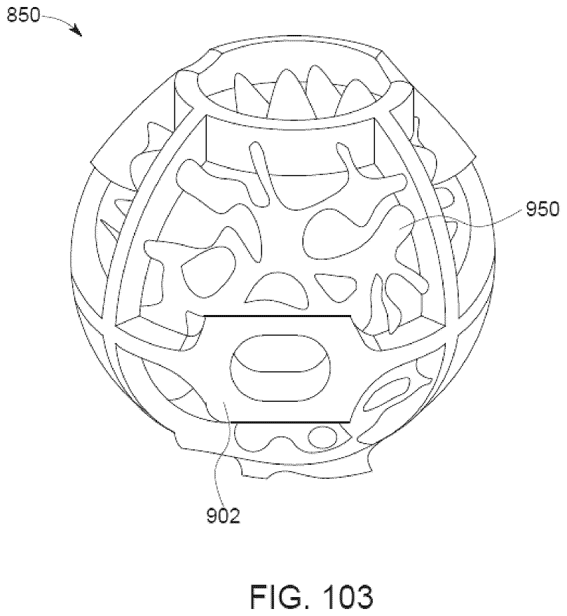

As shown in FIG. 103, the implant 850 may also include a lattice structure 950 positioned within the openings 934, 936, 938, 940, 942, 944 of the implant 850. The lattice structure 950 may be, for example, the same or similar to any other lattice structure shown and described in the present application. In addition, the lattice structure 950 may be, for example, made up of randomly organized elements. The lattice structure 950 may also have, for example, varying surface roughness across the elements that make up the lattice structure 950. Further, the lattice structure 950 may not include, for example, defined nodes and/or linear struts. The lattice structure 950 may be, for example, offset from the outer frame of implant 850. The lattice structure 950 may be offset inside of the frame of the implant 850 by approximately 0.5 mm. Thus, the frame of the implant 850 is the only bone contacting element once implanted and the lattice structure 950 does not directly contact bone.

With continued reference to FIG. 103, the lattice structure 950 may be positioned, for example, within the openings 934, 936, 938, 940, 942, 944 to assist with bone fusion. The lattice structure 950 may be, for example, aligned with the spherical exterior surface on the sides 852, 854, 856, 858, 860, 862 of the implant 850 or recessed below the spherical exterior surface of the implant 850. The lattice structure 950 may have an outer boundary recessed, for example, approximately 0.5 mm-3 mm from the spherical exterior surface (e.g., recessed such that the outer boundary/boundaries of the lattice structure 950 is/are positioned radially inward from the spherical exterior surface of the implant). Further, the lattice structure may be recessed approximately 1 mm from the spherical exterior surface of the implant. In some aspects, the lattice structure 950 may be consistently recessed from the spherical outer surface as shown and described with reference to FIGS. 91-102, or may be variably recessed (e.g., different portions of the lattice structure 950 may be recessed variable distances from the spherical outer surface of the implant 850, with the radially-outermost point of the lattice structure 950 being recessed at least 0.5 mm from the spherical outer surface). The implant 850 may also be made, such that, the external frame 900 is proud relative to the lattice structure 950. In one embodiment, this may be accomplished by generating the lattice structure 950 into the external frame 900.

The lattice structure 950 may be, for example, printed within the openings 934, 936, 938, 940, 942, 944. The lattice structure 950 may be, for example, non-nodal randomized web based matrix. The lattice structure matrix 950 may have a pore size of, for example, approximately 4 mm to approximately 5 mm. The lattice structure 950 may also include, for example, rough elements that span between the structures that form the openings 934, 936, 938, 940, 942, 944.

The implant 850 may be inserted into a patient's ankle as described in greater detail above with respect to the surgical method for the implant 100, which will not be described again here for brevity sake. Specifically, the implant 850 may be inserted into a patient using any one of an anterior, lateral, or posterior approach. In addition, the implant 850 may use a cross-screw, a cross-screw and plate, or an intramedullary nail to fix the implant 850 in the patient's ankle.

Each lattice structure 230, 950 is a non-repeating, organically generated geometry. The terminations of the lattice structures 230, 950 are blended and randomized throughout the interior of the implants. The lattice structures 230, 950 can be, for example, formed to receive demineralized bone matrix, bone cement, or other biologics. In addition to the lattice structures, the implants disclosed herein may include one or more openings extending through the implants to allow for the passage of various fixation elements, for example, intramedullary nails, crossing screw, and the like. The one or more openings for receiving fixation elements may be, for example, positioned at an angle that is oblique relative to the vertical. Each of the implants disclosed herein could be customizable for a specific patient's anatomy. For patient specific implants the geometry of the implant may be designed to be complimentary to the anatomy of the patient, for example, to address a void, cyst or other abnormality in bones including the distal tibia.

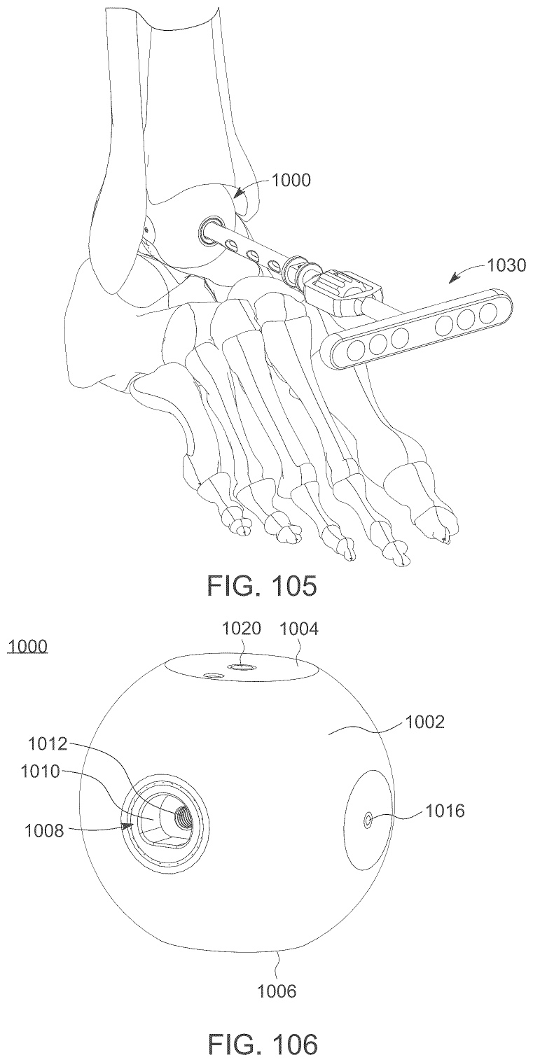

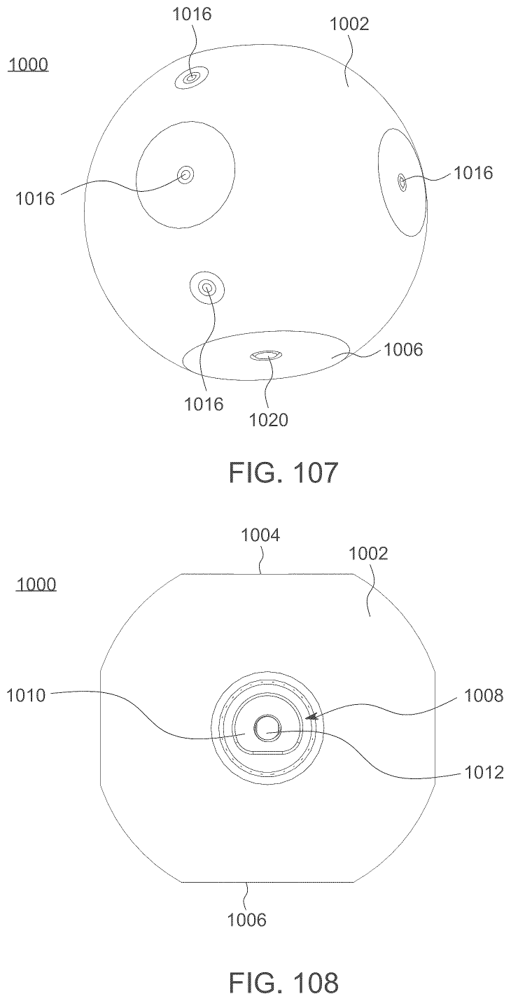

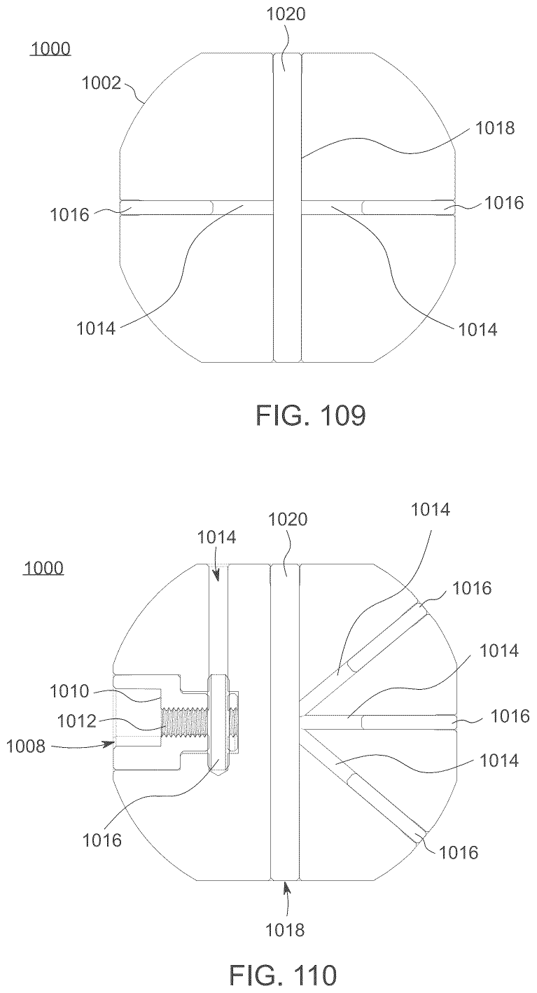

Referring now to FIGS. 105-110, an implant trial 1000 is shown. The implant trial 1000 includes an exterior surface 1002 having a spherical shape, a top surface 1004, and a bottom surface 1006. The top and bottom surfaces 1004, 1006 are, for example, flat surfaces positioned on opposite sides of the exterior surface 1002 from each other. The implant trial 1000 includes an inserter opening 1008 inset into the exterior surface 1002 of the trial 1000. The inserter opening 1008 includes a recessed region 1010 extending from the exterior surface 1002 into the trial 1000 and a through hole 1012 extending from the interior surface of the recessed region 1010 into the trial 1000. The through hole 1012 may be, for example, threaded to engage and couple to a threaded portion of an inserter 1030. The recessed region 1010 may have, for example, a size that is larger than the size of the through hole 1012 to receive an end of an inserter 1030. The trial 1000 may also include a plurality of holes 1014 for receiving radiopaque markers 1016. The openings for receiving an insertion instrument 550 in implants 100, 450, 700, 850 may include the inserter opening 1008 in place of the inserter openings described in greater detail above.

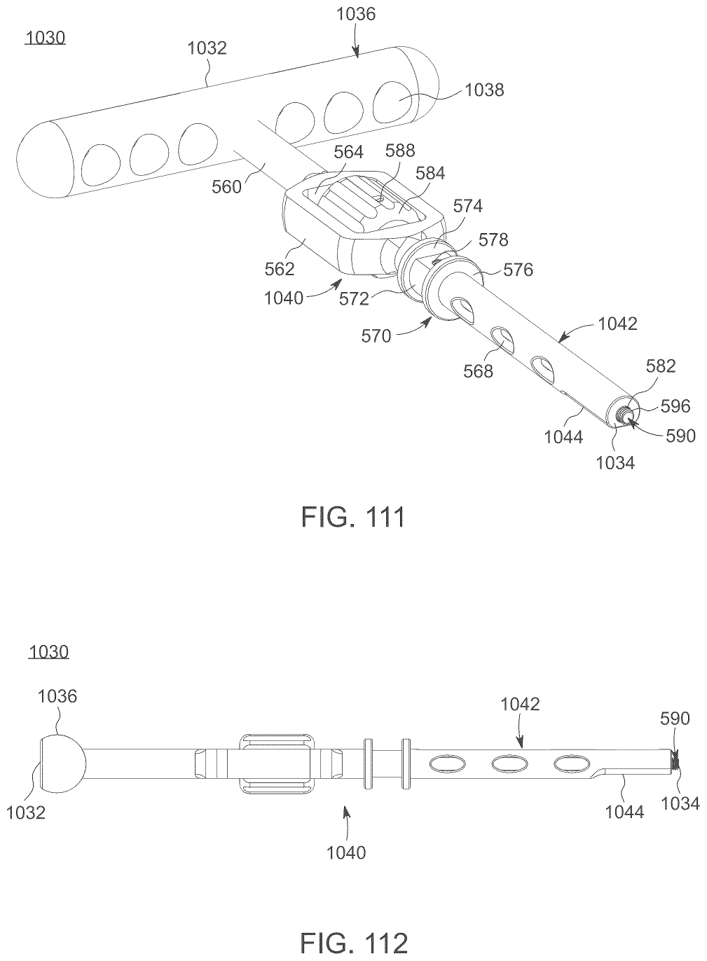

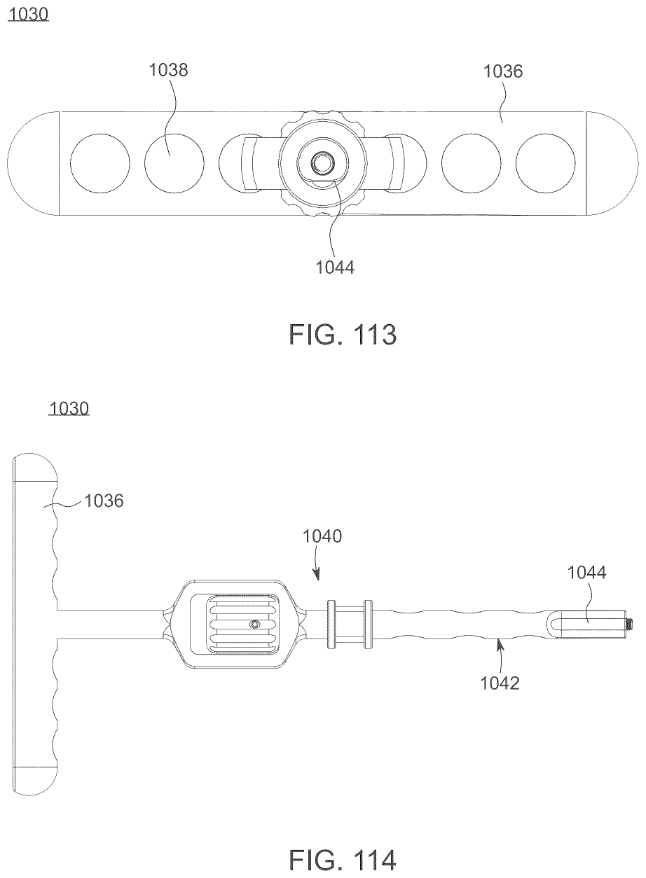

Referring now to FIGS. 111-114, another embodiment of an insertion instrument 1030 is shown. The insertion instrument 1030 is similar to the insertion instrument 550, as described in greater detail above. The instrument 1030 includes a first end 1032 and a second end 1034. The instrument 1030 also includes a handle 1036, a shaft 1040 coupled to the handle 1036, and an internal shaft 590 rotatably received within the shaft 1040. The internal shaft 590 is as described above with reference to instrument 550 and will not be described again here for brevity sake. The handle 1036 is positioned at a first end 1032 of the insertion instrument 1030. The handle 1036 includes a plurality of openings 1038 extending through the handle 1036 from the first end 1032 of the instrument towards the second end 1034 positioned on either side of the shaft 1040. The handle 1036 may include an opening for receiving the shaft 1040 or, alternatively, the shaft 1040 may be, for example, integrally coupled to the handle 1036. The shaft 1040 may include a first portion 560, a knob housing 562, and a second portion 1042. The first portion 560 is positioned at a first end of the shaft 1040 and couples to and extends from the handle 1036 toward the second end 1034 of the insertion instrument 1030. The knob housing 562 is coupled to the first portion 560 on a first end and the second portion 1042 on a second end of the knob housing 562. The first portion 560 and knob housing 562 are as described above with reference to instrument 550 and which will not be described again here for brevity's sake. The second portion 1042

The second portion 1042 extends from the second end of the knob housing 562 to toward the second end 1034 of the insertion instrument 1030. The second portion 1042 may include at least one through hole 568 extending through the second portion 1042 in a direction perpendicular to a longitudinal axis of the shaft 1040 extending between the first end 1032 and the second end 1034 of the insertion instrument 1030. The at least one through hole 568 may be, for example, aligned with the longest portion of the handle 1036. The at least one through hole 568 may be, for example, three through holes 568, although alternative numbers of through holes 568 are also contemplated. It is also contemplated that the through holes 568 may be positioned, for example, extending through the second portion 1042 in any orientation.

With continued reference to FIGS. 111-114, the second portion 1042 also includes a precision guide attachment portion 570. The attachment portion 570 may be as described above with reference to instrument 550 and which will not be described again here for brevity's sake. The first and second protrusions 574, 576 assist with retaining a guide, such as guides 1050, 1120, 1140, 1180 in the desired position along the shaft 1040 of the insertion instrument 1030. The attachment portion 570 also includes a hole 578 extending into the engagement surface 572. The hole 578 may extend into one side of the engagement surface 572 through at least a portion of the second portion 1042. The second portion 1042 may also include a recessed region 1044 on the second end of the shaft 1040. The recessed region 1044 may be, for example, positioned on a side of the second portion 1042 perpendicular to the opening 564. The recessed region 1044 may be, for example, tapered, curved or arched from a position offset from the second end 1034 of the insertion instrument 1030 and extending to the second end of the second portion 1042. In addition, the shaft 1040 may include a passageway 582 extending from the second end of the second portion 1042 of the shaft 1040 to the opening 564 in the knob housing 562. The passageway 582 may be, for example, sized and shaped to receive the internal shaft 590.

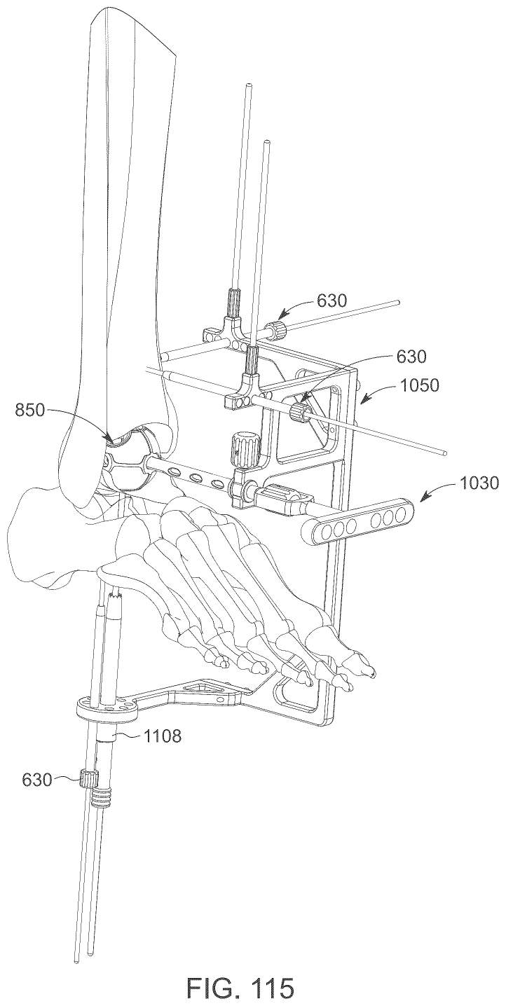

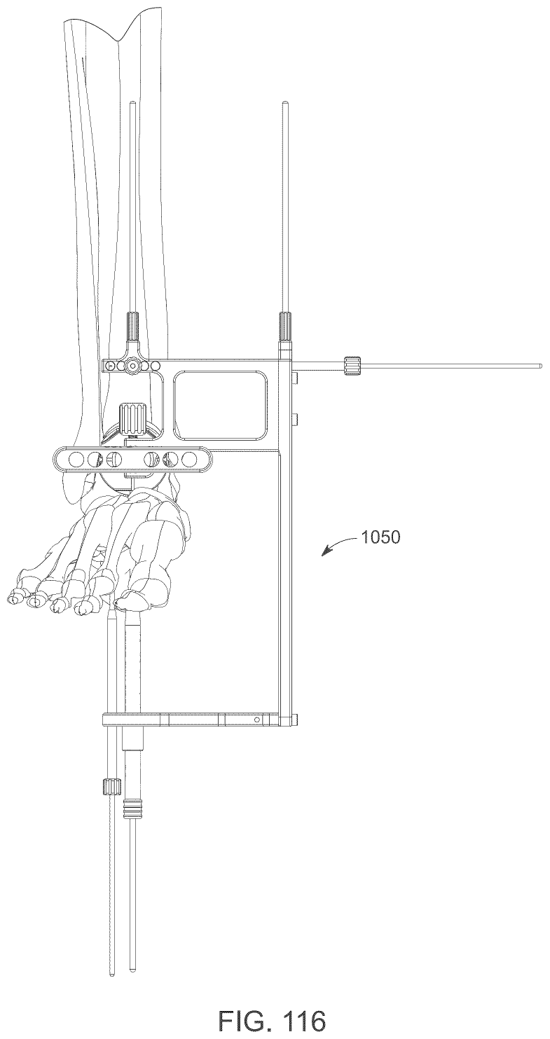

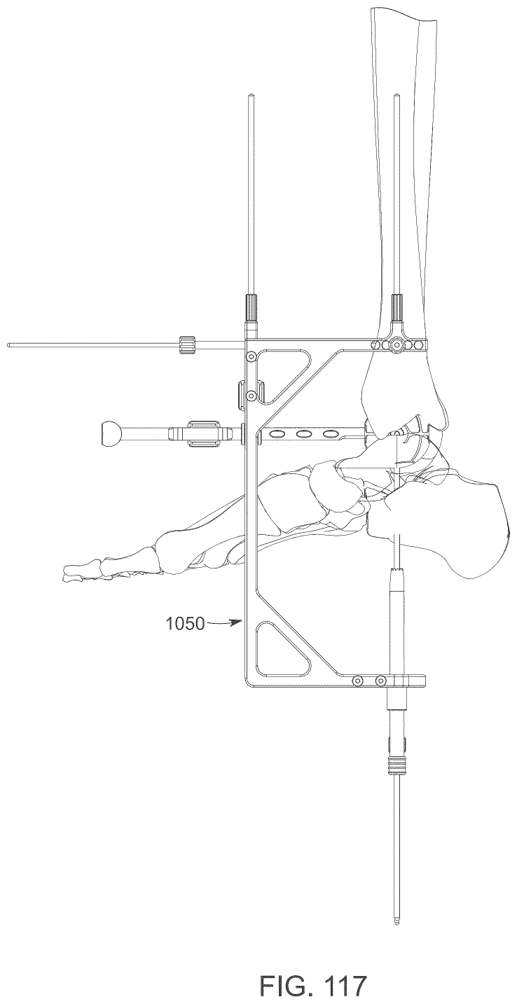

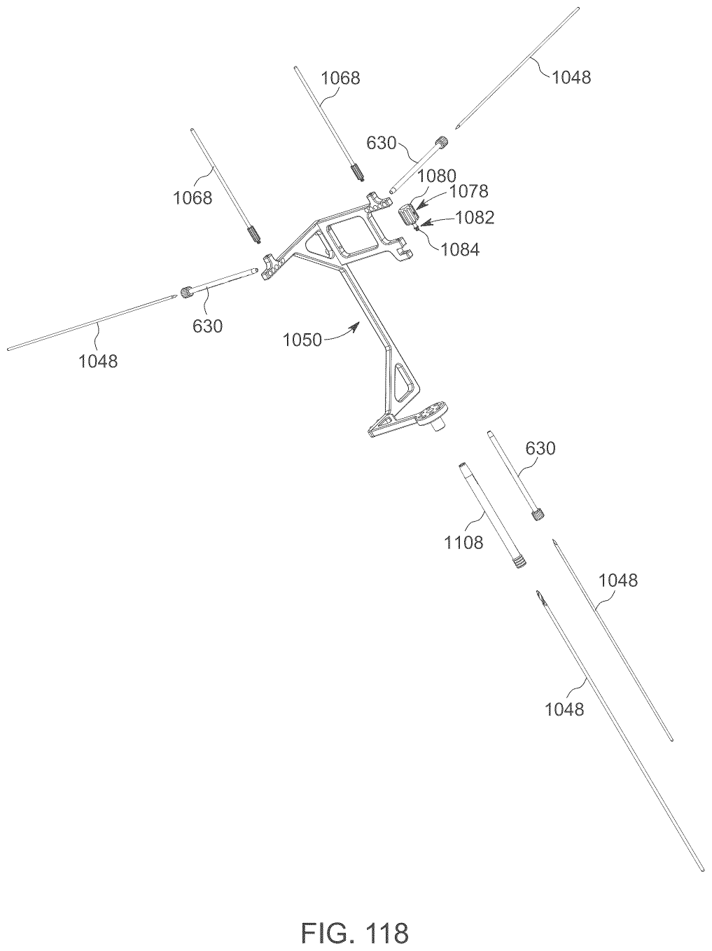

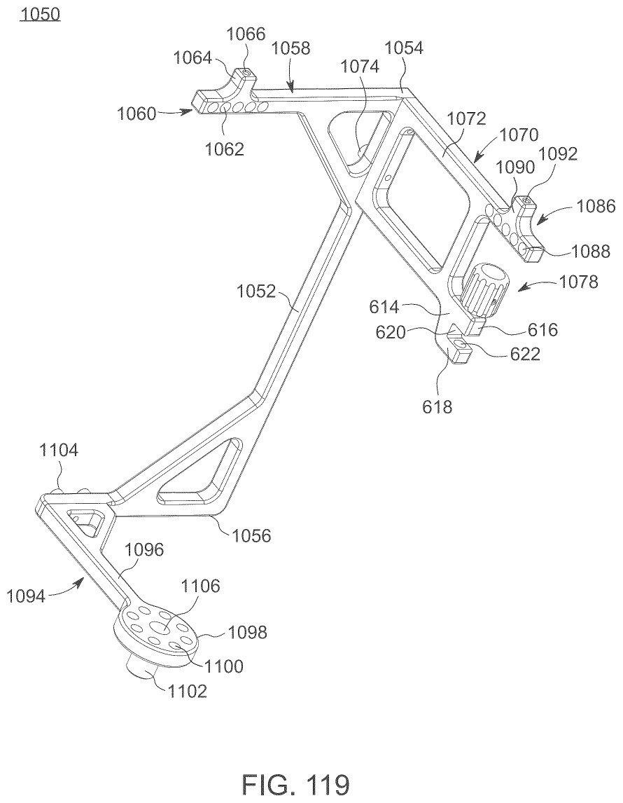

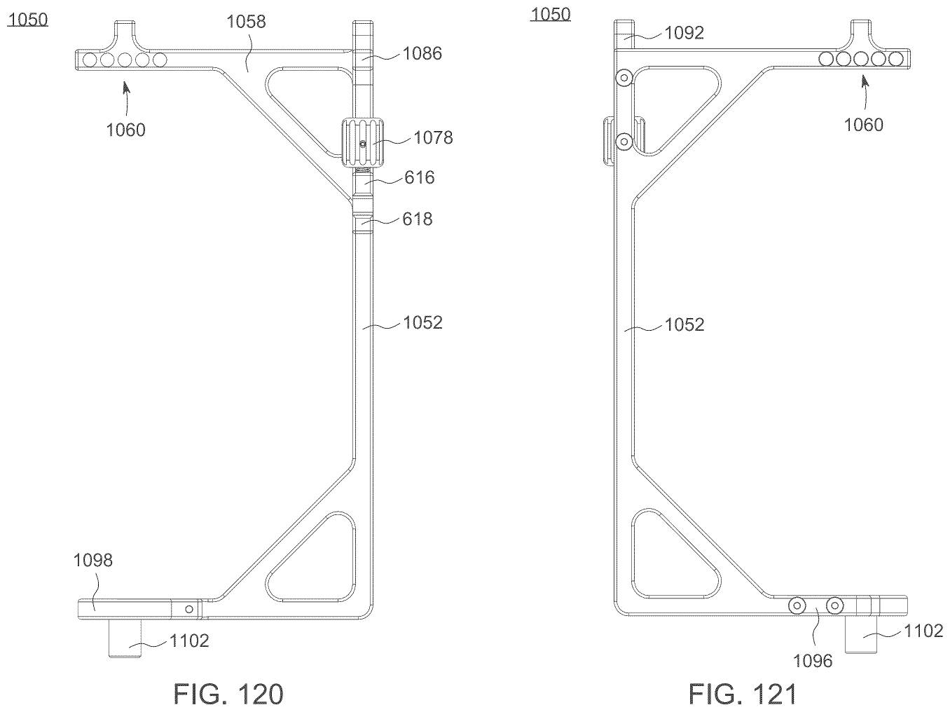

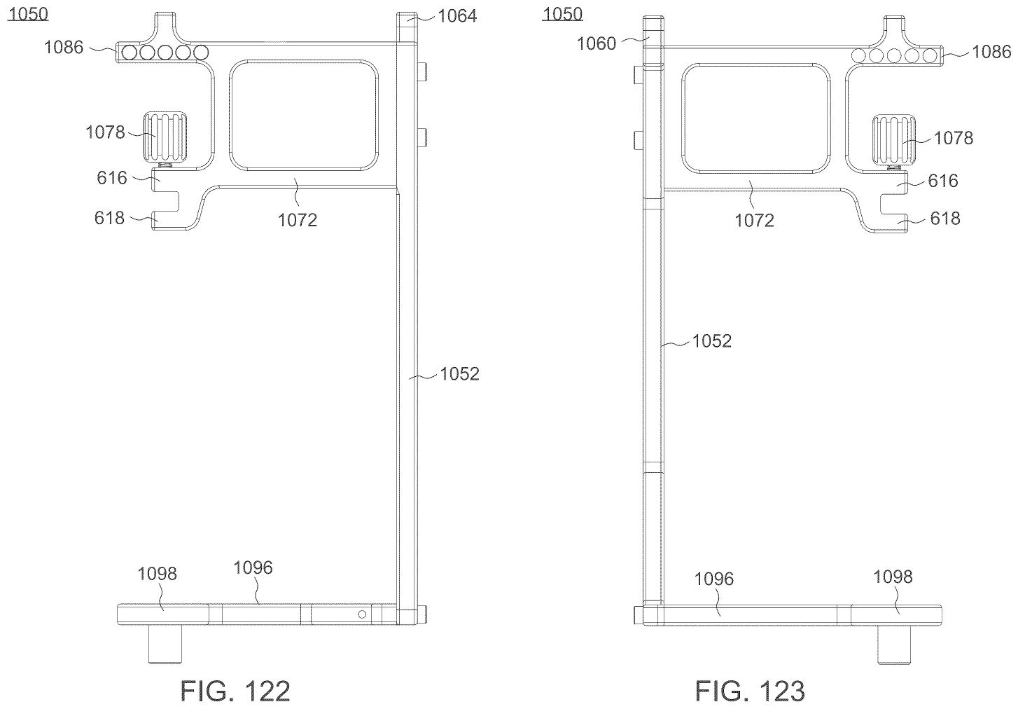

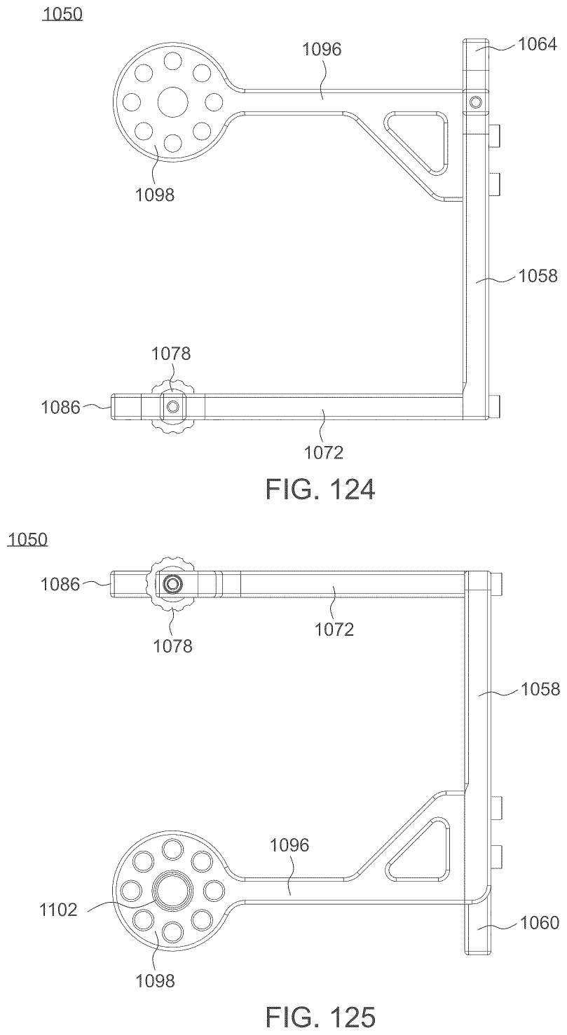

Referring now to FIGS. 115-125, a precision guide 1050 is shown. The guide 1050 includes a body 1052 with a first end 1054 and a second end 1056. The body 1052 may be, for example, shaped to have a main member with two extension extending from the first end 1054 and the second end 1056 in the same direction. The first end 1054 of the body 1052 includes a first alignment arm 1058 extending away from the body 1052 in a first direction. The alignment arm 1058 includes a free end or alignment end 1060 for receiving at least one k-wire 1048. The k-wire 1048 may be received, for example, through at least one of the plurality of through holes 1062 extending through the alignment end 1060. The through holes 1062 may be, for example, sized and shaped to receive a guide tube 630 for inserting a k-wire 1048 into the patient. The alignment end 1060 also includes a protrusion 1064 extending away from a top surface of the alignment end 1060. The protrusion 1064 may extend away from the arm 1058, for example, perpendicularly to the through holes 1062. The protrusion 1064 may include an opening 1066 extending from a first end toward the arm 1058. The opening 1066 may be, for example, threaded to receive an alignment member 1068 to assist with aligning the guide and implant during the surgical procedure.

The first end 1054 of the body 1052 may also include a coupling member 1070 with a body 1072, an attachment portion 614, and a second alignment end 1086, as shown in FIGS. 115-125. The body 1072 may have, for example, a rectangular shape with four members extending away from the body 1052. The attachment portion 614 may couple to and extend from the body 1072. The attachment portion 614 is as described in greater detail above and which will not be described again here for brevity's sake. The attachment portion 614 may include a through hole 622 extending through the legs 616, 618 and sized and shaped or configured to receive a coupling member 1078. The coupling member 1078 may include a head 1080 and a shaft 1082 extending from the head 1080. The shaft 1082 may be received within the through hole 622 and may include threaded portion 1084 to couple to the inserter 1030. The alignment end 1086 may include a plurality of through holes 1088 extending through the alignment end 1086. The through holes 1088 may be, for example, sized and shaped to receive a guide tube 630 for inserting a k-wire 1048 into the patient. The alignment end 1086 also includes a protrusion 1090 extending away from a top surface of the alignment end 1086. The protrusion 1090 may extend in a direction away from the top surface of the coupling member 1078, for example, perpendicularly to the through holes 1088. The protrusion 1090 may include an opening 1092 extending from a first end toward the plurality of through holes 1088. The opening 1092 may be, for example, threaded to receive an alignment member 1068 to assist with aligning the guide and implant during the surgical procedure.

With continued reference to FIGS. 115-125, the body 1052 may also include an alignment portion 1094 extending from the second end 1056 of the body 1052. The alignment portion 1094 may have a frame 1096 coupled to and extending from the body 1052. The frame 1096 may be coupled to the body 1052 with at least one fastener 1104. The frame 1096 may have an alignment hub 1098 coupled to the second end of the frame 1096. The alignment hub 1098 may have, for example, a round or circular shape. The alignment hub 1098 may include a plurality of through holes 1100 extending through the alignment hub 1098 from a top surface to a bottom surface. The through holes 1100 may be spaced circumferentially around the alignment hub 1098. The through holes 1100 may be, for example, sized and shaped to receive a guide tube 630 for inserting a k-wire 1048 into the patient. The alignment hub 1098 may also include a protrusion 1102 extending away from a bottom surface of the alignment hub 1098. The protrusion 1102 may be, for example, positioned at a center of the alignment hub 1098 and may include a through hole 1106 extending through the alignment hub 1098 and protrusion 1102. The through hole 1106 may be, for example, sized and shaped or configured to receive a drill guide 1108 for drilling an opening to receive an intramedullary nail.

After the patient's bones are prepared, a trial implant, such as, trial implant 1000 may be coupled to the inserter 1030 and inserted into the prepared bones, as shown in FIG. 105 to determine the proper size implant 100, 450, 700, 850 to be used. Imaging may be used to determine the position of the radiopaque markers 1020 in the implant trial 1000 while assessing the fit of the implant 100, 450, 700, 850. Once the implant 100, 450, 700, 850 is selected, it may be coupled to the inserter 1030 and inserted into the prepared bones. The implant 100, 450, 700, 850 may be, for example, implant 100, 450, 700, 850. Then, as shown in FIGS. 115-117, the precision guide 1050 may be coupled and used for insertion of a nail after the implant 100, 450, 700, 850 is inserted using an anterior approach.

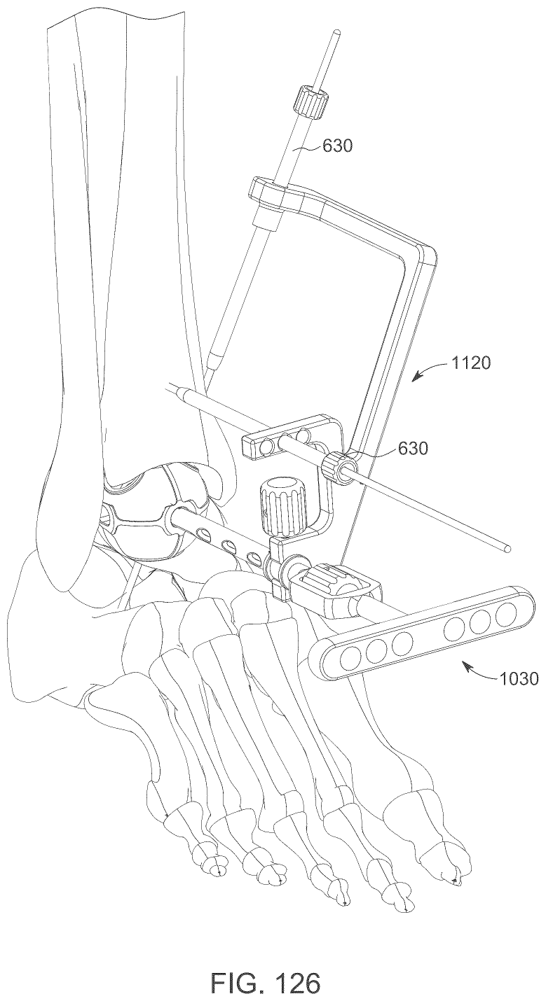

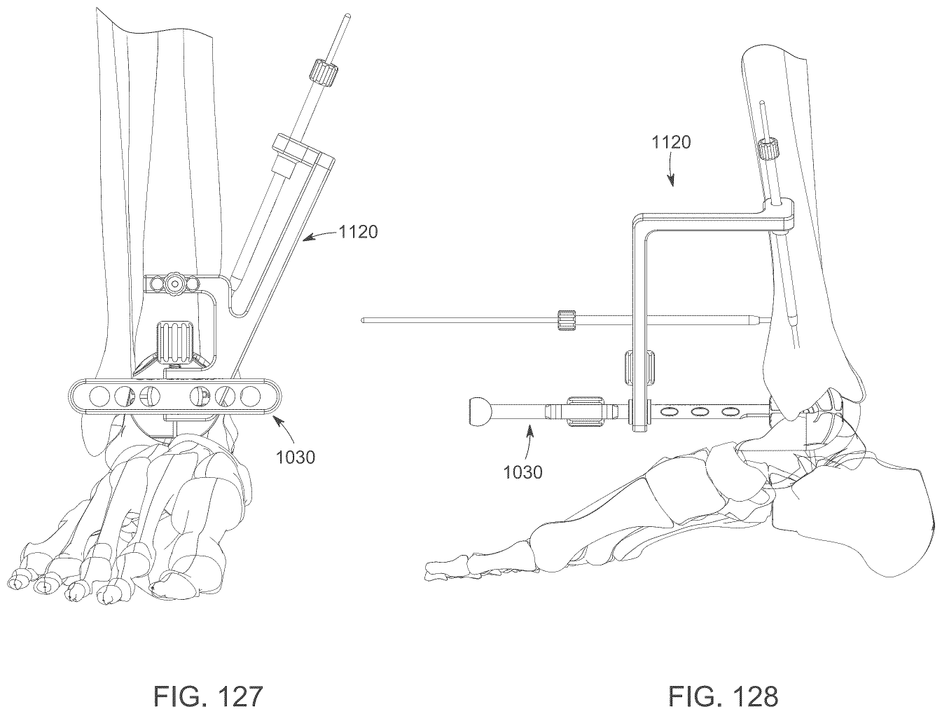

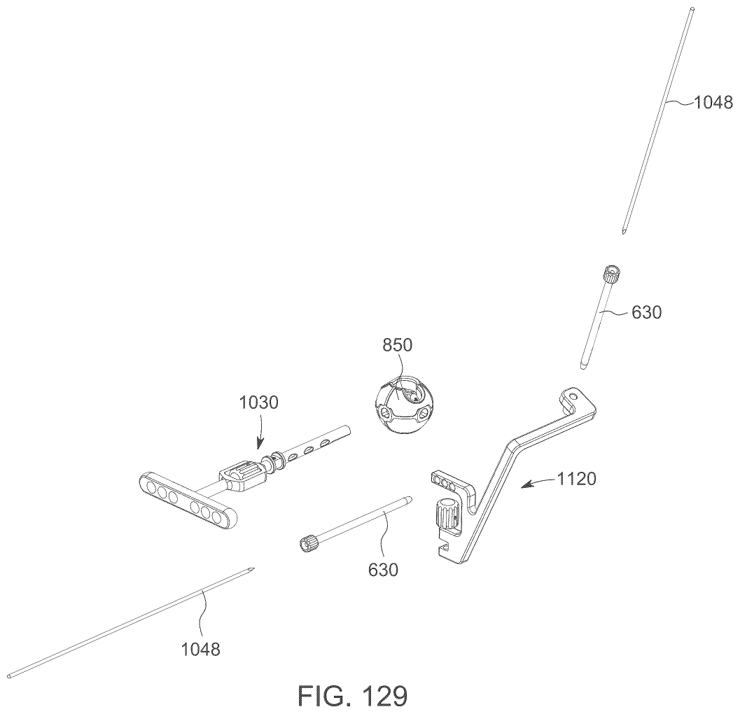

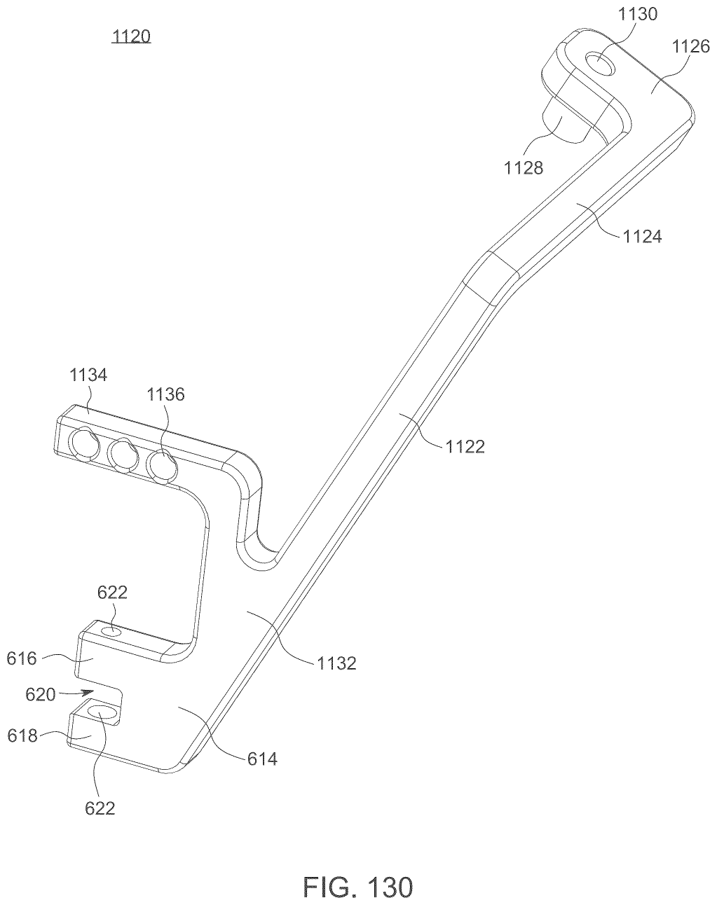

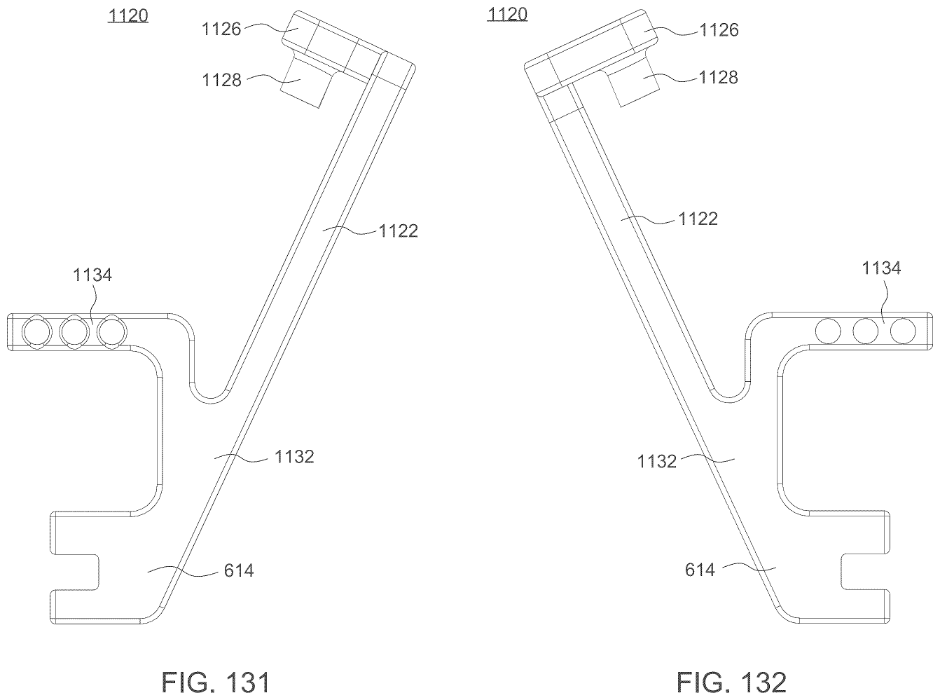

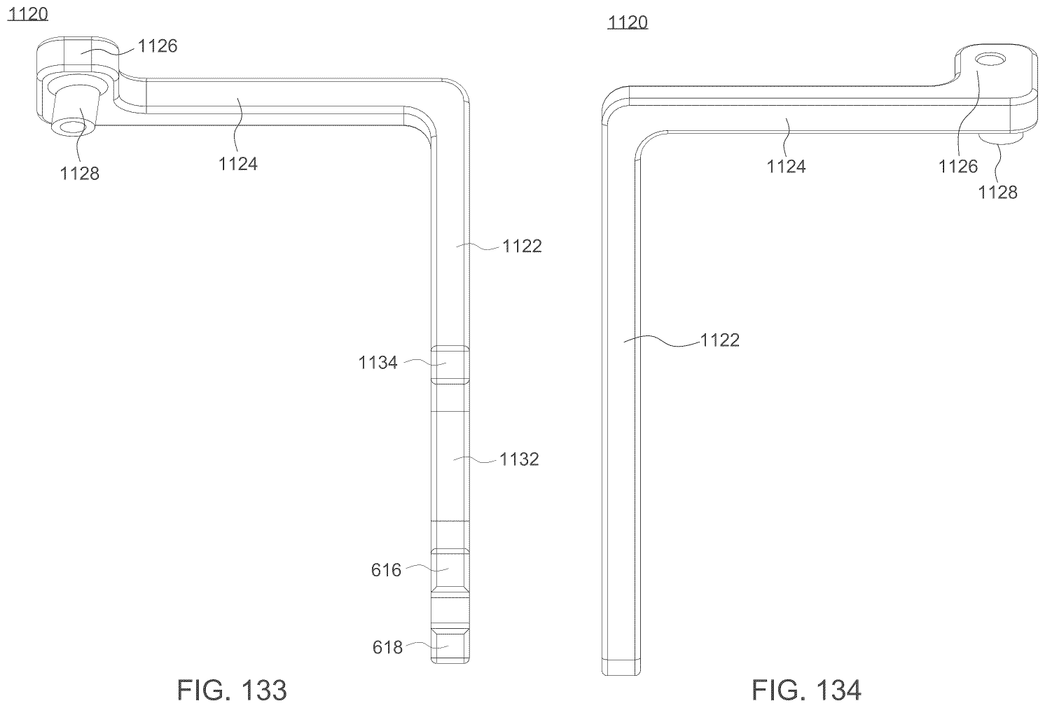

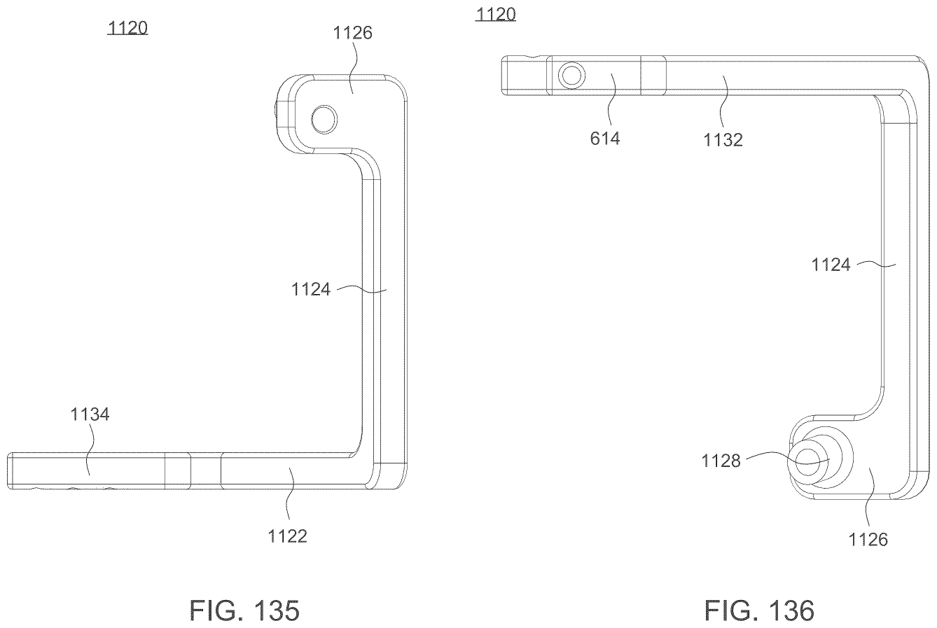

Referring now to FIGS. 126-136, another precision guide 1120 is shown. The guide 1120 includes a body 1122 with an arm 1124 extending away from a first end of the body 1122 in a first direction and a base 1132 positioned at the second end of the body 1122. The arm 1124 includes an alignment end 1126 extending from the arm 1124 at an end opposite the body 1122. The alignment end 1126 may, for example, extend away from the arm 1124 perpendicularly. The arm 1126 may include a protrusion 1128 extending away from a bottom surface of the alignment end 1126. The protrusion 1128 may include an opening 1130 extending through the protrusion 1128 and the alignment end 1126. The opening 1130 may be, for example, sized and shaped or configured to receive a guide tube 630 for inserting a k-wire into a patient.

The base 1132 of the guide 1120 may include an alignment member 1134 extending away from a first end of the base 1132 in a first direction. The alignment member 1134 may include a plurality of openings 1136 extending through the alignment member 1134 from a first side to a second side perpendicular to the length of the alignment member 1134. The opening 1136 may be, for example, sized and shaped or configured to receive a guide tube 630 for inserting a k-wire into a patient. The base 1132 may also include an attachment portion 614 extending away from a second end of the base 1132 also in the first direction. The alignment member 1134 is positioned superior to the attachment portion 614. The attachment portion 614 is as discussed in greater detail above and will not be described again here for brevity's sake. The attachment portion 614 is configured to couple the guide 1120 to the insertion instrument 1030.

After the patient's bones are prepared, a trial implant, such as, trial implant 1000 may be coupled to the inserter 1030 and inserted into the prepared bones, as shown in FIG. 105 to determine the proper size implant 100, 450, 700, 850 to be used. Imaging may be used to determine the position of the radiopaque markers 1020 in the implant trial 1000 while assessing the fit of the implant 100, 450, 700, 850. Once the implant 100, 450, 700, 850 is selected, it may be coupled to the inserter 1030 and inserted into the prepared bones. The implant 100, 450, 700, 850 may be, for example, implant 100, 450, 700, 850. Then, as shown in FIGS. 126-128, the precision guide 100, 450, 700, 850 may be coupled and used for insertion of a crossing screw after the implant 100, 450, 700, 850 is inserted using an anterior approach.

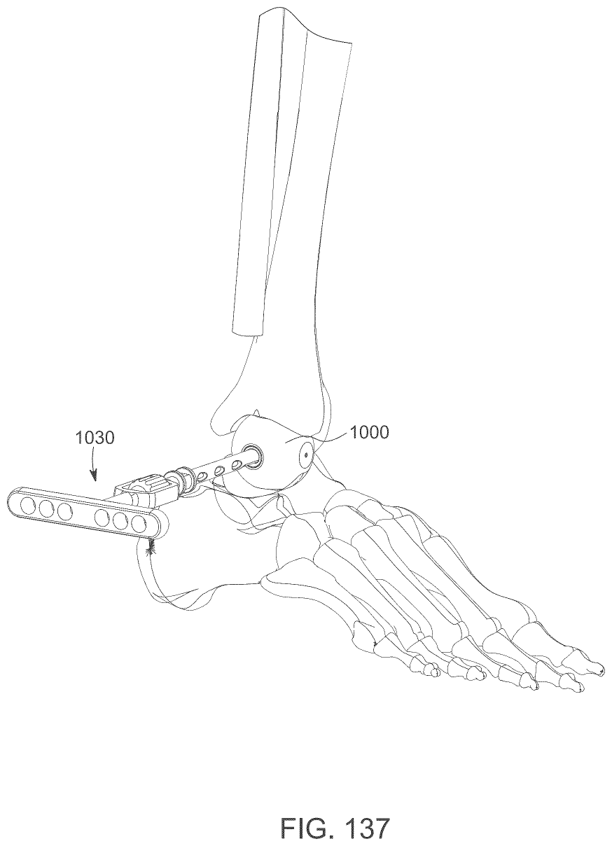

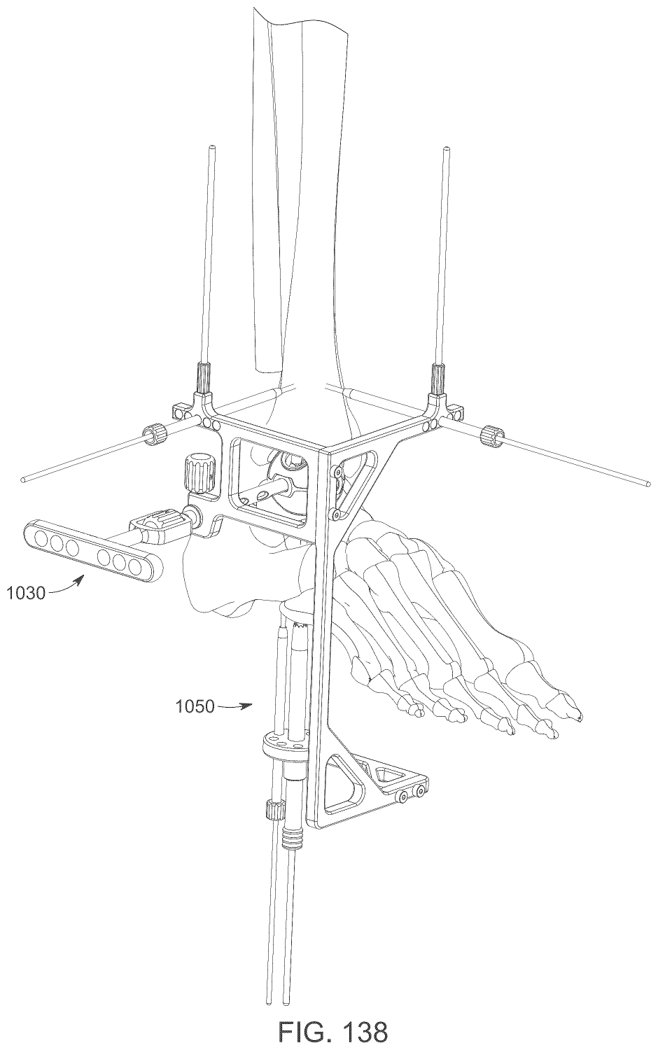

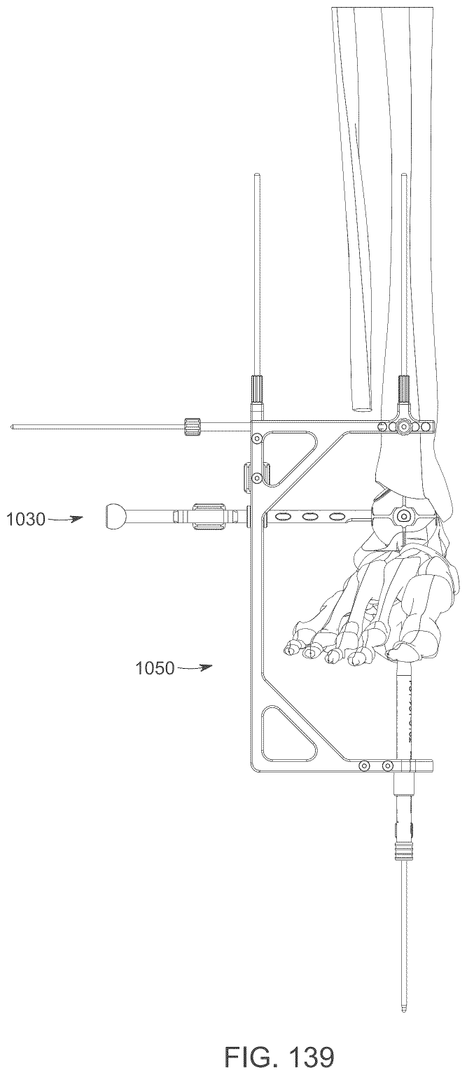

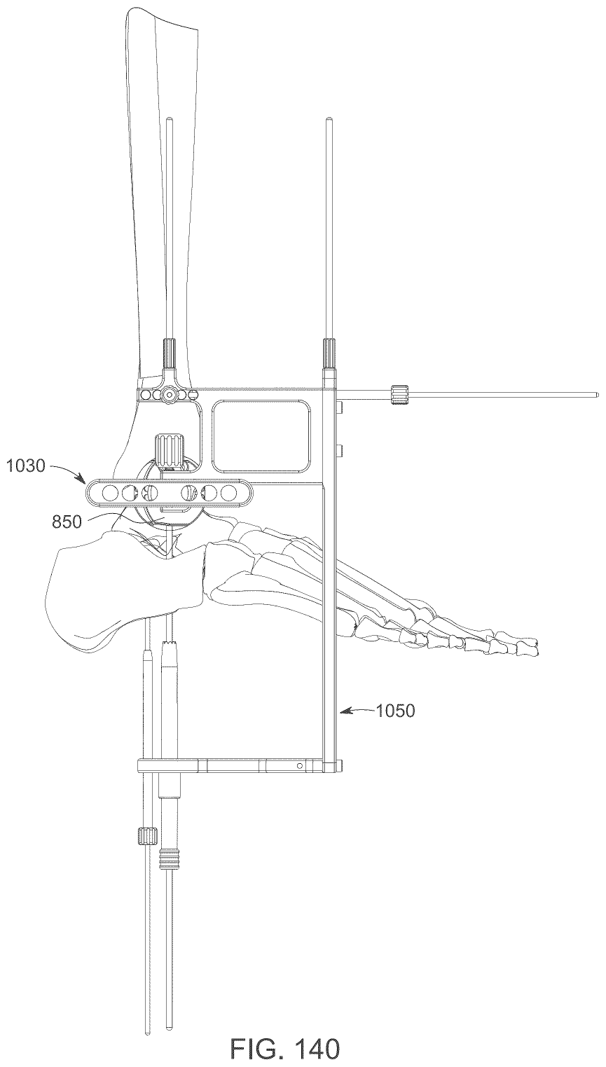

Referring now to FIGS. 137-140, the patient's bones are prepared, a trial implant, such as, trial implant 1000 may be coupled to the inserter 1030 and inserted into the prepared bones, as shown in FIG. 137 to determine the proper size implant 100, 450, 700, 850 to be used. Imaging may be used to determine the position of the radiopaque markers 1020 in the implant trial 1000 while assessing the fit of the implant 100, 450, 700, 850. Once the implant 100, 450, 700, 850 is selected, it may be coupled to the inserter 1030 and inserted into the prepared bones. The implant 100, 450, 700, 850 may be, for example, implant 100, 450, 700, 850. Then, as shown in FIGS. 138-140, the precision guide 1050 may be coupled and used for insertion of a nail after the implant 100, 450, 700, 850 is inserted using a lateral approach.

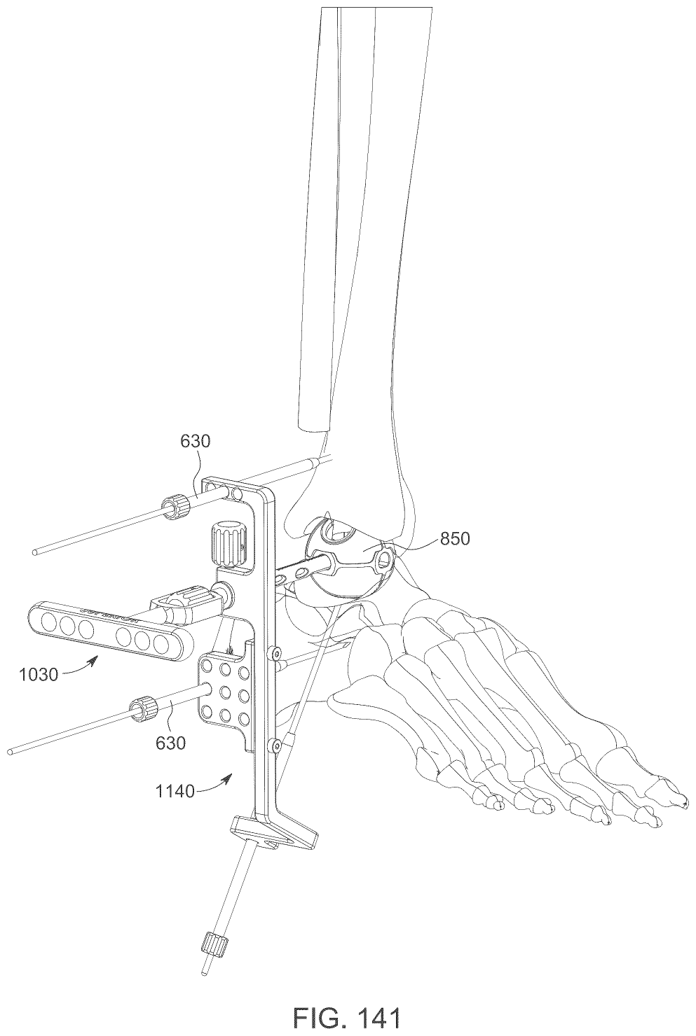

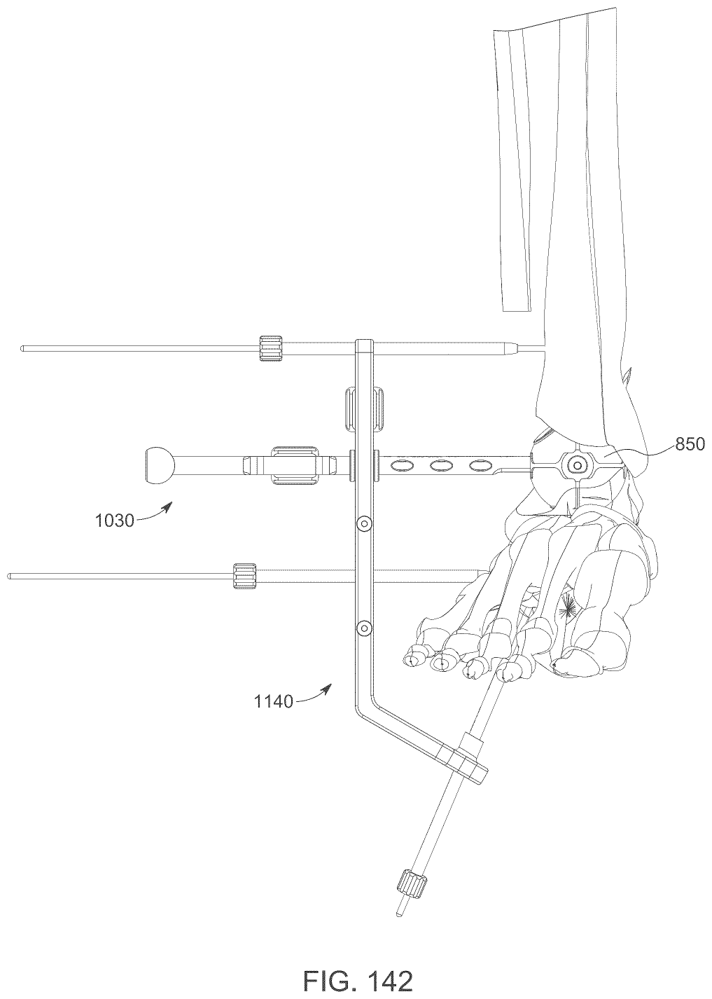

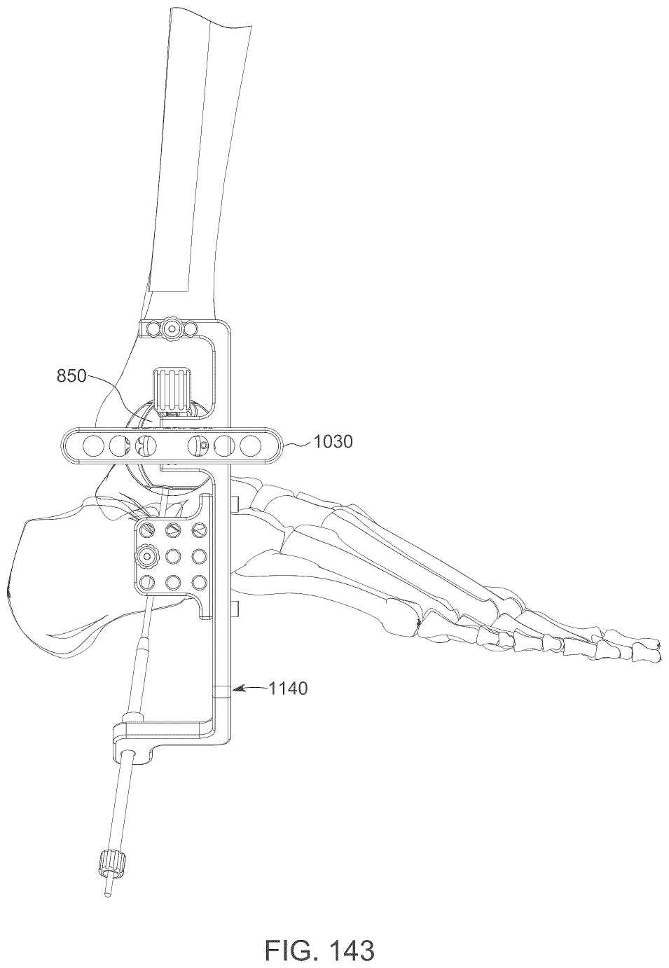

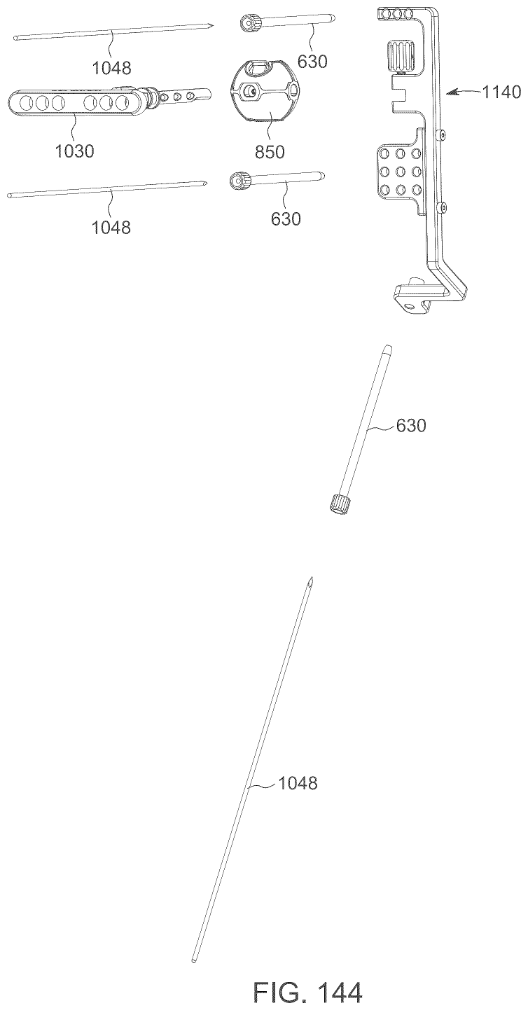

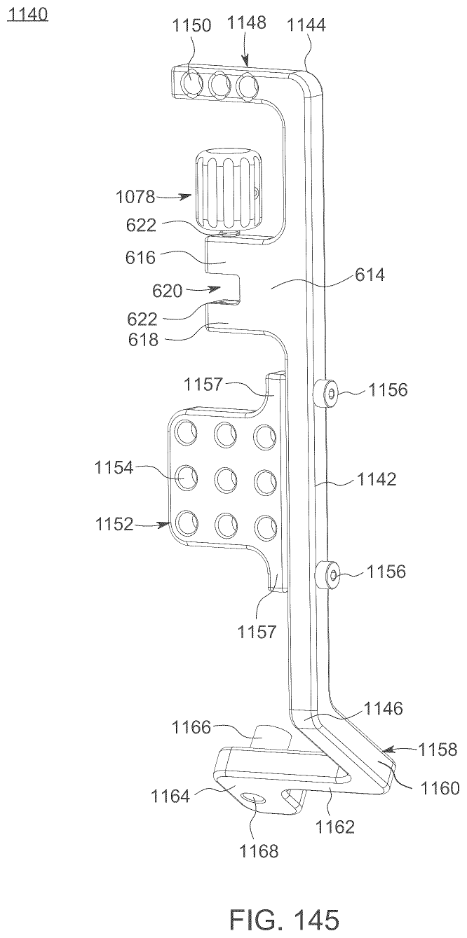

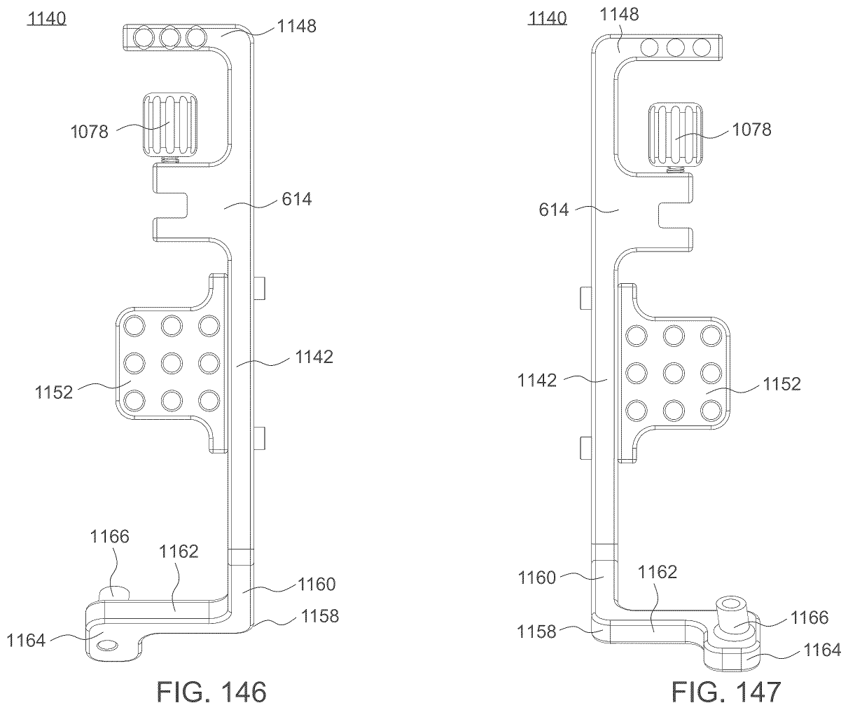

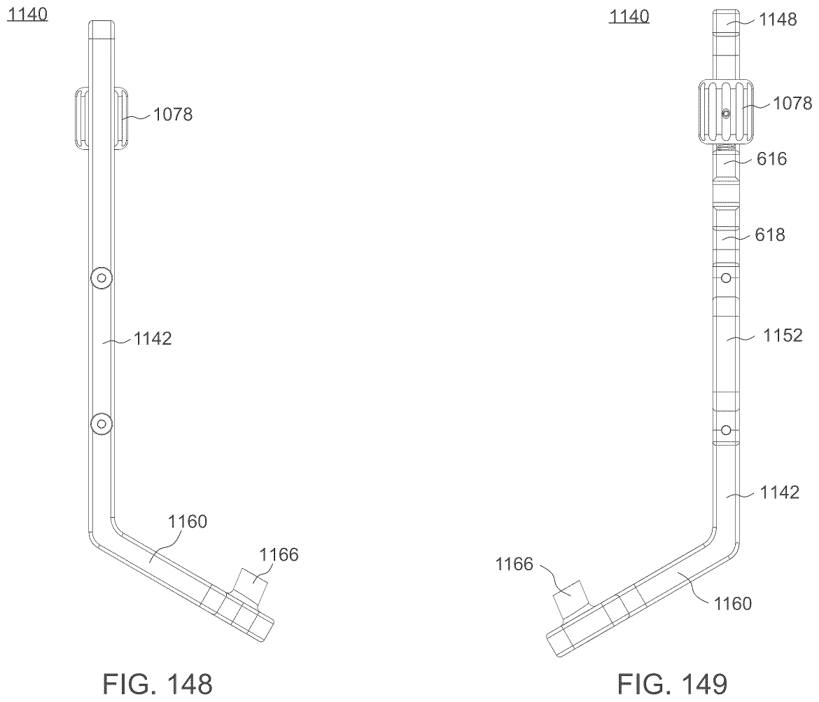

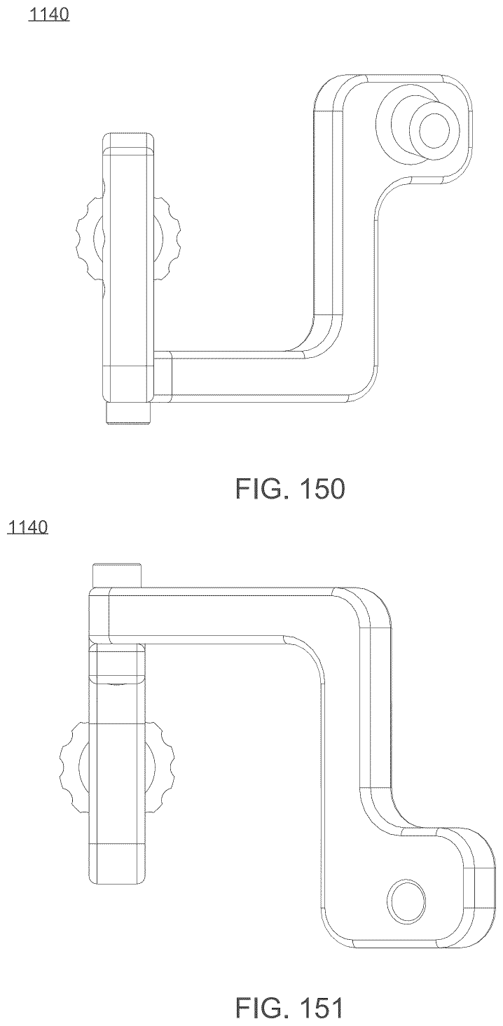

Referring now to FIGS. 141-151, another precision guide 1140 is shown. The guide 1140 includes a body 1142 with a first end 1144 and a second end 1146. The guide 1140 includes a first alignment member 1148 positioned at and extending from the first end 1144. The first alignment member 1148 extends from the body 1142 perpendicularly. The first alignment member 1148 includes a plurality of openings 1150 extending from a first side to a second side of the first alignment member 1148. The plurality of openings 1150 may be, for example, three openings although alternative numbers of openings 1150 are also contemplated. The openings 1150 may be, for example, sized and shaped or configured to receive a guide tube 630 for inserting a k-wire into a patient. The guide 1140 also includes an attachment portion 614 for coupling to the insertion instrument 1030. The attachment portion 614 is coupled to and extends from the body 1142 between the first end 1144 and the second end 1146. The attachment portion 614 is as described in greater detail above and which will not be described again here for brevity's sake. The guide 1140 also includes a second alignment member 1152 coupled to and extend from the body 1142. The second alignment member 1152 is coupled to the body 1142 with fasteners 1156. The fasteners 1156 engage tabs 1157 extending from a side of the second alignment member 1152. The second alignment member 1152 includes a plurality of openings 1154 extending through the second alignment member 1152 from a first side to a second side. The second alignment member 1152 may have, for example, a rectangular shape and may have the openings 1154 evenly spaced across the member 1152. The second alignment member 1152 is positioned between the attachment portion 614 and the second end 1146 of the body 1142. The openings 1154 may be, for example, sized and shaped or configured to receive a guide tube 630 for inserting a k-wire into a patient.

With continued reference to FIGS. 141-151, the guide 1140 also includes a lower member 1158 extending from the second end 1146 of the body 1142. The lower member 1158 includes a first leg 1160 extending from the body 1142 in a direction perpendicular to the direction of the first and second alignment members 1148, 1152 and the attachment portion 614. The first leg 1160 extends from the body 1142 at an angle away from the body 1142. The lower member 1158 also has a second leg 1162 extending away from a second end of the first leg 1160. The second leg 1162 extends from the first leg 1160 perpendicularly and in the direction of the first and second alignment members 1148, 1152 and the attachment portion 614 to position the alignment end 1164 superior to the members 1148, 1152 and attachment portion 614. The alignment end 1164 is coupled to the second end of the second leg 1162. The alignment end 1164 may, for example, extend away from the second leg 1162 perpendicularly. The alignment end 1164 may include a protrusion 1166 extending away from a bottom surface of the alignment end 1164. The protrusion 1166 may include a through hole 1168 extending through the protrusion 1166 and the alignment end 1164. The through hole 1168 may be, for example, sized and shaped or configured to receive a guide tube 630 for inserting a k-wire into a patient.

Referring now to FIGS. 137 and 141-143, the patient's bones are prepared, a trial implant, such as, trial implant 1000 may be coupled to the inserter 1030 and inserted into the prepared bones, as shown in FIG. 137 to determine the proper size implant 100, 450, 700, 850 to be used. Imaging may be used to determine the position of the radiopaque markers 1020 in the implant trial 1000 while assessing the fit of the implant 100, 450, 700, 850. Once the implant 100, 450, 700, 850 is selected, it may be coupled to the inserter 1030 and inserted into the prepared bones. The implant 100, 450, 700, 850 may be, for example, implant 100, 450, 700, 850. Then, as shown in FIGS. 141-143, the precision guide 100, 450, 700, 850 may be coupled and used for insertion of a crossing screw after the implant 100, 450, 700, 850 is inserted using a lateral approach.

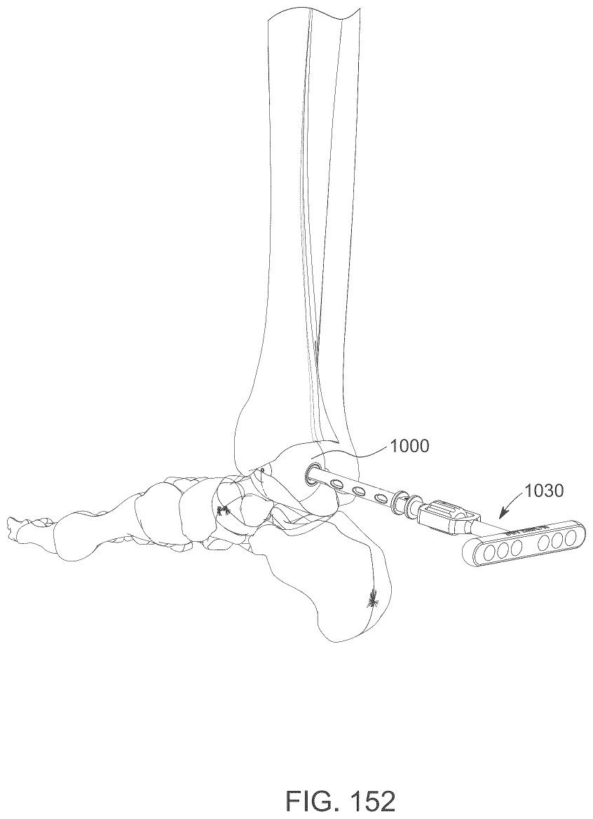

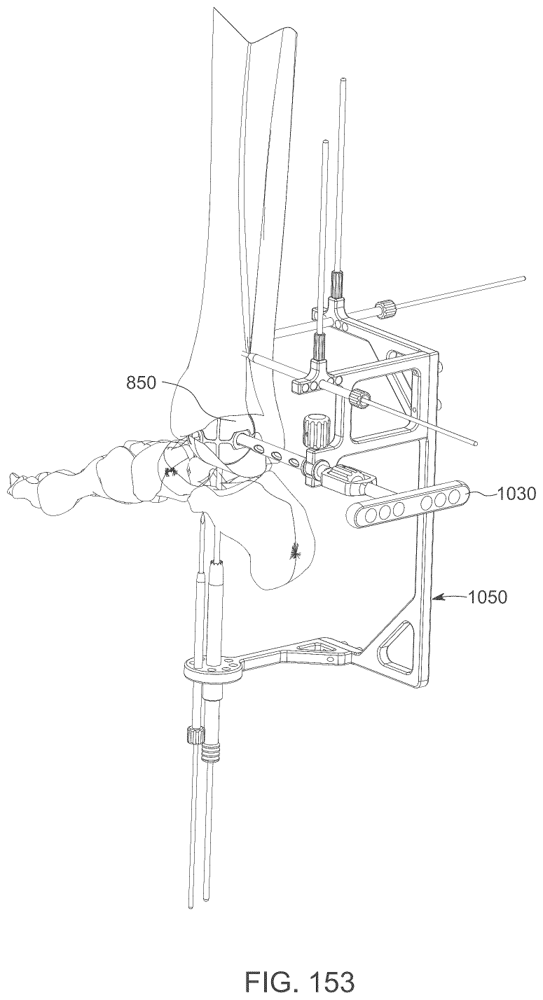

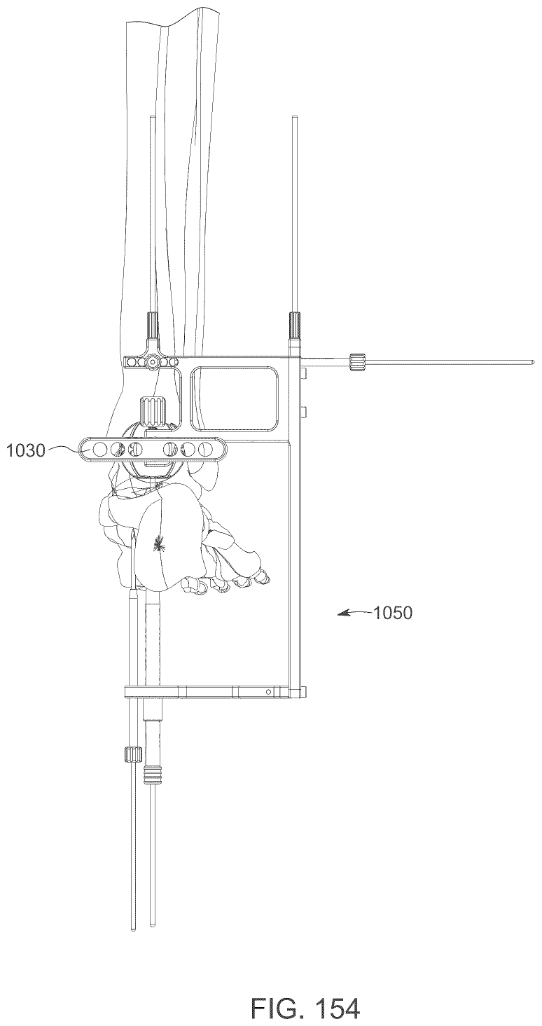

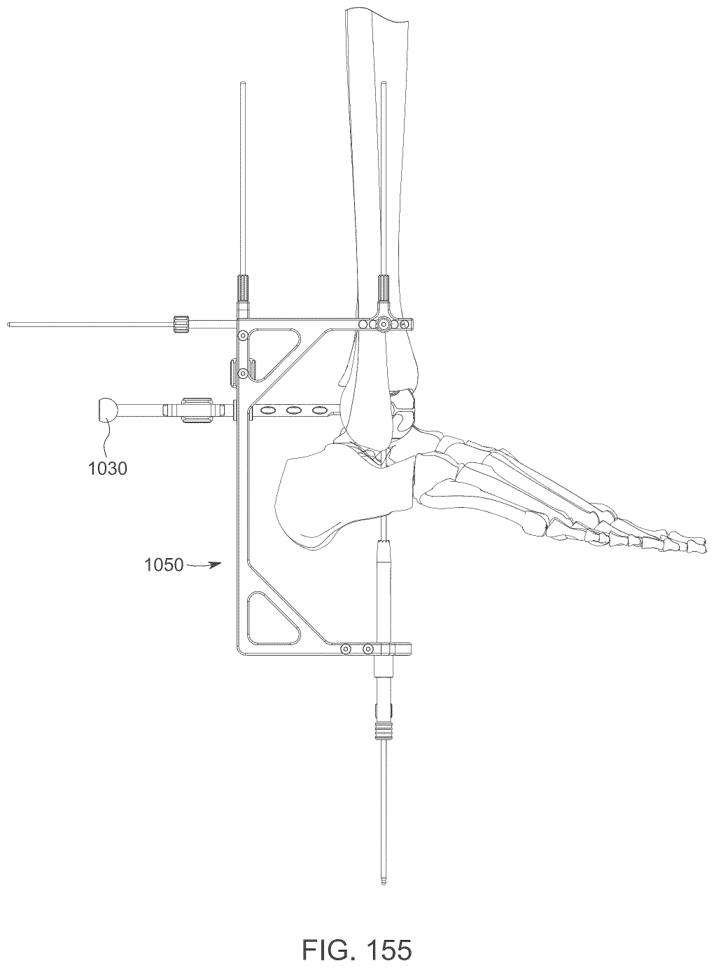

Referring now to FIGS. 152-155, the patient's bones are prepared, a trial implant, such as, trial implant 1000 may be coupled to the inserter 1030 and inserted into the prepared bones, as shown in FIG. 152 to determine the proper size implant 100, 450, 700, 850 to be used. Imaging may be used to determine the position of the radiopaque markers 1020 in the implant trial 1000 while assessing the fit of the implant 100, 450, 700, 850. Once the implant 100, 450, 700, 850 is selected, it may be coupled to the inserter 1030 and inserted into the prepared bones. The implant 100, 450, 700, 850 may be, for example, implant 100, 450, 700, 850. Then, as shown in FIGS. 153-155, the precision guide 1050 may be coupled and used for insertion of a nail after the implant 100, 450, 700, 850 is inserted using a posterior approach.

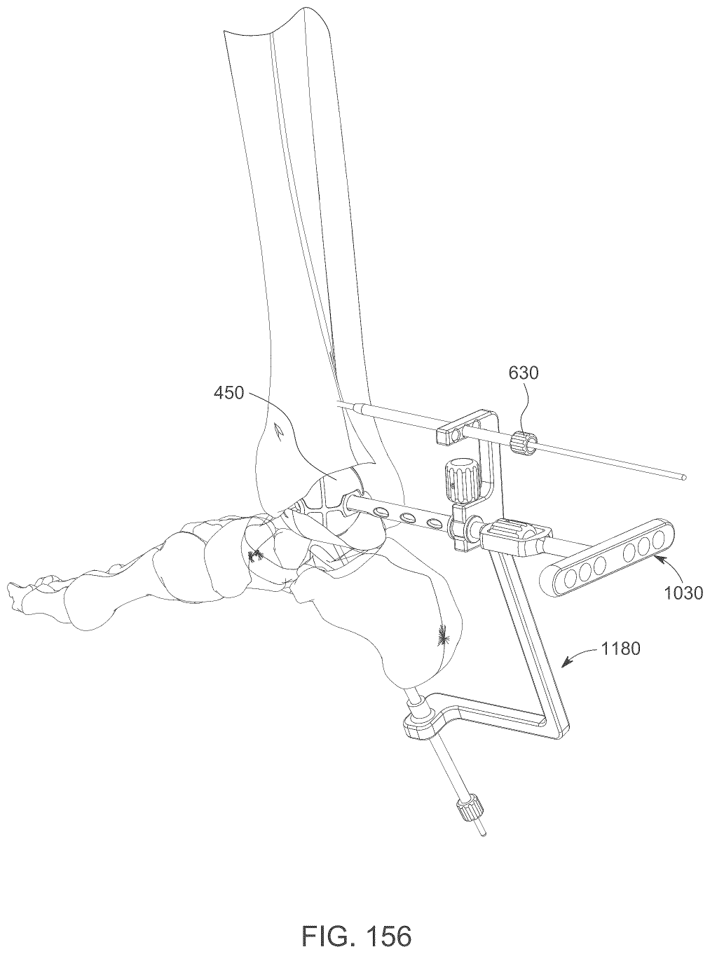

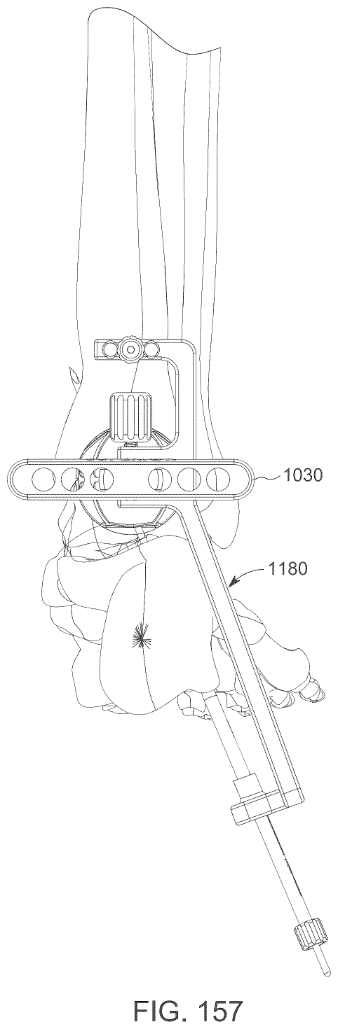

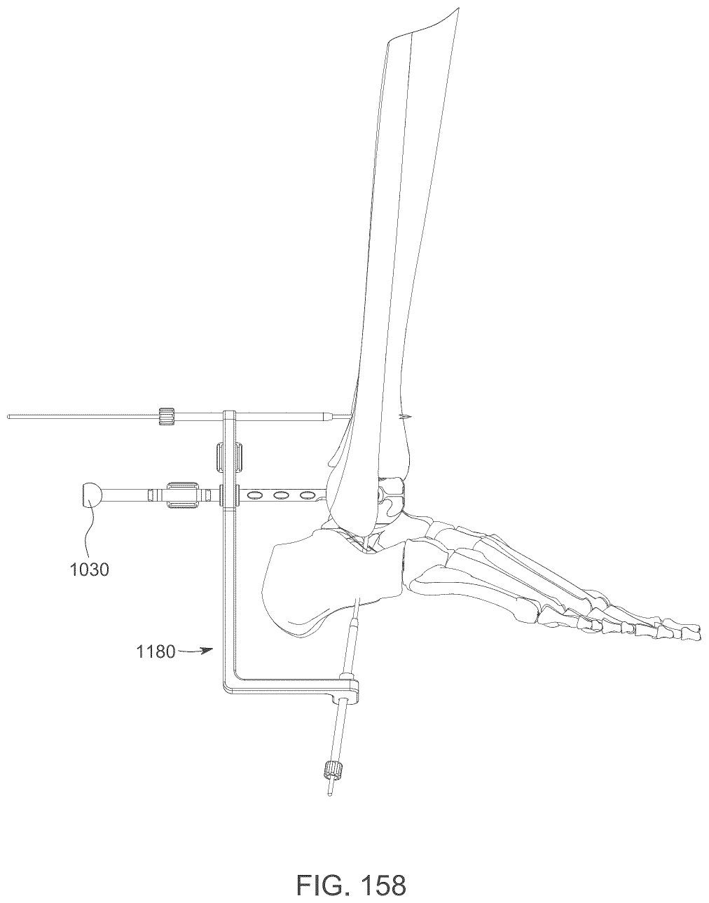

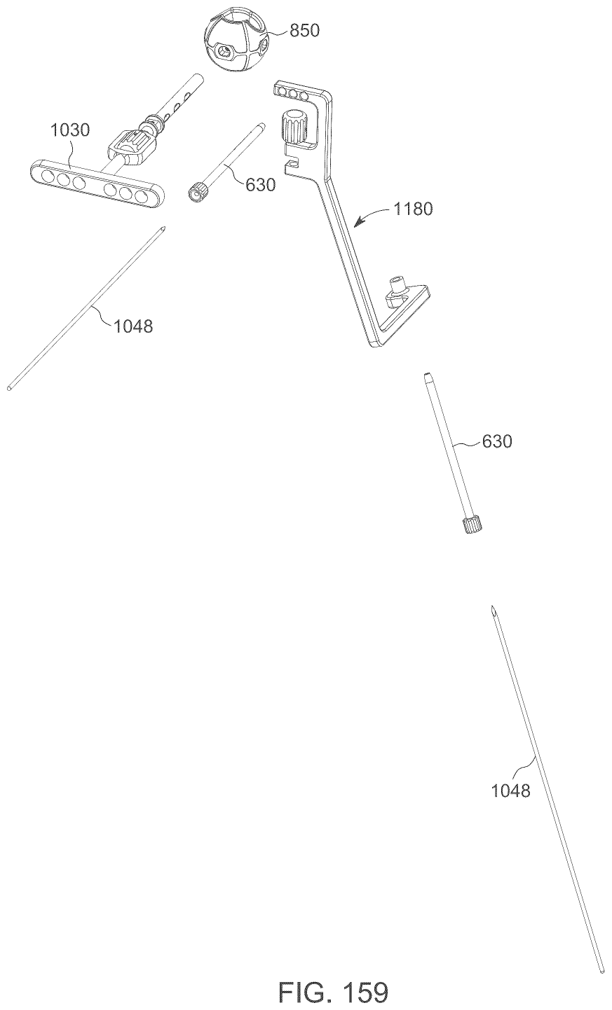

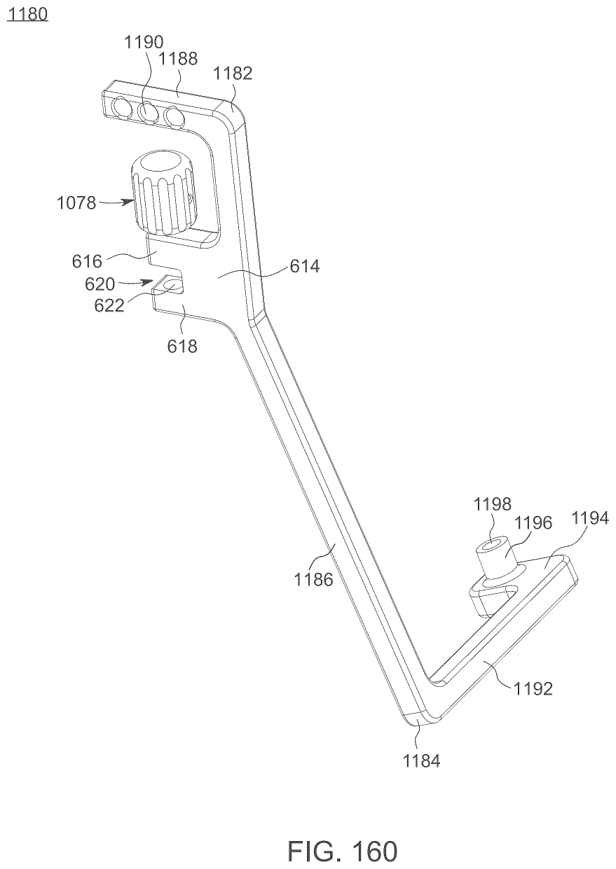

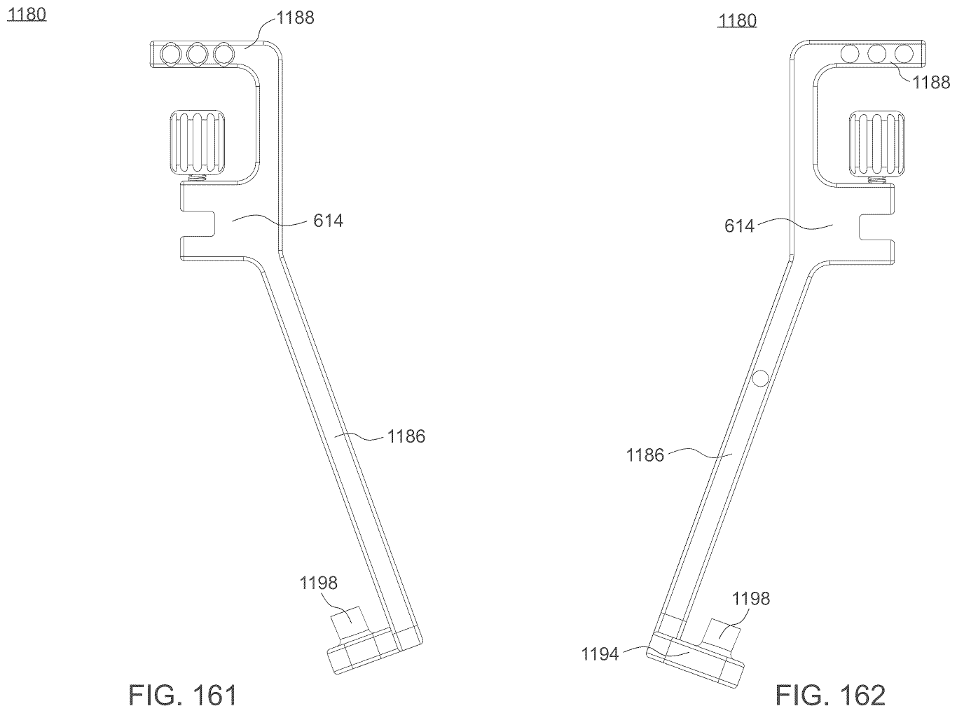

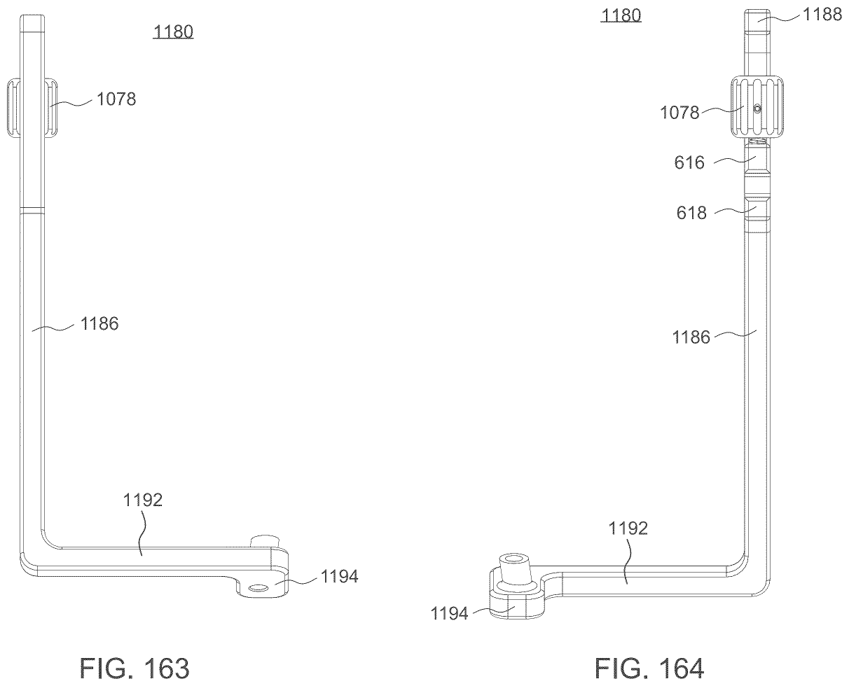

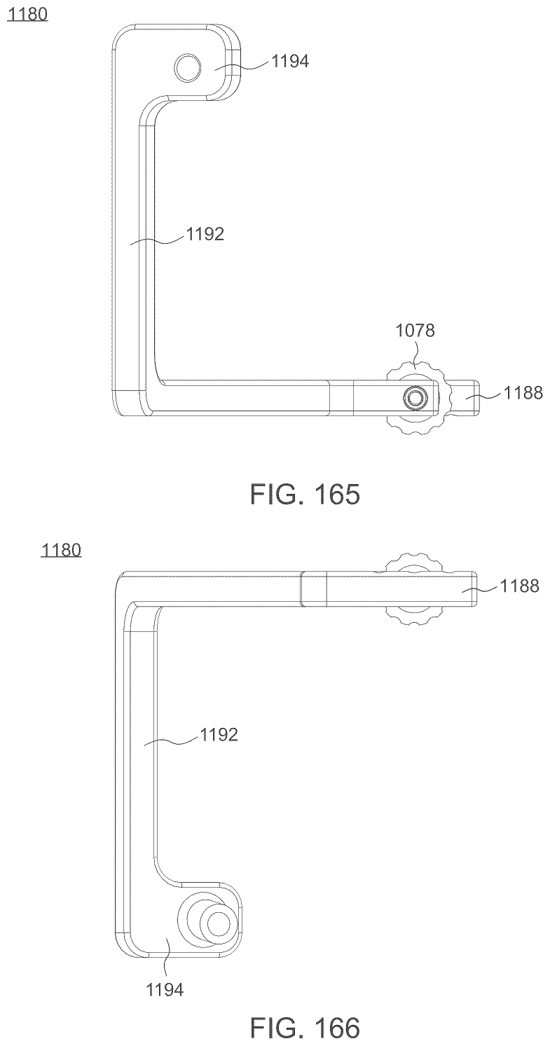

Referring now to FIGS. 156-166, another precision guide 1180 is shown. The guide 1180 includes a body 1186 with a first end 1182 and a second end 1184. The guide 1180 also includes an alignment member 1188 coupled to and extending from the first end 1182 of the body 1186. The alignment member 1188 extends in a first direction perpendicular to a portion of the body 1186. The guide 1180 also includes an attachment portion 614 extending from the body 1186. The attachment portion 614 extends from the body 1186 between the alignment member 1188 and the second end 1184. The attachment portion 614 may be as described above with reference to guide 550 and which will not be described again here for brevity's sake.

The guide 1180 also includes an arm 1192 extending away from the second end 1184 of the body 1186 in a second direction perpendicular to the body 1186, the alignment member 1188 and the attachment portion 614. The arm 1192 includes an alignment end 1194 extending from the arm 1192 at an end opposite the body 1186. The alignment end 1194 may, for example, extend away from the arm 1192 perpendicularly. The alignment end 1194 may include a protrusion 1196 extending away from a top surface of the alignment end 1194. The protrusion 1196 may include a through hole 1198 extending through the protrusion 1196 and the alignment end 1194. The through hole 1198 may be, for example, sized and shaped or configured to receive a guide tube 630 for inserting a k-wire into a patient.

The base 1132 of the guide 1120 may include an alignment member 1134 extending away from a first end of the base 1132 in a first direction. The alignment member 1134 may include a plurality of openings 1136 extending through the alignment member 1134 from a first side to a second side perpendicular to the length of the alignment member 1134. The opening 1136 may be, for example, sized and shaped or configured to receive a guide tube 630 for inserting a k-wire into a patient. The base 1132 may also include an attachment portion 614 extending away from a second end of the base 1132 also in the first direction. The alignment member 1134 is positioned superior to the attachment portion 614. The attachment portion 614 is as discussed in greater detail above and will not be described again here for brevity's sake. The attachment portion 614 is configured to couple the guide 1120 to the insertion instrument 1030.

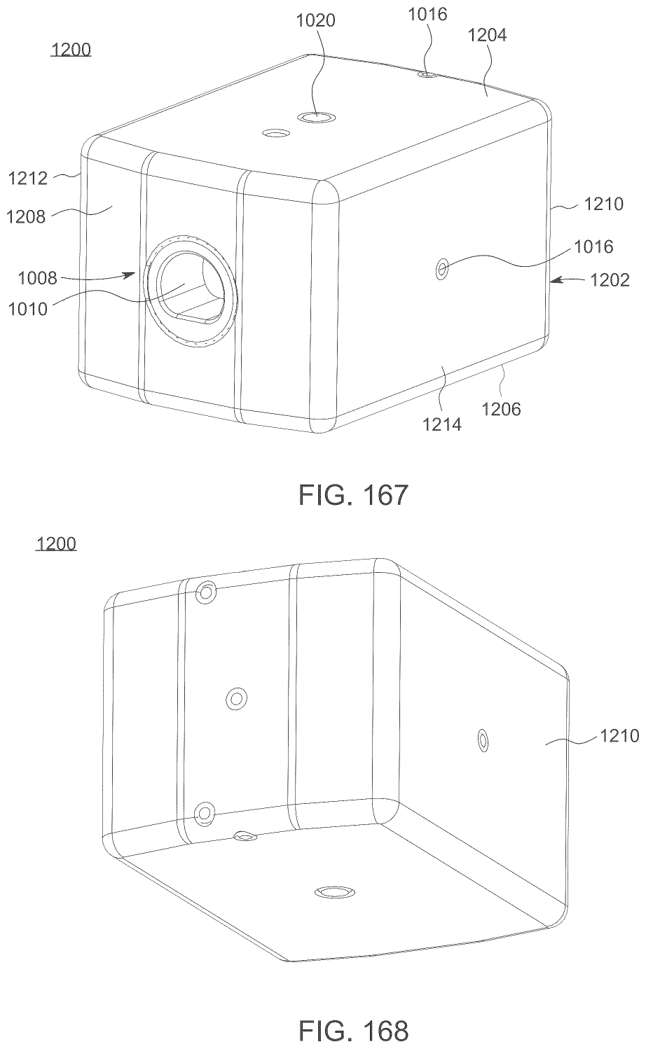

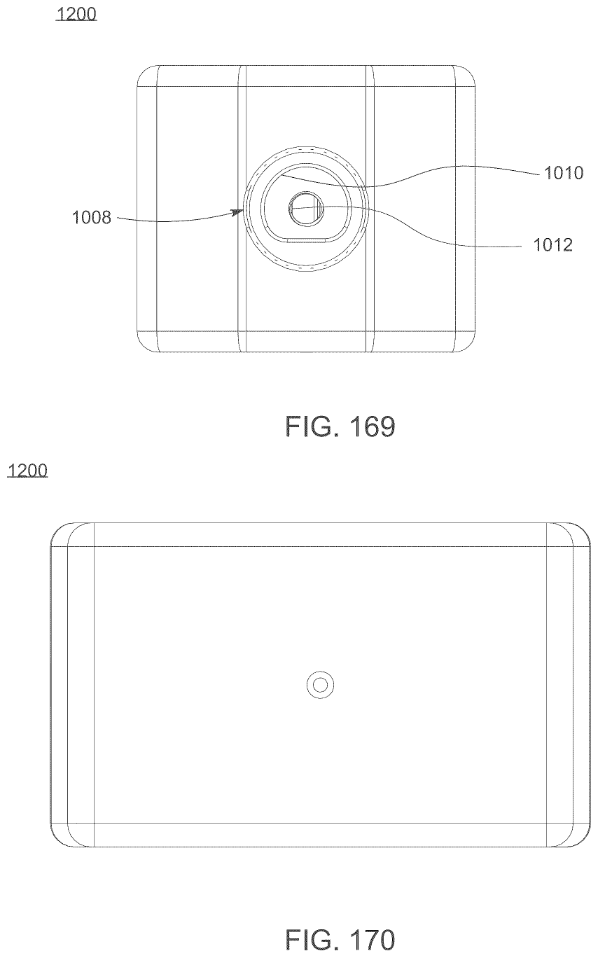

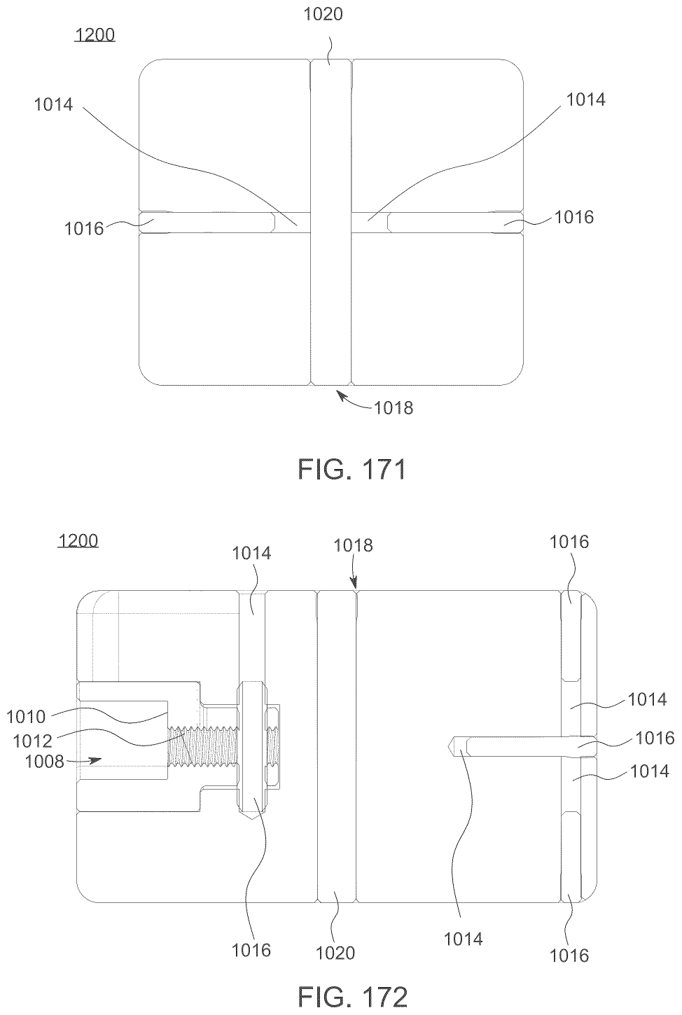

Referring now to FIGS. 167-172, another implant trial 1200 is shown. The implant trial 1200 includes an exterior surface 1202 having a rectangular shape, a top surface 1204, a bottom surface 1206, a front 1208, a back 1210, a first side 1212, and a second side 1214. The implant trial 1200 includes an inserter opening 1008 inset into the exterior surface 1202 of the trial 1200. The inserter opening 1008 includes a recessed region 1010 extending from the exterior surface 1202 into the trial 1200 and a through hole 1012 extending from the interior surface of the recessed region 1010 into the trial 1200. The through hole 1012 may be, for example, threaded to engage and couple to a threaded portion of an inserter 1030. The recessed region 1010 may have, for example, a size that is larger than the size of the through hole 1012 to receive an end of an inserter 1030. The trial 1200 may also include a plurality of holes 1014 for receiving radiopaque markers 1016.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

As may be recognized by those of ordinary skill in the art based on the teachings herein, numerous changes and modifications may be made to the above-described and other embodiments of the present disclosure without departing from the scope of the disclosure. The components of the instruments, implants, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the instruments, implants, and/or systems may include more or fewer components or features than the embodiments as described and illustrated herein. Accordingly, this detailed description of the currently-preferred embodiments is to be taken in an illustrative, as opposed to limiting of the disclosure.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers, and/or sections, these elements, components, regions, layers, and/or sections should not be limited by these terms. These terms are only used to distinguish one element, component, region, layer, and/or section from another element, component, region, layer, and/or section. Thus, a first element, component, region, layer, and/or section discussed below could be termed a second element, component, region, layer, and/or section without departing from the teachings of the present disclosure.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The transition term “comprising” encompasses the transition terms “consisting of” and “consisting essentially of.” The term and/or is used herein to mean both “and” as well as “or.” For example, “A and/or B” is construed to mean A, B or A and B.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Various numerical ranges are disclosed herein. These ranges are inclusive of the endpoints as well as numerical values between these endpoints. The numbers in these ranges are interchangeable.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

Having thus described the preferred embodiments, the invention is now claimed to be:

1. An implant, comprising:
an interior frame;
an external frame surrounding the interior frame; and
a lattice structure extending between at least a portion of the interior frame and the external frame.

2. The implant of claim 1, wherein the interior frame comprises:
at least one first intermediate portion positioned at a top side of the implant; and a second intermediate portion positioned at a bottom side of the implant,
wherein the at least one first intermediate portion is not directly coupled to the second intermediate portion, and wherein the at least one first intermediate portion is coupled to the external frame on the top side of the implant and the second intermediate portion is coupled to the external frame on the bottom side of the implant.

3. The implant of claim 2, wherein the at least one first intermediate portion comprises:
at least one first segment extending between a first side and a second side; and at least one second segment extending between the first side and the second side spaced apart from the at least one first segment a first distance,
wherein a first end of the at least one first segment is coupled to a first end of the at least one second segment by a first guide hole member, and
wherein a second end of the at least one first segment is coupled to a second end of the at least one second segment by a second guide hole member.

4. The implant of claim 3, wherein the first guide hole member is curved from the top side toward the bottom side,
wherein the second guide hole member is curved from the top side toward the bottom side, and
wherein a first trajectory extends through the first guide hole member at the top side, the implant, and the second intermediate portion at the bottom side, and wherein a second trajectory extends through the second guide hole member at the top side, the implant, and the second intermediate portion at the bottom side.

5. The implant of claim 3, wherein the second intermediate portion has an oval shape with a length longer than the first distance between the at least one first segment and the at least one second segment of the at least one first intermediate portion.

6. The implant of claim 3, wherein the at least one first intermediate portion and the second intermediate portion are coupled to the external frame by the lattice structure.

7. The implant of claim 3, wherein the at least one first intermediate portion comprises:
a first lobe positioned on a first side of the implant;
a second lobe positioned on a second side of the implant;
a first connecting segment with a first end and a second end, wherein the first end of the first connecting segment is coupled to a first end of the first lobe, and wherein the second end of the first connecting segment is coupled to a first end of the second lobe; and
a second connecting segment with a first end and a second end, wherein the first end of the second connecting segment is coupled to a second end of the first lobe, and wherein the second end of the second connecting segment is coupled to a second end of the second lobe;
wherein the first lobe is curved between the first connecting segment and the second connecting segment, and wherein the second lobe is curved between the first connecting segment and the second connecting segment, and
wherein the first lobe and the second lobe are horseshoe shaped.

8. The implant of claim 5, wherein the first lobe is positioned to curve along a circumference of an exterior surface of the implant, and wherein the second lobe is positioned to curve along the circumference of the exterior surface of the implant,
wherein the first connecting segment and the second connecting segment are curved away from each other as the first and second connecting segments extend between the first lobe and the second lobe, and
wherein at least one trajectory extends through the at least one first intermediate portion at a top of the implant and the second intermediate portion at a bottom of the implant.

9. The implant of claim 8, wherein the second intermediate portion comprises:
a body;
a first lobe extending out from a first portion of the body; and
a second lobe extending out from a second portion of the body,
wherein the body has a circular shape.

10. The implant of claim 8, wherein the first lobe is spaced apart from the second lobe, and wherein the first lobe and second lobe are positioned between a first side and a second side,
wherein a first trajectory is formed between the first lobe of the at least one first intermediate portion and the second lobe of the second intermediate portion,
wherein a second trajectory is formed between the second lobe of the at least one first intermediate portion and the first lobe of the second intermediate portion, and
wherein the first trajectory crosses the second trajectory as the first and second trajectories extend between the top side and the bottom side of the implant.

11. The implant of claim 2, wherein the external frame comprises:
a first side;
a second side positioned opposite the first side;
a third side extending between a first end of the first side and a first end of the second side; and
a fourth side extending between a second end of the first side and a second end of the second side, and wherein the third side is opposite the fourth side.

12. The implant of claim 11, wherein the external frame further comprises:
a top side extending between and coupled to a first end of each of the first side, second side, third side, and fourth side; and
a bottom side extending between and coupled to a second end of each of the first side, second side, third side, and fourth side.

13. The implant of claim 11, wherein the first side includes at least one first window extending through the first side from an exterior surface of the first side into an interior of the implant, and
wherein the second side includes at least one second window extending through the second side from an exterior surface of the second side into the interior of the implant,
wherein the at least one first window is two identically sized and shaped first windows positioned as mirror images of each other on the first side, and
wherein the at least one second window is two identically sized and shaped second windows positioned as mirror images of each other on the second side,
wherein the third side includes at least one third window extending through the third side from an exterior surface of the third side into the interior of the implant, wherein the at least one third window comprises:
two medial third windows positioned on a medial side of the third side;
two lateral third windows positioned on a lateral side of the third side; and
one distal third window positioned near a bottom of the third side between at least one of the two medial third windows and at least one of the two lateral third windows; and
wherein the fourth side includes at least one fourth window extending through the third side from an exterior surface of the fourth side into the interior of the implant, wherein the at least one fourth window comprises:
two medial fourth windows positioned on a medial side of the fourth side;
two lateral fourth windows positioned on a lateral side of the fourth side; and
one distal fourth window positioned near a bottom of the fourth side between at least one of the two medial fourth windows and at least one of the two lateral fourth windows.

14. The implant of claim 13, wherein the third side further comprises:
a first protrusion extending away from an interior surface of the third side;
a first recessed region extending into the third side from the exterior surface and through a portion of the first protrusion toward the interior of the implant; and
a first through hole extending through the first protrusion from an interior surface of the first recessed region to the interior of the implant; and
wherein the fourth side further comprises:
a second protrusion extending away from an interior surface of the fourth side;
a second recessed region extending into the third side from the exterior surface and through a portion of the second protrusion toward the interior of the implant; and
a second through hole extending through the second protrusion from an interior surface of the second recessed region to the interior of the implant,
wherein the first recessed region has a larger width and height than the first through hole, and wherein the second recessed region has a larger width and height than the second through hole.

15. The implant of claim 11, wherein a first passageway is formed between a first end of the interior frame, the third side, a portion of the first side, and a portion of the second side, and wherein at least a portion of the lattice structure is formed within the first passageway; and
wherein a second passageway is formed between a second end of the interior frame, the fourth side, a portion of the first side, and a portion of the second side, and wherein at least a portion of the lattice structure is formed within the second passageway.

16. The implant of claim 2, wherein the external frame comprises:
a plurality of coupling portions positioned circumferentially spaced around the implant between the at least one first intermediate portion and the second intermediate portion; and
a plurality of arms positioned between and coupling each of the plurality of coupling portions together in a circular shape;
wherein the plurality of coupling portions comprises:
a first coupling portion on a first side of the implant;
a second coupling portion on a second side of the implant and opposite the first coupling portion;
a third coupling portion on a third side of the implant between the first coupling portion and the second coupling portion; and
a fourth coupling portion on a fourth side of the implant and opposite the third coupling portion;
wherein the plurality of arms comprises:
a first arm coupling the fourth coupling portion to the first coupling portion;
a second arm coupling the first coupling portion to the second coupling portion;
a third arm coupling the second coupling portion to the third coupling portion; and
a fourth arm coupling the third coupling portion to the fourth coupling portion, wherein the coupling portions and arms couple together to form a ring positioned near a midpoint between a top surface and a bottom surface of the implant.

17. The implant of claim 16, wherein the external frame further comprises:
a first crossing member coupled to the at least one first intermediate portion on a first end, the second intermediate portion on a second end, and the second arm near a midpoint of the first crossing member;
a second crossing member coupled to the at least one first intermediate portion on a first end, the second intermediate portion on a second end, and the third arm near a midpoint of the second crossing member; and
a third crossing member coupled to the at least one first intermediate portion on a first end and the second intermediate portion on a second end, wherein the third crossing member comprises:
a superior portion coupled to the at least one first intermediate portion on a first end and the third coupling portion on a second end; and
an inferior portion coupled to the third coupling portion on a first end and the second intermediate portion on the second end;
a plurality of openings extending from an exterior surface of the implant to an interior surface of the implant and positioned between the coupling portions, arms, and crossing members, wherein the lattice structure is formed within the plurality of openings.

18. A system, comprising:
an implant, comprising:
an interior frame;
an external frame surrounding the interior frame; and
a lattice structure extending between at least a portion of the interior frame and the external frame;
an insertion instrument coupled to the external frame of the implant; and
a guide removably coupled to the insertion instrument.

19. A surgical method, comprising:
exposing a patient's joint;
creating a cavity in the joint to receive an implant;
inserting trial implants into the cavity to select a desired implant size;
selecting the implant corresponding to the trial implant with the desire implant size;
coupling the implant to an insertion instrument;
inserting the implant coupled to the insertion instrument into the cavity;
manipulating the coupled implant and insertion instrument in at least one of rotation and translation to a desired position; and
fixing the implant to the joint.

20. The surgical method of claim 19, wherein creating a cavity in the joint to receive an implant comprises:
at least one of using at least one saw blade to cut the joint to create the cavity and using reaming instruments to create the cavity;
wherein fixing the implant to the joint comprises:
coupling a guide to the insertion instrument for inserting a fixation member;
wherein fixing the implant to the joint further comprises:
manipulating the coupled implant, insertion instrument, and guide in at least one of rotation and translation to the desired position;
wherein fixing the implant to the joint further comprises:
inserting a k-wire through the guide and across the joint through the implant;
removing the guide and insertion instrument from the implant;
drilling over the k-wire across the joint to form an opening;
inserting a cross screw into the opening, across the joint and through the implant;
attaching a plate to an exterior surface of the joint; and
inserting an intramedullary nail into the opening, across the joint and through the implant; and
wherein the implant is inserted into the patient's joint using at least one of an anterior, lateral, or posterior approach.
